(19) United States
(12) Patent Application Publication
(10) Pub. No.: US 2024/0182982 A1
(43) Pub. Date: Jun. 6, 2024

(54) FRAGMENTOMICS IN URINE AND PLASMA

(71) Applicant: Centre for Novostics, Shatin (HK)

(72) Inventors: Yuk-Ming Dennis Lo, Homantin (CN); Kwan Chee Chan, Jordan (CN); Peiyong Jiang, Pak Shek Kok (CN); Lizhen Ma, Pun Shan Chau (CN); Ze Zhou, Pak Shek Kok (CN)

(21) Appl. No.: 18/523,606

(22) Filed: Nov. 29, 2023

Related U.S. Application Data
(60) Provisional application No. 63/428,694, filed on Nov. 29, 2022.

(51) Int. Cl. C12Q 1/6886; C12Q 1/6858

(52) U.S. Cl. CPC C12Q 1/6886; C12Q 1/6858; C12Q 2600/112; C12Q 2600/154; C12Q 2600/156

(57) ABSTRACT

Fragmentomic features provide various properties of the sample (e.g., urine or plasma) and/or of a subject. Relative contributions or enrichment of clinically-relevant DNA (e.g., type(s) of transrenal and non-transrenal urinary cfDNA) are provided using fragmentomic features of urinary cell-free DNA. Such measurements could be used for reflecting the glomerular permeability and monitoring various diseases, e.g., kidney abnormality. The fragmentomic features can include a corrected urinary DNA concentration, size, end motifs of urinary DNA molecules, and cfDNA molecules from open chromatin regions (OCR) of one or more tissues. In addition, nuclease activities or other fragmentation processes of cfDNA are determined based on relative contributions concerning the different profiles of cfDNA cleavage, which are also used for determining contribution of cfDNA from tissue(s), level of pathology, and gestational age.

### CROSS-REFERENCES TO RELATED APPLICATION

This application is a nonprovisional of and claims the benefit of U.S. Provisional Patent Application No. 63/428,694, entitled “FRAGMENTOMICS IN URINE AND PLASMA,” filed on Nov. 29, 2022, which is herein incorporated by reference in its entirety for all purposes.

### BACKGROUND

Urinary cell-free DNA and plasma cell-free DNA molecules include DNA molecules released from different normal tissues/organs and malignant cells but exhibit distinct fragmentation patterns. For example, the urinary cell-free DNA molecules have generally shorter size profiles enriched with sharper 10-bp periodic peaks apart compared with plasma cell-free DNA (Tsui et al., PLOS ONE 2012; 7:e48319). Deoxyribonuclease 1 like 3 (DNASE1L3) was demonstrated to be the major contributor to plasma cell-free DNA fragmentation using the mice models with gene deletion of the DNases (Han et al. Am J Hum Genet. 2020; 106:202-214). In contrast to plasma samples, deoxyribonuclease 1 (DNASE1) was responsible for shaping the urinary cell-free DNA fragmentomic properties (Chen et al. PLOS Genet. 2022; 18:e1010262).

Urinary cell-free DNA can also include different types of DNA molecules having their respective characteristics. For example, there are “transrenal” urinary cell-free DNA molecules that are released from the non-urinary system (e.g., blood cells, liver, lung, colon, heart, brain, spleen, stomach, placenta tissues), which pass through glomerulus of the kidney to the urinary system. In addition to the transrenal cell-free DNA, there are also “non-transrenal” urinary cell-free DNA molecules that originate and are directly released from the urinary system, such as kidney tubules, bladder, urethra, etc. However, there is a lack of methods for identifying the characteristics or reflecting the extent of transrenal and non-transrenal cell-free DNA from a given urine sample.

In addition, many studies demonstrated that the use of plasma end motifs could inform the presence of various diseases ranging from autoimmune diseases to multiple cancer types (Chan et al. Am J Hum Genet. 2020; 107:882-894; Jiang et al. Cancer Discov. 2020; 10:664-73). Therefore, it can be clinically meaningful to holistically determine the usage levels of nucleases, such as DNASE1L3, DNASE1, DNA fragmentation factor subunit beta (DFFB), etc. We reasoned that the use of end-motif profiles would allow for deducing the extent of nucleases involved in the generation of cell-free DNA molecules (i.e., the nuclease usage level) and monitoring the nuclease activities across different pathophysiological statuses. However, there is a paucity of tools permitting a comprehensive assessment of various DNA nucleases in a single analysis.

### SUMMARY

Methods, apparatuses, and systems are provided for fragmentomic features of a sample for determining various properties of the sample and/or of a subject. Various methods may be used for urine samples and/or plasma samples.

As examples for urine samples, fractional concentration or enrichment of clinically-relevant DNA, e.g., type(s) of transrenal and non-transrenal urinary cfDNA, can be provided using fragmentomic features of urinary cell-free DNA. Measurements of urinary DNA fragmentomics can also be used for reflecting the glomerular permeability and monitoring various diseases, e.g., kidney abnormality. The fragmentomic features can include a corrected urinary DNA concentration, size, end motifs of urinary DNA molecules, and cfDNA molecules from open chromatin regions (OCR) of one or more tissues.

In other embodiments (e.g., for urine, plasma, or other cell-free samples), nuclease activities or other fragmentation processes of cfDNA can be determined based on relative contributions of different profiles of cfDNA cleavage, which can also be used for determining fractional concentration of cfDNA from tissue(s), level of pathology, and gestational age.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

Reference to the remaining portions of the specification, including the drawings and claims, will realize other features and advantages of the present disclosure. Further features and advantages of the present disclosure, as well as the structure and operation of various embodiments of the present disclosure, are described in detail below with respect to the accompanying drawings. In the drawings, like reference numbers can indicate identical or functionally similar elements.

### BRIEF DESCRIPTION OF THE DRAWINGS

FIG. 1 illustrates an example overview that identify characteristics of transrenal and non-transrenal DNA in urine samples, according to some embodiments.

FIG. 2 shows a schematic diagram illustrating determination of transrenal urinary cell-free DNA contribution using fragmentomic features.

FIG. 3 illustrates a process in which sequencing data are obtained from urine samples, according to some embodiments.

FIG. 4 shows a set of graphs that show comparisons of urinary cell-free DNA concentrations comparisons between before and after in-vitro incubation among three urine collection groups.

FIG. 5 shows a set of graphs identifying comparisons of urinary cell-free DNA size profiles before and after in-vitro incubation among control, EDTA, and stabilizer groups.

FIG. 6 shows a graph that identifies size differences between fetal DNA and maternal DNA in urine samples, according to some embodiments.

FIG. 7 illustrates an example schematic diagram that shows a biological process of converting plasma DNA into transrenal DNA, according to some embodiments.

FIG. 8 shows a graph that identifies a relationship between glomerular basement membrane permeability and sizes of transrenal DNA, according to some embodiments.

FIG. 9 shows a graph that identify different end-motifs identified from fetal-specific and shared cell-free DNA in maternal urine.

FIG. 10 shows a set of graphs that identify a relationship between fractional concentration of fetal DNA with CC-end fragments in urinary cell-free DNA and having different sizes, according to some embodiments.

FIG. 11 illustrates an example diagram that identifies various characteristics of DNA molecules from open chromatin regions, according to some embodiments.

FIG. 12 illustrates an example diagram for determining amounts of urinary cell-free DNA corresponding to open chromatin regions, according to some embodiments.

FIG. 13 shows a set of boxplots of O/E ratios of fetal-specific and shared cell-free DNA molecules in urine samples and plasma samples.

FIG. 14 illustrates a set of graphs that identify enrichment of DNA molecules in open chromatin regions in fetal-specific DNA from urine samples, according to some embodiments.

FIG. 15 illustrates a set of graphs that identify non-enrichment of DNA molecules in open chromatin regions in fetal-specific DNA from plasma samples, according to some embodiments.

FIG. 16 shows a graph that identifies a correlation between fetal DNA fraction in maternal urine and the O/E ratio of all urinary cell-free DNA fragments.

FIG. 17 shows a graph illustrating a correlation between fetal DNA fraction in maternal urine and the O/E ratio of urinary cfDNA fragments from placenta-specific DHSs.

FIG. 18 shows a graph that identifies the normalized end density of all urinary cell-free DNA in OCR of maternal urine and plasma samples.

FIG. 19 shows a set of graphs that identify a comparison between end density of urinary cell-free DNA and plasma cell-free DNA for determining fractional concentration of fetal DNA, according to some embodiments.

FIG. 20 shows a set of graphs that identify correlations between fetal DNA fraction and the normalized end density of urinary cell-free DNA having different sizes, according to some embodiments.

FIG. 21 shows a flowchart for estimating a fractional concentration of clinically-relevant DNA molecules in a urine sample of a subject, according to some embodiments.

FIG. 22 shows a set of graphs that identify correlations between fetal DNA fraction and proportion of urinary cell-free DNA fragments carrying CC-ends.

FIG. 23 illustrates a technique using probes for enriching a set of one or more end motifs.

FIG. 24 illustrates another technique using probes and beads for enriching a set of one or more end motifs.

FIG. 25 shows a flowchart for enriching a urine sample for clinically-relevant DNA based on end-motif characteristics of urinary cell-free DNA, according to some embodiments.

FIG. 26 shows a set of graphs that identify enrichment of fetal DNA using urinary cell-free DNA having various fragmentomic properties, according to some embodiments.

FIG. 27 shows a bar graph that identifies enrichment of transrenal urinary cell-free DNA using selective analysis of fragments with different fragmentomic features.

FIG. 28 shows a flowchart for enriching a urine sample for clinically-relevant DNA based on end motifs, open chromatin regions, and size of urinary cell-free DNA, according to some embodiments.

FIG. 29 shows a set of graphs that identify O/E ratio analysis in patients with RCC.

FIG. 30 shows fragmentomic analysis of transrenal DNA in the patients with proteinuria using blood-specific DHSs.

FIG. 31 shows fragmentomic analysis of transrenal DNA in the pregnant women with preeclampsia using DHSs.

FIG. 32 shows a flowchart for determining a classification of kidney abnormality based on urinary cell-free DNA from open chromatin regions, according to some embodiments.

FIG. 33 is a fragmentomic analysis of transrenal DNA in the patients with proteinuria and separately with preeclampsia.

FIG. 34 shows a flowchart for determining a classification of kidney abnormality based on sizes of urinary cell-free DNA, according to some embodiments.

FIG. 35 shows urinary cfDNA concentration analysis of transrenal DNA in the patients with proteinuria and preeclampsia, separately.

FIG. 36 shows a flowchart for determining a classification of kidney abnormality based on urinary cell-free DNA concentration, according to some embodiments.

FIG. 37 shows an ROC analysis in differentiating patients with proteinuria and preeclampsia from healthy controls using fragmentomic features of transrenal DNA.

FIG. 38 shows a plot graph that identifies a ranking of frequencies of certain end motifs present in urinary cell-free DNA molecules, according to some embodiments.

FIG. 39 shows a set of graphs that identify observed end-motif profiles of murine plasma and urinary cell-free DNA molecules.

FIG. 40 shows a schematic workflow of an example nuclease-usage level analysis for cell-free DNA molecules.

FIG. 41 shows a diagram that identifies proportional contribution of each F-profile (i.e., nuclease usage level) deduced from murine cell-free DNA samples with different knockout genotypes using NMF analysis.

FIG. 42 shows a set of plots for six F-profiles (A-F) deduced from mouse plasma and urinary cell-free DNA using NMF analysis.

FIG. 43 shows a boxplot that identifies proportional contribution of F-profile I across different types of samples, according to some embodiments.

FIG. 44 shows relative frequencies of cell-free DNA molecules across 256 end motifs for F-profile I, according to some embodiments.

FIG. 45 shows a boxplot that identifies proportional contribution of F-profile II across different types of samples, according to some embodiments.

FIG. 46 shows relative frequencies of cell-free DNA molecules across 256 end motifs for F-profile II, according to some embodiments.

FIG. 47 shows a boxplot that identifies proportional contribution of F-profile III across different types of samples, according to some embodiments.

FIG. 48 shows relative frequencies of cell-free DNA molecules across 256 end motifs for F-profile III, according to some embodiments.

FIG. 49 shows relative frequencies of cell-free DNA molecules across 256 end motifs for F-profiles IV-VI, according to some embodiments.

FIG. 50 shows a schematic diagram of comparing an end-motif profile of a human subject to reference F-profiles determined based on murine samples, according to some embodiments.

FIG. 51 shows proportional contributions of F-profiles across plasma and urine samples of human control subjects, according to some embodiments.

FIG. 52 shows proportional contributions of F-profiles across plasma samples of normal and DNASE1L3-deficient human subjects, according to some embodiments.

FIG. 53 shows proportional contributions of F-profiles across urine samples of pregnant human subjects, according to some embodiments.

FIG. 54 shows a flowchart for determining a classification of nuclease activity based on F-profiles of cell-free DNA molecules, according to some embodiments.

FIG. 55 shows a set of graphs that identify the nuclease usage levels in urinary cell-free DNA of pregnant women.

FIG. 56 shows a flowchart for determining fractional concentration of fetal DNA based on F-profiles of cell-free DNA molecules, according to some embodiments.

FIG. 57 shows a set of graphs that identify the nuclease usage levels in plasma cell-free DNA of pregnant women.

FIG. 58 shows a set of graphs that identify F-profile analysis and oxidative stress level in pregnant women.

FIG. 59 shows a flowchart for estimating gestational age based on F-profiles of cell-free DNA molecules, according to some embodiments.

FIG. 60 shows a boxplot of F-profile I (DNASE1L3) levels for healthy subjects, patients with DNASE1L3 deficiency, and parents of the patients.

FIG. 61 shows a set of graphs that identify nuclease usage levels in plasma cell-free DNA of subjects with and without systemic lupus erythematosus (SLE).

FIG. 62 shows proportional contributions of F-profiles across normal, HBV, and HCC plasma samples, according to some embodiments.

FIG. 63 shows a set of graphs that show nuclease usage levels in plasma cell-free DNA of subjects with and without HCC.

FIG. 64 shows a bar graph that identifies oxidative stress levels in the blood samples from controls and HCC patients.

FIG. 65 shows a set of graphs that provide F-profile analysis and oxidative stress level in CRC patients.

FIG. 66 shows a boxplot that identifies F-profile VI contributions in NPC patients before and during chemoradiotherapy treatment with cisplatin.

FIG. 67 shows a flowchart for determining a classification of a level of pathology based on F-profiles of cell-free DNA, according to some embodiments.

FIG. 68 illustrates a measurement system according to an embodiment of the present disclosure.

FIG. 69 shows a block diagram of an example computer system usable with system and methods according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any sample that is taken from a subject (e.g., a human (or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g. of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g. thyroid, breast), intraocular fluids (e.g. the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 1,600 g×10 minutes, obtaining the fluid part, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed.

A “sequence read” refers to a string of nucleotides sequenced from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed. An amount of sequence reads can be used as a proxy for the number of DNA fragments. To determine the number of DNA fragments from the amount of sequence reads, a calculation may be performed to account for paired-end sequencing and/or bias of sequencing techniques.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment, and thus be part of or include an ending sequence. An “end motif” can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif.

The term “mapping” refers to a process which relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location.

A “reference genome” may be an entire genome sequence of a reference organism, a portion of a reference genome, a consensus sequence of many reference organisms, a compilation sequence based on different components of different organisms, or any other appropriate reference sequence. A reference may also include information regarding variations of the reference known to be found in a population of organisms.

A “rate” of DNA molecules ending on a position relates to how frequently a DNA molecule ends on the position. Such a rate can be referred to as an “end density.” The rate may be based on a number of DNA molecules that end on the position normalized against a number of DNA molecules analyzed. The normalization can also be based on the average, median, or total number of ends in the surrounding region. The surrounding region used for normalization may include, but is not limited to, 500, 1000, 3000, 5000, etc. bp upstream and/or downstream of the position.

A “relativefrequency” (also referred to just as “frequency”) may refer to a proportion (e.g., a percentage, fraction, or concentration). In particular, a relative frequency of a particular end motif (e.g., CCGA or just a single base) can provide a proportion of cell-free DNA fragments in a sample that are associated with the end motif CCGA, e.g., by having an ending sequence of CCGA.

The terms “control”, “control sample”, “background sample,” “reference”, “reference sample”, “normal”, and “normal sample” may be interchangeably used to generally describe a sample that does not have a particular condition (a kidney abnormality) or is otherwise healthy. In an example, the reference sample is a sample taken from a subject without a condition. A reference sample may be obtained from the subject, or from a database.

“Clinically-relevant DNA” can refer to DNA of a particular tissue source that is to be measured, e.g., to determine a fractional concentration of such DNA or to classify a phenotype of a sample (e.g., plasma). Herein, clinically-relevant DNA can refer to transrenal DNA that exists before passing through a kidney, as opposed to non-transrenal DNA (such as from the kidney or bladder). Examples of clinically-relevant DNA are fetal DNA (e.g., from maternal plasma) or tumor DNA (e.g., from a patient's plasma). Another example includes the measurement of the amount of graft-associated DNA in urine of a transplant patient. A further example includes the measurement of the fractional concentration of a liver DNA fragments (or other nonhematopoietic tissue or hematopoietic tissue, e.g., blood cells) in a sample or fractional concentration of brain DNA fragments in cerebrospinal fluid.

A “calibration sample” can correspond to a biological sample whose desired measured value (e.g., nuclease activity, fractional concentration of clinically-relevant nucleic acid, classification of a genetic disorder, or other desired property) is known or determined via a calibration method, e.g., ELISA for measuring nuclease quantity or assays quantifying the rate of DNA digestion by nucleases for measuring nuclease activity (an example method can involve fluorometric or spectrophotometric measurement of DNA quantity before and after, or in real-time, the addition of a nuclease-containing sample; another example is using radial enzyme diffusion methods). The fractional concentration of clinically-relevant DNA (e.g., tissue-specific DNA fraction) can be known, e.g., as determined via a calibration method, such as using a tissue-specific allele. For example, for a tumor, a fetus, or transplantation, an allele present in the tissue's (e.g., donor's genome) but absent in the healthy/maternal/recipient's genome can be used as a marker for the tissue corresponding to the clinically-relevant DNA. As another example, a tissue-specific methylation pattern can be used. A calibration sample can have separate measured values (e.g., an amount of fragments with a particular end motif or with a particular size) can be determined to which the desired measure value can be correlated.

A “calibration data point” includes a “calibration value” (e.g., an amount of fragments with a particular end motif or with a particular size) and a measured or known value that is desired to be determined for other test samples. The calibration value can be determined from various types of data measured from DNA molecules of the sample, (e.g., an amount of fragments with an end motif or with a particular size). The calibration value corresponds to a parameter that correlates to the desired property, e.g., classification of a genetic disorder, nuclease activity, or efficacy of anticoagulant dosage. For example, a calibration value can be determined from measured values as determined for a calibration sample, for which the desired property is known. The calibration data points may be defined in a variety of ways, e.g., as discrete points or as a calibration function (also called a calibration curve or calibration surface). The calibration function could be derived from additional mathematical transformation of the calibration data points.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample.

A “site” (also called a “genomic site”) corresponds to a single site, which may be a single base position or a group of correlated base positions, e.g., a CpG site, TSS site, DNASE hypersensitivity site, or larger group of correlated base positions. A “locus” may correspond to a region that includes multiple sites. A locus can include just one site, which would make the locus equivalent to a site in that context.

The term “open chromatin regions (OCR)” refers to one or more sites that correspond to nucleosome-deleted regions (i.e. a lack of histone-bound DNA). In some instances, an OCR includes one or more DNase1 hypersensitive sites (DHS) defined using DNase-seq (Meuleman et al. Nature. 2020; 584:244-251). As examples, OCR can be defined based on sites identified using DNase-seq, sites identified using Assay for Transposase-Accessible Chromatin using sequencing (ATAC-seq), transcriptional start sites (TSS), CCCTC-binding factor (CTCF) sites, enhancer sites, histone modifications marked regions (e.g. H3K27ac, H3K4me3, etc.), as well as other nuclease hypersensitive sites. In some instances, OCR could be a region with a relative decrease of nucleosome occupancy. In some instances, OCR can be tissue-specific. In various embodiments, at least 100, 500, 1,000, 5,000, or 10,000 OCRS can be used in embodiments described herein.

The term “kidney abnormality” refers to a disorder that affects the kidney and potentially other organs. As examples, the kidney abnormality can include renal cell carcinoma (RCC), nephrotic syndrome, glomerulonephritis, Fabry disease, cystinosis, IgA nephropathy, IgM nephropathy, lupus nephritis, atypical hemolytic uremic syndrome (aHUS), polycystic kidney disease (PKD), Alport's syndrome, interstitial nephritis, proteinuria, chronic kidney disease (CKD), acute kidney injury, preeclampsia, etc.

A “end-motif profile” may refer to the relationship of ending sequences (e.g., 1-30 bases) of cell-free DNA fragments (also just referred to as DNA fragments) in a sample. Various relationships can be provided, e.g., an amount of cell-free DNA fragments with a particular ending sequence (end motif), a relative frequency of cell-free DNA fragments with a particular ending sequence compared to one or more other ending sequences. In some instances, the end-motif profiles are determined using other types of parameters, such as size. For example, the end-motif profile can be provided in various ways that illustrate an amount of cell-free DNA fragments having one or more particular ending sequences for a given size (single length or size range). A “reference end-motif profile” or an “F-profile” refers to an end-motif profile that can be generated by applying a factorization algorithm (e.g., non-negative matrix factorization) to relative frequencies of DNA molecules of a given biological sample across a plurality of end motifs (e.g., 256 end motifs).

The term “relative abundance” may generally refer to a ratio of a first amount of nucleic acid fragments having a particular characteristic (e.g., a specified length, ending at one or more specified coordinates/ending positions, or aligning (mapping) to a particular region of the genome) to a second amount nucleic acid fragments having a particular characteristic (e.g., a specified length, ending at one or more specified coordinates/ending positions, or aligning (mapping) to a particular region of the genome). In one example, relative abundance may refer to a ratio of the number of DNA fragments ending at a first set of genomic positions (e.g., open chromatin regions) to the number (e.g., a mean or a median) of DNA fragments ending at a second set of genomic positions, which may be all genomic positions. Such a relative abundance may be referred to as an end density. In some aspects, “relative abundance” is a type of separation value that relates an amount (one value) of cell-free DNA molecules ending within one window of genomic position to an amount (other value) of cell-free DNA molecules ending within another window of genomic positions. The two windows may overlap but would be of different sizes. In other implementations, the two windows would not overlap. Further, the windows may be of a width of one nucleotide, and therefore be equivalent to one genomic position. An end density is a type of relative abundance. In some instances, an observed-to-expected (O/E) ratio is another type of relative abundance.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1).

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis.

The terms “cutoff” and “threshold” refer to predetermined numbers used in an operation. For example, a size cutoff (or size threshold) can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

A “level of pathology” (or level of a disorder) can refer to the amount, degree, or severity of pathology associated with an organism. An example is a cellular disorder in expressing a nuclease. Another example of pathology is a rejection of a transplanted organ. Other example pathologies can include autoimmune attack (e.g., lupus nephritis damaging the kidney or multiple sclerosis), inflammatory diseases (e.g., hepatitis), fibrotic processes (e.g. cirrhosis), fatty infiltration (e.g. fatty liver diseases), degenerative processes (e.g. Alzheimer's disease) and ischemic tissue damage (e.g., myocardial infarction or stroke). A heathy state of a subject can be considered a classification of no pathology. The pathology can be cancer.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g. recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g. symptoms or other positive tests), has cancer.

The name of a gene is typically written in italics. A human gene is typically also written in all capital letters. A mouse gene may not be capitalized after the first letter. The protein is conventionally written in all capital letters and without italics. As examples, a mouse may have the Dnase1l3 gene and the DNASE1L3 protein, while a human may have the DNASE1L3 gene and the DNASE1L3 protein.

A “machine learning model” (ML model) can refer to a software module configured to be run on one or more processors to provide a classification or numerical value of a property of one or more samples. An ML model can be generated using sample data (e.g., training data) to make predictions on test data. One example is an unsupervised learning model. Another example type of model is supervised learning that can be used with embodiments of the present disclosure. Example supervised learning models may include different approaches and algorithms including analytical learning, statistical models, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to +10%. The term “about” can refer to +5%.

### DETAILED DESCRIPTION

Various fragmentomic features of cell-free samples (e.g., urine and plasma) are used for determining various properties of the sample and/or of a subject.

As examples for urine samples, some embodiments can detect contributions of transrenal and non-transrenal urinary cell-free DNA using fragmentomic features of urinary cell-free DNA. Such measurement could be used for reflecting the glomerular permeability and monitoring various diseases, such as but not limited to kidney abnormalities, e.g., kidney cancer and kidney diseases, as well as proteinuria and preeclampsia, which can be classified as types of a kidney abnormality.

In addition, such measurement can also be used to determine fractional concentration of clinically relevant cell-free DNA molecules, as well as enriching a urine sample for clinically-relevant DNA, including all types of transrenal DNA (e.g., liver-, lung-, colon-, heart-, and blood-derived, e.g., white blood cells), fetal DNA, tumor DNA, or DNA from a particular tissue other than from urinary tract, such as kidney, ureters, and bladder). The relative contribution of transrenal cell-free DNA can be determined by determining a relative abundance of cell-free DNA molecules that are from open chromatin regions of one or more tissues, such as all or a representative sampling of OCRs (e.g., at least 100, 500, 1,000, 5,000, or 10,000 OCRs) or one or more tissues that contribute to any transrenal DNA. In other instances, the relative contribution or enrichment of transrenal cell-free DNA molecules can determined based on cell-free DNA molecules from a urine sample having particular sizes and/or end motifs, as well as a corrected urine concentration.

For instance for end motifs, some embodiments can use the existence of a C at the end of a cfDNA molecule to enrich a sample for clinically-relevant DNA. Thus, the transrenal cell-free DNA contribution in urine could be determined using fragmentomic features such as end signatures and abundance of cfDNA from transrenal-specific open chromatin regions in urinary cell-free DNA according to embodiments of the present disclosure.

Furthermore, different types of cell-free DNA cleavage can be analyzed simultaneously (i.e., together) using end motifs. The different types can be distinct by representing different dimensions in a cleavage space representing all the nuclease activity that can occur in a subject. In this disclosure, different types of cell-free DNA cleavage are linked to different fragmentation processes, including enzymatic and non-enzymatic breakages, based on nuclease knockout mice and/or human subjects with various drug treatments.

In contrast to the techniques that focused on one specific nuclease activity each time using one end motif or several top-ranked end motifs (Serpas et al. Proc. Natl. Acad. Sci. USA. 2019; 116:641-649; Chan et al. Am. J. Hum. Genet. 2020; 107:882-894; Chen et al. PLOS Genet. 2022; 18:e1010262), embodiments of the present disclosure can simultaneously assess a number of nuclease activities or other fragmentation processes that might be involved (e.g. induced by chemoradiotherapy), on the basis of deduced relative contributions concerning the different types of cell-free DNA cleavage. The contributions of each type of cell-free DNA cleavage can be determined by generating a set of F-profiles that represent the relative frequencies of end motifs for a given biological sample. In some instances, the set of F-profiles can be generated by applying factorization (e.g., non-negative matrix factorization) to the relative frequencies. The analysis of perturbed contributions could allow for the detection and monitoring of various diseases but not limited to cancers and immune diseases.

Accordingly, as described herein, end motifs (e.g., sample end-motif profiles and reference profiles, referred to as reference F-profiles) can be used in various ways to determine a property of a sample and/or a classification of a subject, such as determining a fractional concentration of clinically-relevant DNA, a gestational age of a fetus, or a level of pathology of a subject.

### I. OVERVIEW

The glomerular basement membrane (GBM) allows plasma cell-free DNA to pass through the kidney and become transrenal cell-free DNA. Generally, smaller DNA molecules have a greater GBM permeability over larger DNA molecules. For example, the permeability of the GBM decreased as the size of the molecules that come from plasma to urine increased (Lawrence et al. Proc Natl Acad Sci USA. 2017; 114:2958-2963). Further, nucleosome-depleted DNA molecules (e.g., DNA molecules from nucleosome-depleted regions) may have a smaller molecular size than nucleosomal DNA with the same DNA length due to the attachment of histones to the DNA. In some embodiments, the enrichment of nucleosome-depleted DNA in transrenal cell-free DNA is used for determining a level of glomerular permeability.

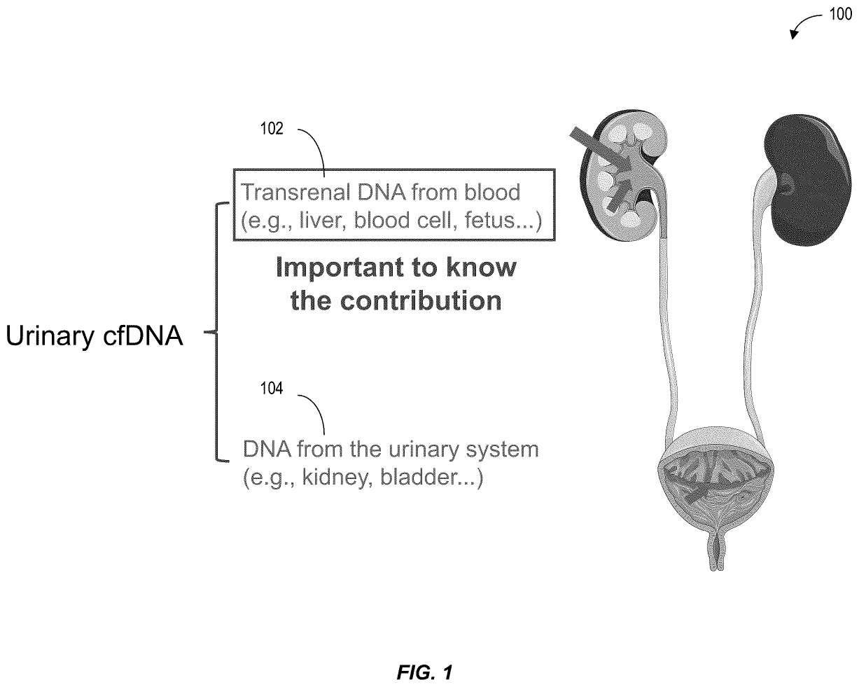

FIG. 1 illustrates an example overview 100 that identify characteristics of transrenal and non-transrenal DNA in urine samples, according to some embodiments. As shown in FIG. 1, the urinary cell-free DNA includes transrenal DNA 102 that are from the plasma and passes through the kidney into the urinary system. For example, transrenal DNA may originate from the liver, blood cell, a tumor, or fetal DNA. The urinary cell-free DNA can also include non-transrenal DNA 104 that are from the urinary system. For example, the non-transrenal cell-free DNA can originate from the urinary tract (e.g., kidney and bladder). The two types of urinary cell-free DNA may be associated with different characteristics. By identifying the differences between transrenal cell-free DNA (cfDNA) and the non-transrenal cell-free DNA, contributions of transrenal cell-free DNA or non-transrenal cell-free DNA can be determined from a given urine sample. Transrenal cfDNA in general or a certain type of transrenal DNA may be considered clinically-relevant DNA. Thus, a contribution of transrenal cfDNA in total or a particular type (e.g., fetal, tumor, or from a particular organ or type of blood cells) can be determined from such differences.

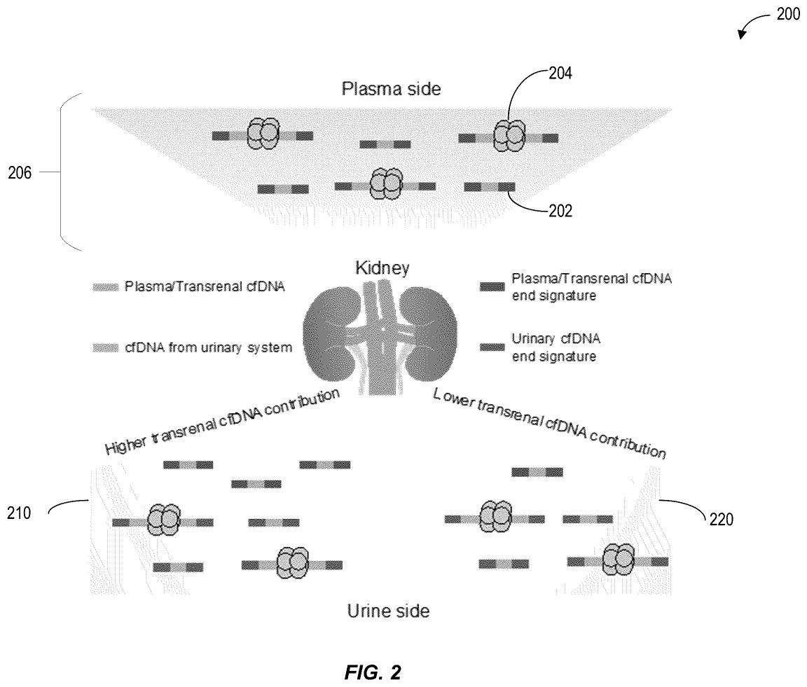

FIG. 2 shows a schematic diagram 200 illustrating determination of transrenal urinary cell-free DNA contribution using fragmentomic features. Both nucleosome-depleted cell-free DNA 202 (i.e., without associated proteins, such as histones) and nucleosomal cell-free DNA molecules 204 are present in the plasma 206. When the plasma DNA molecules 206 pass through the GBM in the kidney, nucleosome-depleted cell-free DNA molecules 202 have higher permeability compared with nucleosomal DNA molecules 204, which have a larger molecular size. Meanwhile, when entering the urinary cell-free DNA pool, the transrenal urinary cell-free DNA could still carry the end signatures formed in plasma, for example, mediated by DNASE1L3. Thus, the transrenal cell-free DNA contribution in urine could be determined using fragmentomic features such as end signatures and abundance of cfDNA from transrenal-specific open chromatin regions in urinary cell-free DNA according to embodiments of the present disclosure.

FIG. 2 shows two illustrative examples for on the urine side. A first urine sample 210 has higher transrenal cfDNA contribution, as shown by having 3 of the 7 DNA fragments being transrenal urinary cfDNA. A second urine sample 220 has lower transrenal cfDNA contribution, as shown by having only 1 of the 5 DNA fragments being transrenal urinary cfDNA. Various embodiments can differentiate between such samples (and even differentiate among different types of transrenal DNA), e.g., as part of estimating fractional concentration of clinically-relevant DNA, determining a classification of a pathology, such as a kidney abnormality, or detecting preeclampsia or proteinuria, which can be classified as types of a kidney abnormality.

However, challenges exist. The fragmentomics of transrenal cell-free DNA is generally poorly understood. In addition, urinary cell-free DNA and plasma cell-free DNA fragmentation processes may involve different nucleases (Han et al. Am J Hum Genet. 2020; 106:202-214; Chen et al. PLOS Genet. 2022; 18:e1010262). For example, DNASE1L3 is the predominant nuclease for generating C-end fragments in plasma (Serpas et al. Proc Natl Acad Sci USA. 2019; 116:641-649), while DNASE1 is responsible for generating T-end fragments in urine (Chen et al. PLOS Genet. 2022; 18:e1010262).

Based on such differences, we hypothesized that the transrenal urinary cell-free DNA molecules would carry the end motif signatures of those cell-free DNA present in plasma. In effect, the analysis of end motifs in urinary cell-free DNA can be used for inferring the contribution of transrenal cell-free DNA. For example, a higher amount of urinary cell-free DNA carrying C-ends that is one signature of plasma DNA can be suggestive of a higher contribution of transrenal cell-free DNA.

In view of the above, there is a need for understanding the fragmentomic differences (e.g., size, end motifs) between transrenal and non-transrenal cell-free DNA. By identifying such differences, transrenal cell-free DNA contribution can be accurately estimated without any genetic or epigenetic information (e.g., SNPs from tumor tissue). The transrenal cell-free DNA contribution can then be applied to a disease model. For example, a subject with kidney functions may either have higher or lower transrenal contribution than the normal subjects.

### II. EXAMPLE URINE SAMPLE PREPARATION

Various techniques can be used to prepare a urine sample for analyzing the cfDNA. The techniques described below are only examples as will be appreciated by a person skilled in the art.

### A. Sample Collection

To determine differences between transrenal and non-transrenal DNA, cell-free DNA molecules obtained from plasma and urine samples can be analyzed. For example, 192 human plasma and 18 urinary cell-free DNA samples were sequenced using paired-end sequencing. In particular, the plasma and urinary cell-free DNA samples included: (i) urinary cell-free DNA samples from pregnant women (n=20), urinary cell-free DNA samples from preeclampsia (n=5), and plasma cell-free DNA samples from pregnant women (n=11) (median number of paired-end reads: 129.5 million; range: 30.1-234.9 million); (ii) urinary cell-free DNA samples from renal cell carcinoma (RCC) (n=16), proteinuria (n=24) and controls (n=34) (median number of paired-end reads: 25.03 million; range: 13.34-75.02 million); (iii) plasma cell-free DNA samples from 8 healthy individuals, 10 patients with DNASE1L3 disease-associated variants, 3 parents of the patients with mutant DNASE1L3 gene (median number of paired-end reads: 108 million; range: 40-162 million); (iv) plasma cell-free DNA samples from 24 SLE patients and 11 healthy individuals (median paired-end reads: 120 million; range: 18-208 million); (v) plasma cell-free DNA samples from 38 healthy individuals, 17 patients with chronic hepatitis B virus (HBV) but without hepatocellular carcinoma (HCC) (i.e., HBV carriers), and 34 patients with HCC (median paired-end reads: 38 million; range: 18-65 million); (vi) plasma cell-free DNA from 30 pregnant women across first trimester (12-14 weeks; n=10), second trimester (20-24 weeks; n=10), and third trimester (38-40 weeks; n=10) (median number of paired-end reads: 103 million; range: 52-186 million); (vii) plasma cell-free DNA samples from 15 healthy control subjects, 25 colorectal cancer (CRC) patients without liver metastasis, and 24 CRC patients with liver metastasis (median number of paired-end reads: 40 million; range: 16-89 million); and (viii) plasma cell-free DNA samples from patients with nasopharyngeal carcinoma subjected to the chemoradiotherapy with Cisplatin (n=6) and paired patients before the treatment (n=6) (median number of paired-end reads: 5 million; range: 3-9 million).

### B. Using Stabilizers on Urine Samples

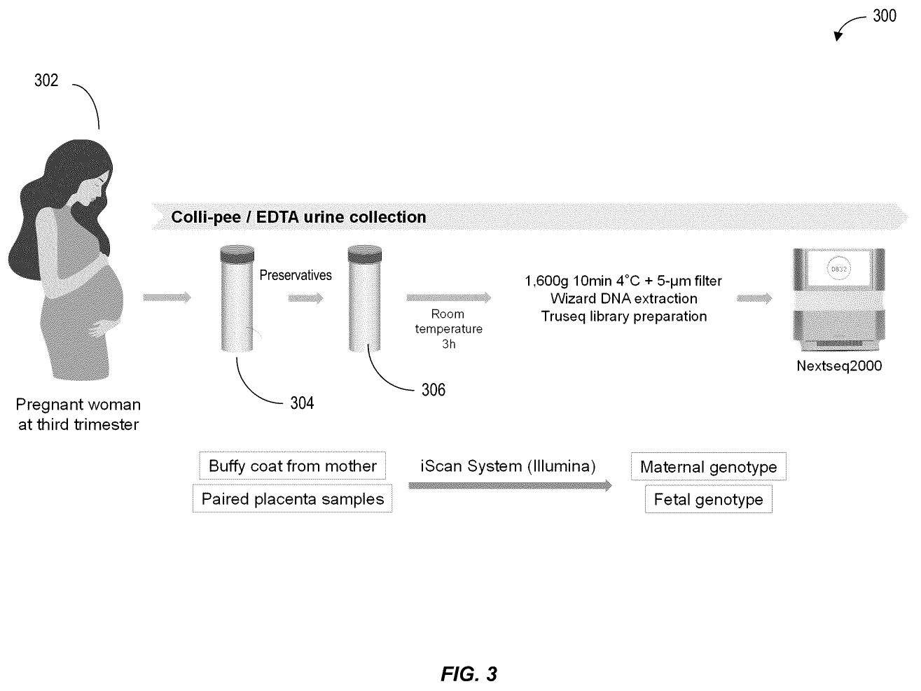

FIG. 3 illustrates a process 300 in which sequencing data are obtained from urine samples, according to some embodiments. At block 302, urine samples from pregnant women were used (e.g., third trimester sample). Such samples were used to determine whether transrenal contribution can be correlated with fetal DNA contribution.

Sequencing urine samples can be challenging, because DNASE1 activity is overwhelmingly high in the urine. If the DNASE1 activity is not completely inhibited after the urine collection, the in-vitro continuous fragmentation caused by DNASE1 could confound the fragmentation patterns originally present in urine, which might reduce the fragmentomic signals of urinary DNA fragments related to a particular disease.

To address the above challenges, various collection and preservation methods can be used for better preserving the original characteristics of urinary cell-free DNA. For example, as shown in block 304, preservatives can be added to get a preserved sample at block 306. Different urine collection methods can be used, including adding Ethylenediaminetetraacetic acid (EDTA) and adding stabilizers. EDTA can inhibit the cleavage activity of the DNASE1 family by chelating magnesium and calcium, which are the essential ions required for DNASE1 digestion. The stabilizers can potentially stabilize the urinary DNA from degradation. The stabilizers could be but not limited to preservatives provided by Collipee company, diazolidinyl urea (DU), dimethylolurea, 2-bromo-2-nitropropane-1,3-diol, 5-hydroxymethoxymethyl-1-aza-3,7-dioxabicyclo (3.3.0) octane and 5-hydroxymethyl-1-aza-3,7-dioxabicyclo (3.3.0) octane and 5-hydroxypoly[methyleneoxy]methyl-1-aza-3,7-dioxabicyclo (3.3.0) octane, bicyclic oxazolidines (e.g. Nuosept95), DMDM hydantoin, imidazolidinyl urea (IDU), sodium hydroxymethylglycinate, hexamethylenetetramine chloroallyl chloride (Quaternium-15), biocides (such as Bioban, Preventol and Grotan), a water-soluble zinc salt, EDTA, other metal ion chelators such as N, N′-bis-(dithiocarboxy) piperazine (BDP), diethyldithiocarbamate (DDTC), iminodisuccinic acid (IDS), polyaspartic acid, S,S-Ethylenediamine-N,N′-disuccinic acid (EDDS), methylglycinediacetic acid (MGDA), etc.

Urinary cell-free DNA can be preserved better in a device containing stabilizers compared with those without stabilizers. As an illustrative example, urinary cell-free DNA samples from 2 control subjects were collected with different collection methods (no addition of agents, EDTA, and stabilizer groups) under room temperature in-vitro incubation for different time durations (e.g. 0-hour and 4-hour incubation). The cell-free DNA concentrations and size profiles were compared among three urine collection groups.

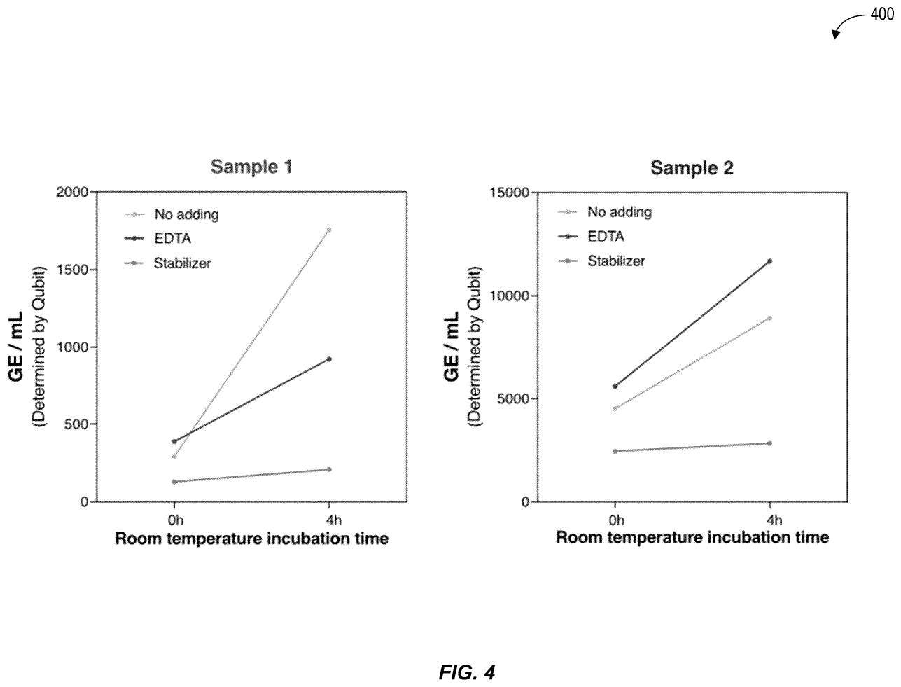

FIG. 4 shows a set of graphs 400 that show comparisons of urinary cell-free DNA concentrations comparisons between before and after in-vitro incubation among three urine collection groups (control group without adding any stabilizer, EDTA, and stabilizer groups). The term “GE” refers to genome equivalent. As shown in FIG. 4, the fold changes of the urinary cell-free DNA concentration after 4-hour incubation versus at 0-hour incubation were the lowest in the stabilizer group (Sample 1: 1.61 GE/ml; Sample 2: 1.15 GE/ml) when compared with that in the control group without adding any stabilizer (Sample 1: 6.04 GE/ml; Sample 2: 1.98 GE/ml) and EDTA group (Sample 1: 2.37 GE/ml; Sample 2: 2.09 GE/ml).

The autosomal DNA size profiles were further compared before and after in-vitro incubation among three collection groups.

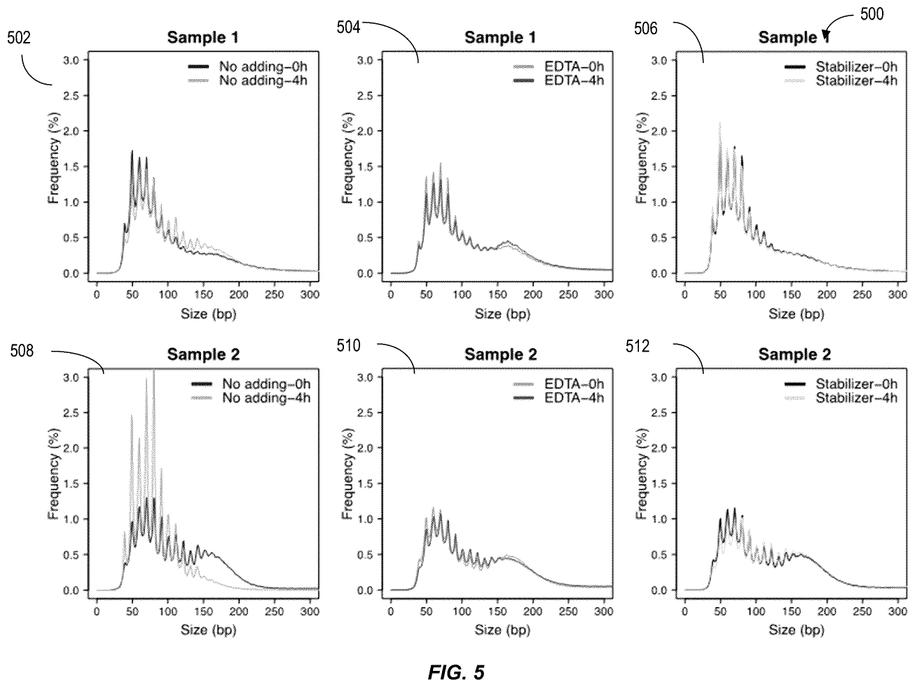

FIG. 5 shows a set of graphs 500 identifying comparisons of urinary cell-free DNA size profiles before and after in-vitro incubation among control, EDTA, and stabilizer groups. The first set of graphs 502, 504, and 506 correspond to cell-free DNA size profiles for a first sample, and a second set of graphs 508, 510, and 512 correspond to cell-free DNA size profiles for a second sample. In the control group (the graph 502, the graph 508), the urinary cell-free DNA size profiles changed greatly after 4-hour incubation compared to 0-hour incubation. In the EDTA group (the graph 504, the graph 510), the size profile of urinary cell-free DNA changed a little after 4-hour incubation compared with 0-hour incubation. In the stabilizer group (the graph 506, the graph 512), the urinary cell-free DNA size profile showed the least change (i.e. barely observable) after 4-hour incubation compared with 0-hour incubation. The results shown in FIG. 5 suggested that the use of the device containing a stabilizer could optimally preserve the fragmentation profile of urinary cell-free DNA molecules under the condition of room temperature.

### C. Cell-Free DNA Extraction and Library Preparation

As shown in FIG. 3, cell-free DNA can be extracted from the urine (treated with stabilizers) with Wizard Plus Minipreps DNA Purification System (Promega) and guanidine thiocyanate (Sigma-Aldrich). Cell-free DNA was extracted from the plasma samples with the QIAamp Circulating Nucleic Acid Kit (Qiagen) according to the manufacturer's protocol. Indexed DNA libraries were constructed using a TruSeq DNA Nano Library Prep Kit (Illumina) according to the manufacturer's instructions. The adaptor-ligated DNA was enriched with by PCR and then analyzed on Agilent 4200 TapeStation (Agilent Technologies) for quality control and gel-based size determination. Libraries were quantified by the Qubit dsDNA high sensitivity assay kit (Thermo Fisher Scientific) before sequencing.

### D. DNA Sequencing and Alignment

As further shown in FIG. 3, multiplexed DNA libraries were sequenced for paired-end reads on the Illumina platform. Other sequencing techniques may be used, e.g., as described herein. For example, a single read for an entire DNA fragment can be determined. Sequences were assigned to their corresponding samples based on their six-base index sequence. Using the Short Oligonucleotide Alignment Program 2 (SOAP2), the paired-end reads from mouse plasma were aligned to the mouse reference genome (NCBI build 37/UCSC mm9; non-repeat-masked) or human reference genome (NCBI build 37/hg19) (Li et al. Bioinformatics 2009; 25:1966-1967). Any other alignment tool may also be used, as will be appreciated by the skilled person. In some implementations, up to two nucleotide mismatches were allowed. Only paired-end reads aligned to the same chromosome in the correct orientation and spanning an insert size of <600 bp can be retained for downstream analysis. Paired-end reads sharing the same start and end genomic coordinates were deemed PCR duplicates and were discarded from downstream analysis.

For some use cases, e.g., for plasma samples, genotype of the buffy coat DNA from mother can be paired with corresponding placenta samples. In effect, maternal and fetal genotype can be determined. The genotypes can be used to differentiate the fetal and maternal DNA molecules, such that we can obtain the gold standard for fetal DNA fractions in urine samples. This actual fetal DNA fraction would also allow us to establish the recalibration curve for estimating the degree of transrenal DNA or the kidney permeability assuming the higher the kidney permeability, the more transrenal DNA.

### III. SIZE CHARACTERISTICS OF URINARY CELL-FREE DNA

The size characteristics of urinary cfDNA were analyzed to illustrate the effect of fragment size on the ability of transrenal cfDNA fragments to pass through the kidney into urine. Smaller-sized molecules are shown to have an increased ability to pass through the kidney from the blood.

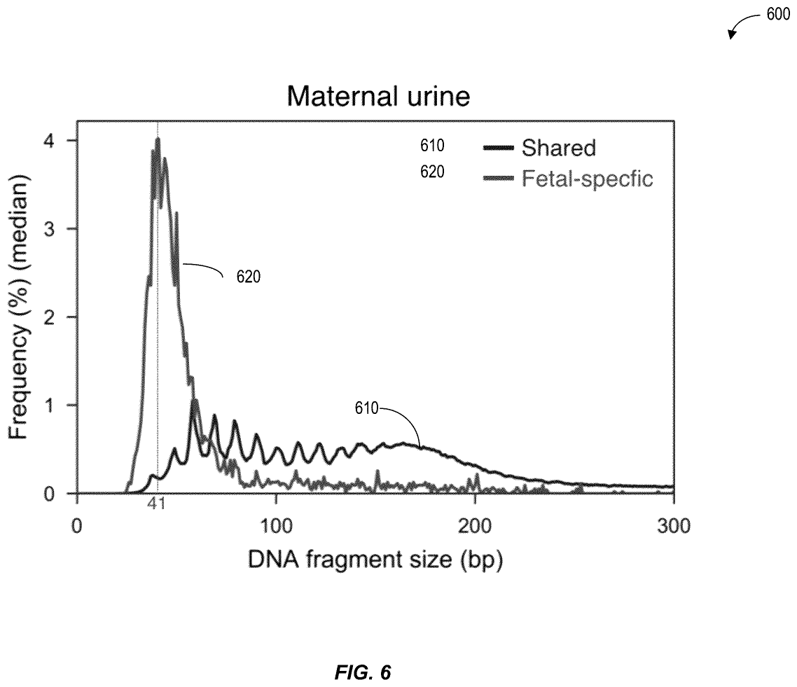

FIG. 6 shows a graph 600 that identifies size differences between fetal DNA and maternal DNA in urine samples, according to some embodiments. The size differences between fetal DNA and maternal DNA can be used as examples for determining transrenal DNA in urine samples. For example, the fetal DNA originates from the fetus and has to pass through the kidney so that we can detect them in urine. In contrast, the detected maternal DNA can include non-transrenal DNA, which can be contributed by kidney, bladder, etc. Based on this distinction, we can extend the characteristics of fetal and maternal DNA to determine size differences between transrenal and non-transrenal DNA.

As shown in FIG. 6, the majority of the fetal-specific cell-free DNA molecules 620 (red) were shown to be less than 80 bp, which was substantially shorter than shared cell-free DNA molecules 610 (blue). The shared cell-free DNA molecules 610 have a same allele that is shared among haplotypes of the mother and one haplotype of the fetus. The fetal-specific cell-free DNA molecules 620 have a fetal-specific allele (inherited from the father) that is on one of the fetal haplotypes.

Based on the above, it can be considered whether size characteristics of transrenal DNA can be correlated with those of fetal DNA.

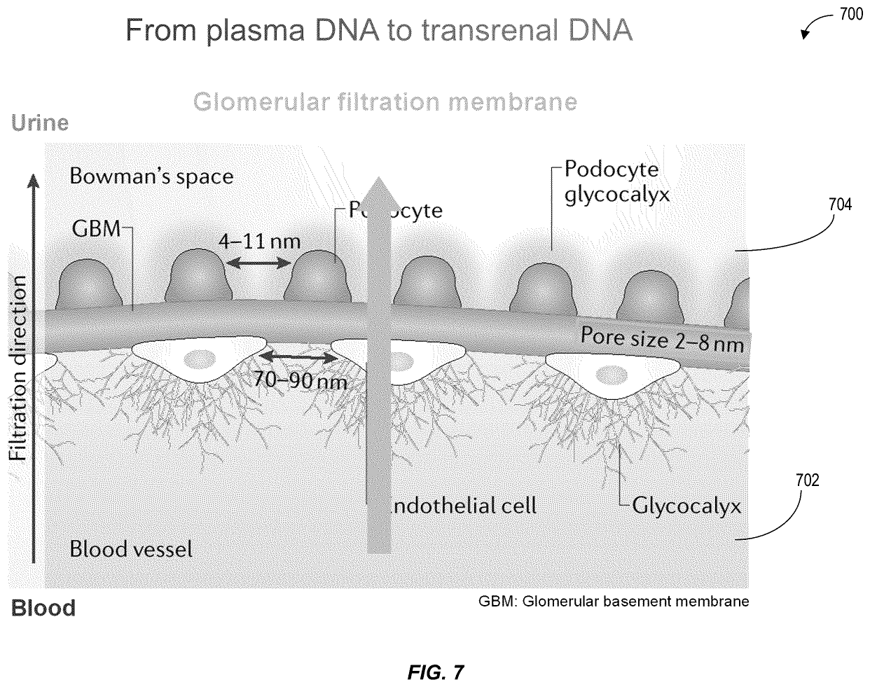

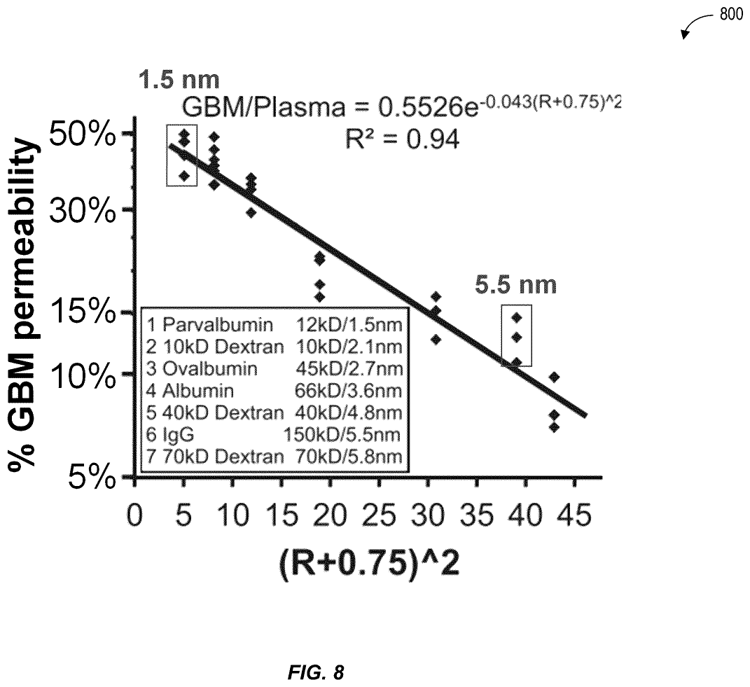

FIG. 7 illustrates an example schematic diagram 700 that shows a biological process of converting plasma DNA into transrenal DNA, according to some embodiments. For example, in order to become transrenal DNA, the plasma DNA from blood vessels 702 go through various tissue membranes to reach the kidney 704. For example, the plasma DNA molecules pass through the endothelial cells and the glomerular basement membrane (GBM), as well as the podocyte. As shown in FIG. 7, each of these biological structures has a different diameter. The kidney structures can be associated with pore sizes of the kidney through which the plasma DNA molecules pass to become transrenal DNA. It can be considered that the plasma DNA molecules that are sufficiently small to pass through the pores can eventually become transrenal DNA.

FIG. 8 shows a graph 800 that identifies a relationship between glomerular basement membrane permeability and sizes of transrenal DNA, according to some embodiments. The permeability of the glomerular basement membrane can be determined by the molecular size. As shown in FIG. 8, the x-axis is the radius of a given molecule, which corresponds to its size. And, the y-axis identifies kidney permeability associated with the given molecules.

In particular, the % GBM permeability indicated in y-axis identifies a percentage of molecules having a particular size would pass through the GBM. For example, if the molecule is very small (e.g., 12 kDa), the permeability is estimated to be approximately 50%. In contrast, as the molecule becomes larger (e.g., 150 kDa), the kidney permeability will drop significantly to around 10 to 15%. It is also known that a nucleosome typically has a size of 200 kDa/5.5 nm (radius). Based on the size of the nucleosome, it can be hypothesized DNA molecules that are wrapped in nucleosomes (thus attached to proteins) would be associated with low GBM permeability compared to nucleosome-depleted DNA molecules. In effect, the sizes of transrenal DNA molecules that pass through the GBM would likely have smaller sizes compared to non-transrenal DNA molecules which originate directly from the urinary system.

### IV. END-MOTIF CHARACTERISTICS OF URINARY CELL-FREE DNA

In addition to size, ending sequences of urinary cell-free DNA were analyzed to determine that the end motifs of transrenal urinary cell-free DNA molecules differed from end motifs of non-transrenal urinary cell-free DNA molecules. In some embodiments, the 4-mer end motif is defined as the terminal 4 nucleotides at each 5′ fragment end of cell-free DNA molecules, totaling 256 categories of 4-mer end motifs (i.e., 4⁴). The median end motif frequencies of 256 end motifs were calculated and ranked in descending order for fetal-specific and shared fragments in maternal urine samples separately. Other end motifs may be used, e.g., any K-mer end motif, e.g., with K being 1, 2, 3, 4, 5, 6, 7, 8, 9, or more. As described herein, end motifs (e.g., sample end-motif profiles and reference profiles, referred to as reference F-profiles) can be used in various ways to determine a property of a urine sample and/or a classification of a subject, such as determining a fractional concentration of clinically-relevant DNA, a gestational age of a fetus, or a level of pathology of a subject.

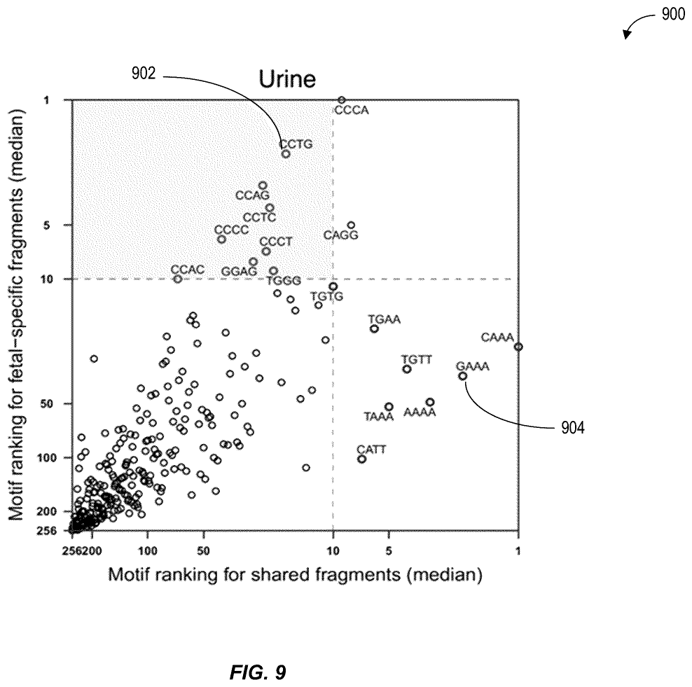

FIG. 9 shows a graph 900 that identifies different end-motifs identified from fetal-specific and shared cell-free DNA in maternal urine. As shown in FIG. 9, the median end-motif frequencies of 256 end motifs in fetal-specific shared cell-free DNA were calculated and ranked in descending order in maternal urine samples. The x-axis identifies the end-motif rankings of the shared fragments. The y-axis identifies the motif ranking for fetal-specific fragments. A higher ranking indicates relative frequencies being higher for a corresponding end motif (e.g., CCTG). The colored areas respectively show fetal-specific or shared DNA fragments having a preference for specific 4-mer end motifs.

Each of the top 10 motifs in both fetal-specific and shared cell-free DNA was labeled with a corresponding end-motif sequence. The top 10 motifs for fetal-specific and shared urinary cell-free DNA were highlighted by red circles 902 and blue circles 904, respectively. Top 10 end motifs for fetal-specific cell-free DNA were predominated by C-end motifs (8/10), while top 10 end motifs for shared cell-free DNA were enriched for T-end motifs (4/10). It has been previously identified that DNASE1L3 (which prefers to cut C) is the dominant nuclease in plasma, while DNASE1 is (which prefers to cut T) the dominant nuclease in urine. Based on the above motif rankings, it can be determined that the fetal DNA corresponds to transrenal DNA. The data suggested that one could use motifs containing C-ends to represent the transrenal urinary cell-free DNA, which can then be used to differentiate fetal DNA from maternal urine samples. As described below, some embodiments can use the existence of a C at the end of a cfDNA molecule to enrich a sample for clinically-relevant DNA, e.g., all transrenal DNA, fetal DNA, tumor DNA, or DNA from a particular tissue other than kidney or bladder.

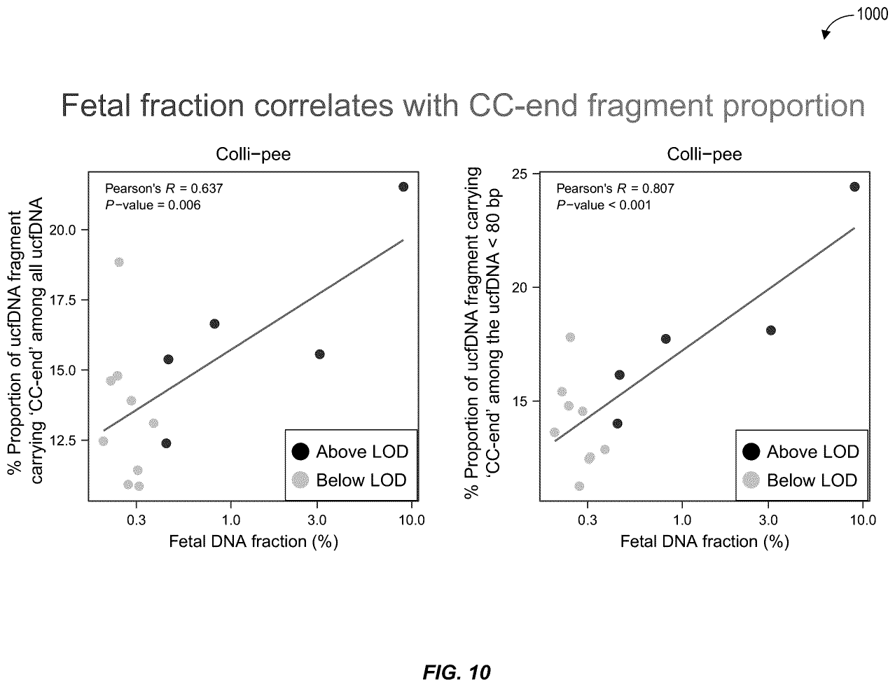

FIG. 10 shows a set of graphs 1000 that identify a relationship between fractional concentration of fetal DNA with CC-end fragments in urine samples, according to some embodiments. As shown in FIG. 10, the percentage of urinary cell-free DNA carrying CC-ends increase in proportion to the fetal DNA fraction in the urine samples. This linear relationship is further pronounced when the CC-fragments were limited to fragments having sizes less than 80 base pairs. Thus, the end-motifs of urine samples can be used for determining fraction of fetal DNA.

### V. ANALYSIS OF URINARY CELL-FREE DNA FOR OCRS

Open chromatin regions can be used to determine a property of a urine sample and/or a classification of a subject. In some circumstances, the open chromatin regions can be associated with tissues contributing to transrenal DNA or even a particular cell type tissue (e.g., fetal, tumor, transplanted organ, or other tissue, such as blood, liver, colon, etc., besides the tissues from urinary tract). For example, an abundance of cfDNA from such a set of regions can be used as part of estimating fractional concentration of clinically-relevant DNA, determining a classification of a pathology, such as a kidney abnormality, or detecting preeclampsia or proteinuria, which can be classified as types of a kidney abnormality.

Permeability of kidney membranes (e.g., GBM) favors shorter DNA fragments. As a result, transrenal cell-free DNA that pass through the kidney membranes are shorter than non-transrenal DNA fragments that originate directly from the urinary system. In addition, cell-free DNA molecules that are bound to nucleosomes can have difficulties in passing through the GBM, as permeability of nucleosomes are estimated to be about 10-15%. By contrast, nucleosome-depleted cell-free DNA molecules originating from open chromatin regions are not bound by any nucleosomes and may pass through the GBM with higher permeability. Based on the above characteristics, identifying cell-free DNA molecules originating from open chromatin regions can be used to detect transrenal DNA in urine samples. In addition, the contribution of cell-free DNA molecules from open chromatin regions can also be used to predict classification of certain diseases as well as determine fractional concentration of fetal DNA.

A. Correlation Between Transrenal DNA and DNA from OCRs

Transrenal DNA can be correlated with DNA of open chromatin regions, according to some embodiments. The nucleosome-depleted cell-free DNA molecules in plasma have smaller molecular sizes, which allow them to pass through the GBM and transform into transrenal DNA. Based on this characteristics, it can be determined whether the transrenal DNA is enriched in open chromatin regions, which corresponds to nucleosome depleted regions. Such enrichment is described in later sections, e.g., for all transrenal DNA or for certain tissue types.

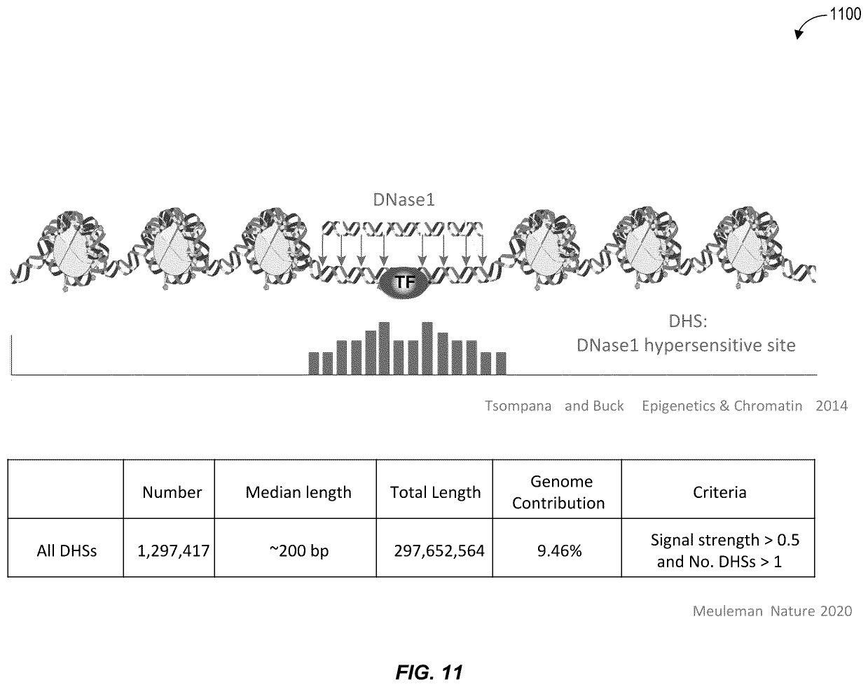

FIG. 11 illustrates an example diagram 1100 that identifies various characteristics of DNA molecules from open chromatin regions, according to some embodiments. The open chromatin regions can be identified in various ways, e.g., based on locations of DNase1 hypersensitive sites (DHS), since DNase1 has a cutting preference within genomic regions being relatively in lack of bound histones. For example, a given open chromatin region can be identified as a genomic region clustered with DNase1-digested fragment ends. There are approximately 1 million DHS sites, and the median length of the regions are around 200 base pair. Such open chromatin regions contribute to 9.46% of the genome. Such DHSs (and corresponding OCRs) can be specific to a particular tissue, e.g., placenta-specific DHSs.

As another example for identifying OCRs, to obtain DNA molecules from the open chromatin regions, DNase-Seq can be used. Specifically, DNA molecules from a urine sample can be digested using DNase1, at which DNA molecules having the hypersensitive sites are preferentially cut and sequenced. The sequence reads can then be considered as DNA molecules from open chromatin regions. As further examples, OCRs can identified from but not limited to sites identified using DNase-seq, sites identified using Assay for Transposase-Accessible Chromatin using sequencing (ATAC-seq), and transcriptional start sites (TSS).

### B. Identifying Tissue-Specific OCRs

OCRs can be determined and used in general for all tissues, for ones specific to tissue that is transrenal, or for specific tissues. Different tissues generally have different regions that are open chromatin in them. Thus, a specific set of OCRs can be identified, e.g., depending on what the desired clinically-relevant DNA is. As an example, whether a class of cfDNA is enriched or depleted in one or more OCRs can be determined in the following matter, e.g., to identify an OCR associated with one or more tissues.

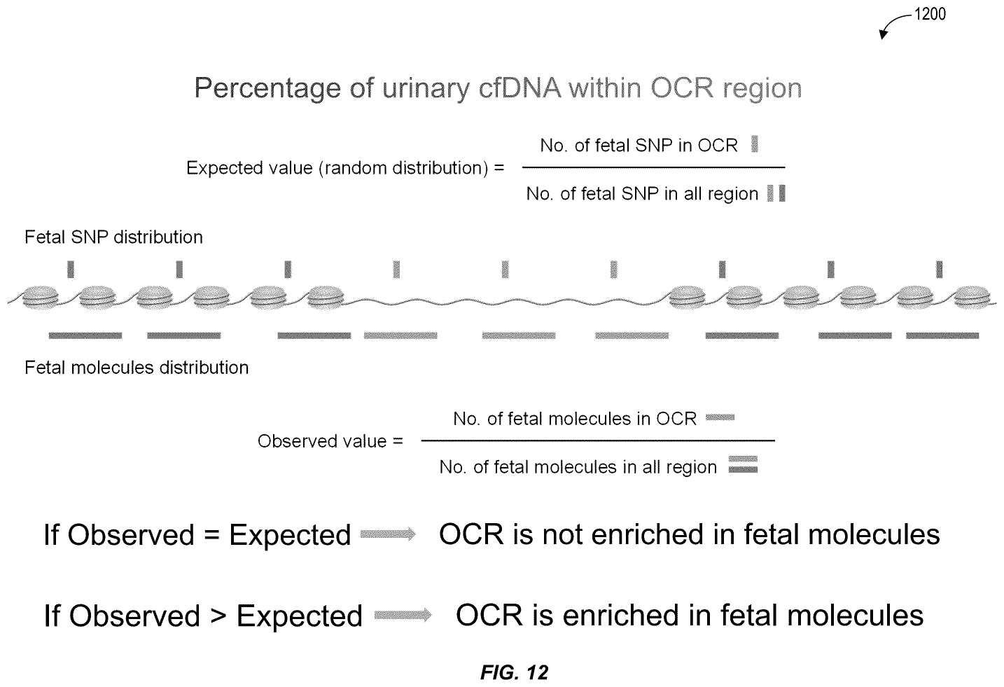

FIG. 12 illustrates an example diagram 1200 for determining amounts of urinary cell-free DNA corresponding to open chromatin regions, according to some embodiments. To analyze the fragmentomic features based on urinary cell-free DNA from open chromatin regions, we collected 14 pregnant women's urine samples and studied the fetal cell-free DNA properties in maternal urine. The characteristics of fetal-specific cell-free DNA molecules and shared cell-free DNA molecules can represent the characteristics of transrenal cell-free DNA and non-transrenal cell-free DNA, respectively. Such representation can be made due to the transrenal cell-free DNA being shorter than the non-transrenal cell-free DNA.

In the example shown in FIG. 12, cfDNA having any fetal-specific allele (e.g. a single nucleotide polymorphism, SNP) can be identified. Then, the amounts of such cfDNA in windows (e.g., 10, 20, 30, 40, 50, or 60 bp) across the genome can be determined by aligning the sequence reads to a reference genome. An expected value can be determined as a number of fetal-specific SNPs in a region (where a region can include one or more windows) divided by an average or median of fetal-specific SNPS for all windows/regions. An observed value can be determined as a number of fetal-specific reads in a region divided by an average or median for all windows/regions. If the observed ratio is greater than the expected ratio, then the region can be identified as an OCR, since the smaller fetal DNA fragments are more prevalent. Such OCRs determined using fetal DNA would be specific to fetal tissue. But DHS sites for various tissues can be used or more generally OCRs for various tissues. Thus, DHS of a particular tissue or set of tissues can be used.

In an example implementation, to assess transrenal urinary cell-free DNA in pregnant women, we obtained a median of fetal fraction of 0.31% in maternal urine samples (range: 0.20%-9.00%). Fetal DNA fractions were estimated by a single nucleotide polymorphism (SNP)-based approach in maternal urine cell-free DNA (Yu et al. Clin Chem. 2013; 59:1228-1237). The contribution of nucleosome-depleted cell-free DNA can be indicated by an amount of DNA molecules derived from the open chromatin regions (OCR), in which the OCR correspond to nucleosome-deleted regions (i.e. a lack of histone-bound DNA). For illustration purposes, we use the DNase1 hypersensitive sites (DHS) defined from DNase-seq to represent the OCR (Meuleman et al. Nature. 2020; 584:244-251).

From the OCRs, the amount of nucleosome-depleted DNA molecules was determined by the number of sequenced cell-free DNA molecules that were aligned to the OCR. The amount of nucleosome-depleted DNA molecules can be a normalized value. For example, the amount of nucleosome-depleted DNA molecules can be translated into percentages by dividing the total sequenced molecules. Additionally or alternatively, the amount of nucleosome-depleted DNA molecules could be calculated by the observed sequenced molecules within OCR (O) (also referred to as observed OCR-related DNA contribution) divided by the expected OCR-related value (E). This measurement is herein defined as “O/E ratio.”

The expected OCR-related DNA contribution can correspond to a theoretical percentage of OCR in a reference genome. For example, the observed value (O) can include the percentage of the fragments aligned to OCR in all fragments and the expected value (E) as the theoretical percentage of OCR in the reference genome (e.g., a human reference genome). In some instances, the expected OCR-related DNA contribution for fetal-specific DNA can be calculated by the number of fetal-specific single-nucleotide polymorphisms (SNPs) in OCR normalized by the number of fetal-specific SNPs in all genomic regions. Such a relative frequency (e.g., a percentage) can provide the expected percentage, which can be compared to the observed percentage of cell-free DNA molecules aligned to the OCRs. The SNPs can be obtained through the genotyping analysis. In some instances, the expected OCR-related DNA contribution corresponds to a percentage of DNA molecules falling within OCRs by random sampling.

The observed OCR-related DNA contribution in fetal DNA can be calculated by the number of fetal-specific molecules aligned to OCR normalized by the number of fetal-specific molecules aligned to all genomic regions. For the O/L ratio analysis in non-transrenal urinary cell-free DNA, molecules carrying the shared alleles between the fetal and maternal genomes were analyzed according to embodiments in the present disclosure. To use the O/L ratio to determine OCR enrichment, if the O/L ratio was close to 1, no OCR enrichment is found. If the O/L ratio was greater than 1, OCR-related DNA contribution was increased. A higher O/E ratio could be suggestive of a higher nucleosome-depleted DNA contribution, likely indicating a higher glomerular permeability. Regions specific to other tissues can be identified in a similar manner.

C. Quantifying Transrenal cfDNA from OCRs for Urine and Plasma

The amount of cfDNA in OCR regions can be used to quantify transrenal DNA in a urine sample. Fetal cfDNA is used as an example of transrenal DNA, but other examples would apply to tumors and other tissues that produce transrenal DNA.

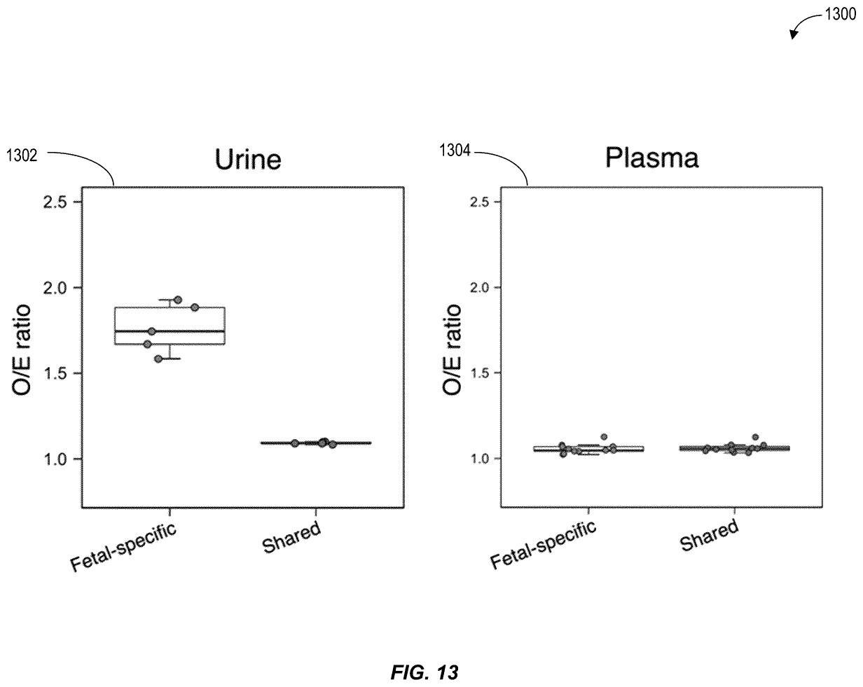

FIG. 13 shows a set of boxplots 1300 of O/E ratios of fetal-specific and shared cell-free DNA molecules in urine samples (boxplot 1302) and plasma samples (boxplot 1304) in open chromatin regions. These OCRs were not tissue-specific but instead were a general sampling of OCRs across different tissues. Specifically, all known DHS sites were used.

As shown in boxplot 1302, the median O/E ratio of fetal-specific cell-free DNA that was transrenal urinary cell-free DNA was 1.84 (range: 1.68-2.13), which was 1.67-fold higher than that of shared cell-free DNA mainly of non-transrenal origin (median: 1.10; range: 1.08-1.19) in urine samples with fetal fraction above 0.44%. In contrast, no obvious enrichment in O/E ratio was found in both fetal-specific (median of O/E ratio: 1.048; range: 1.023-1.126) and shared cell-free DNA (median: 1.058; range: 1.033-1.124) in plasma samples (the boxplot 1304).

Taken together, the OCR-related DNA contribution was elevated in fetal DNA (an example of transrenal DNA molecules), compared with non-transrenal DNA molecules. These data indicated that one could use the amount of OCR-related DNA to estimate the fractional concentration of transrenal cell-free DNA in urine. The higher the O/E ratio the higher the fractional concentration of the clinically-relevant DNA, e.g., the one or more tissues for whose OCRs were used. When OCRs of different transrenal tissues are used, the fractional concentration will correspond to an average concentration (e.g., weighted by how many and size of corresponding OCRs) of those tissues. The fractional concentration can approximate the transrenal DNA concentration, where more OCRs of different tissues can provide increased accuracy for approximating the transrenal DNA concentration.

To estimate the fractional concentration, calibration (training) samples having a known fractional concentration of the clinically-relevant DNA can be used. A calibration value can correspond to the relative abundance for a calibration sample, where the calibration value and the known fractional concentration comprise a calibration data point. If a new sample has a higher relative abundance, then the new sample has a higher fractional concentration then the calibration sample. If a new sample has a lower relative abundance, then the new sample has a lower fractional concentration then the calibration sample. Using multiple calibration samples, a range for a fractional concentration can be determined. In other implementations, a calibration function (also referred to as a calibration curve) can be determined via a functional fit (e.g., linear or non-linear regression) of the calibration data points.

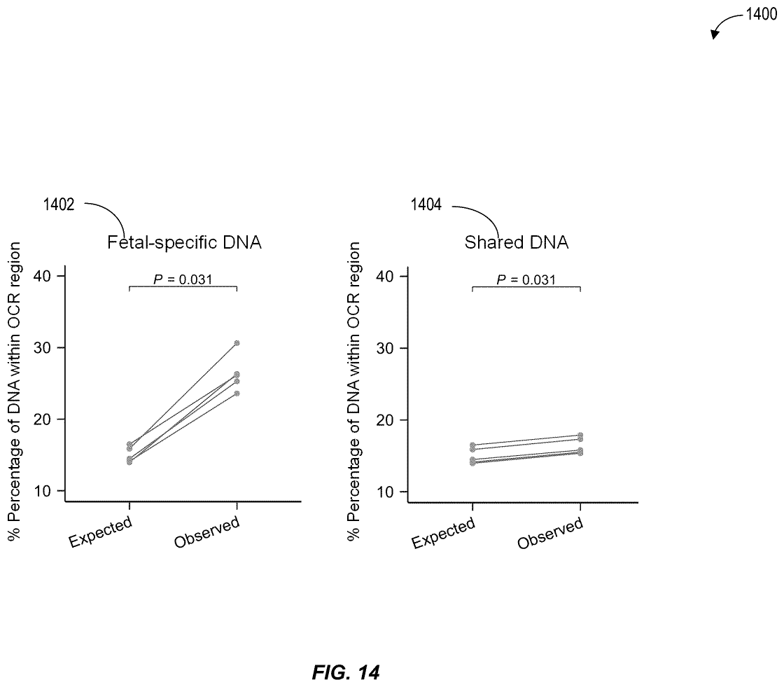

FIG. 14 illustrates a set of graphs 1400 that identify enrichment of DNA molecules in open chromatin regions in fetal-specific DNA from urine samples, according to some embodiments. Graph 1402 identifies expected and observed percentages of fetal-specific urinary DNA molecules that are from OCR regions. Graph 1404 identifies expected and observed percentages of shared urinary DNA molecules that are from OCR regions. The expected can be determined based on a size of the OCR regions as a proportion of the genome.

As shown in the graph 1402, fetal-specific urinary DNA is enriched in the open chromatin regions, as the observed values are substantially greater than expected values. Further, the graph 1404 shows that the expected and observed values for the shared urinary DNA molecules indicate a smaller decrease. Based on the graphs 1402 and 1404, it is shown that urinary DNA is enriched in the open chromatin regions. These results also suggest that filtration mechanisms of the kidney contribute to an enrichment of transrenal DNA in open chromatin regions. Accordingly, embodiments can enrich a urine sample for clinically-relevant DNA, e.g., by selecting cfDNA from open chromatin regions specific to one or more transrenal tissues (e.g., fetal, tumor, transplant, or transrenal tissue in general).

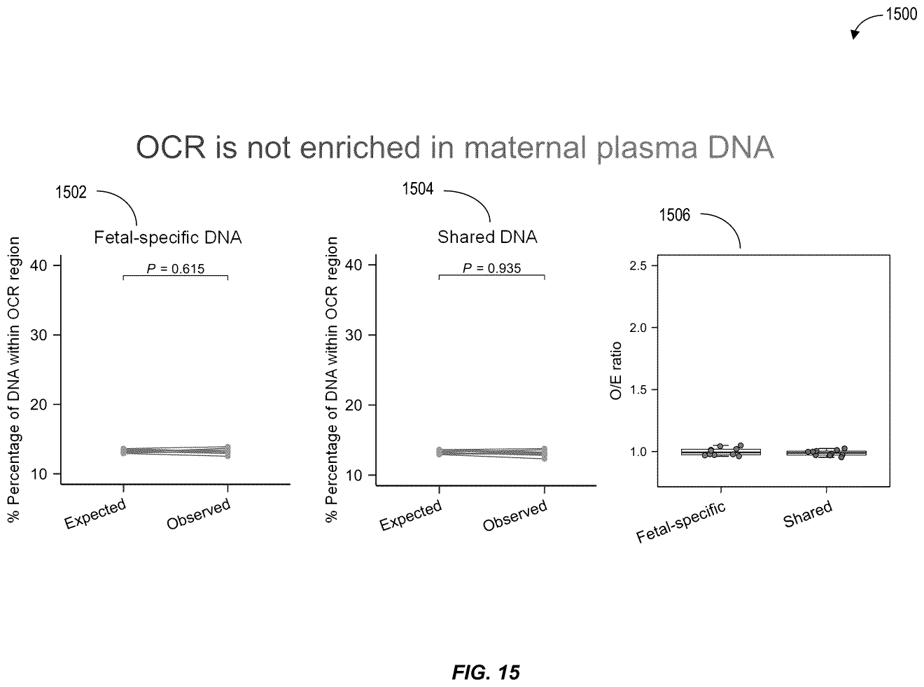

FIG. 15 illustrates a set of graphs 1500 that identify non-enrichment of DNA molecules in open chromatin regions in fetal-specific DNA from plasma samples, according to some embodiments. Graph 1502 identifies expected and observed percentages of fetal-specific plasma DNA molecules that are from OCR regions. Graph 1504 identifies expected and observed percentages of shared plasma DNA molecules that are from OCR regions. As shown in the graphs 1502 and 1504, both fetal-specific plasma DNA and shared plasma DNA are not enriched in the open chromatin regions. In addition, the O/E ratios shown in graph 1506 also suggest that there is no enrichment of plasma DNA from open chromatin regions. Accordingly, the observed enrichment of urinary cell-free DNA in open chromatin regions can be used to determine a fraction of fetal-specific DNA, whereas such determination would not be feasible for plasma cell-free DNA.

### VI. IDENTIFYING CLINICALLY-RELEVANT DNA IN URINE

As the differential fragmentation patterns between transrenal and non-transrenal urinary cell-free DNA can be identified, we hypothesized that transrenal cell-free DNA contribution could be enriched by selectively analyzing fragmentomic features of transrenal urinary cell-free DNA. The fragmentomic features can include, but are not limited to, end motif (e.g., CC-ends), genomic regions (e.g., OCR), and size (e.g., <=80 bp). Moreover, the accuracy of determining transrenal cell-free DNA contribution can be further enhanced by identifying urinary cell-free DNA molecules that are from open chromatin regions.

A. Estimating Amount of Clinically-Relevant DNA Using Abundance from OCRs

As previously shown in FIG. 13, the greater OCR-related DNA enrichment was observed in fetal-specific urinary cell-free DNA compared with shared cell-free DNA in urine samples. This is different from non-enrichment of open chromatin regions for plasma DNA molecules.

Accordingly, fetal DNA fraction (or fraction of other clinically-relevant DNA) can be determined in urine samples using the O/E ratio of all urinary cell-free DNA fragments. To calculate the O/E ratio of all urinary cell-free DNA fragments, observed OCR-related DNA contribution can be determined as the percentage of the fragments aligned to OCR in all fragments. Expected OCR-related DNA contribution can be defined as the theoretical percentage of OCR in a reference genome (e.g., a human reference genome). 1. O/E ratios

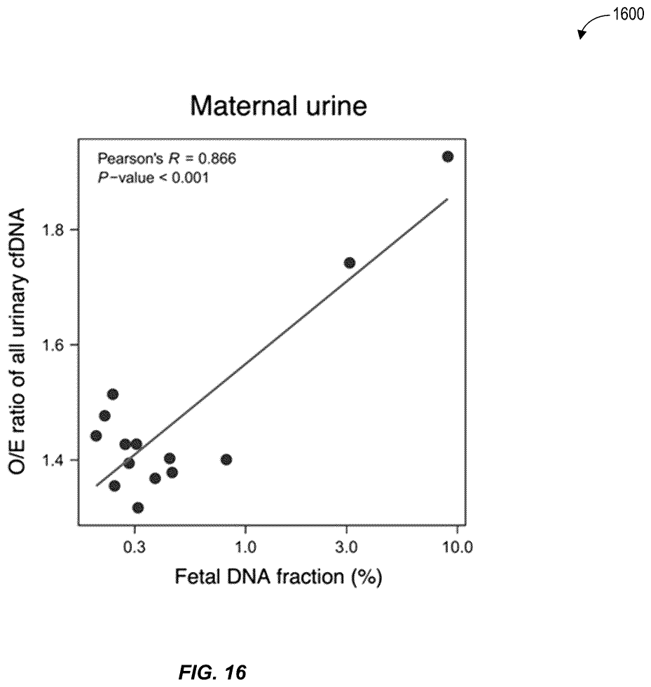

FIG. 16 shows a graph 1600 that identifies a correlation between fetal DNA fraction in maternal urine and the O/E ratio of all urinary cell-free DNA fragments from OCR regions. All OCRs corresponding to DHS sites were used. Here, we define the observed value (O) as the percentage of the fragments aligned to OCR in all fragments and the expected value (E) as the theoretical percentage of OCR in the reference genome. The higher the O/E ratio, the more fragments are enriched from OCR regions. As shown in FIG. 16, the fractional concentration of fetal DNA in maternal urine increased in proportion to the O/E ratio of all urinary cell-free DNA fragments (Pearson's R=0.866; P-value <0.001). Thus, fractional concentration of fetal DNA can be estimated in urine samples by determining the O/E ratios of DNA molecules that are from OCRs.

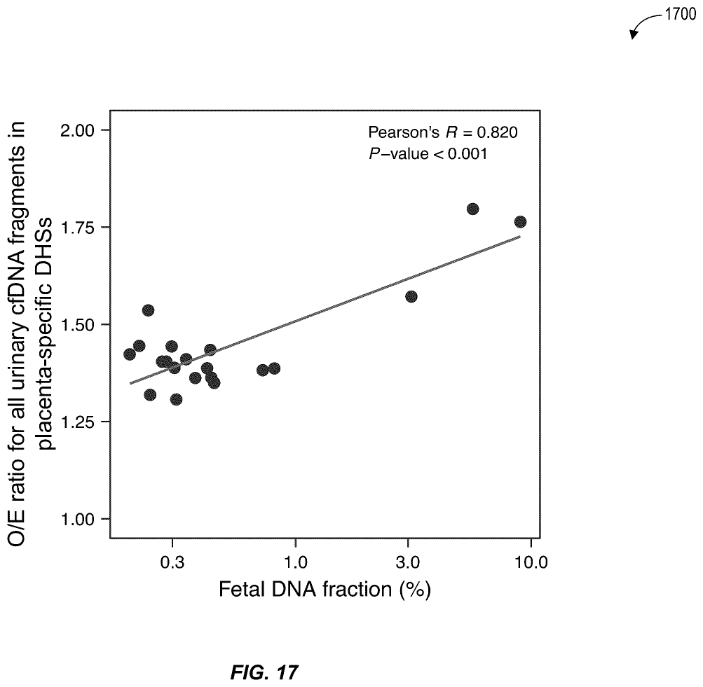

FIG. 17 shows a graph 1700 illustrating a correlation between fetal DNA fraction in maternal urine and the O/E ratio of urinary cfDNA fragments from placenta-specific DHSs. As when using all DHS sites, the higher the O/E ratio, the more fragments are enriched from OCR regions. As shown in FIG. 17, the fractional concentration of fetal DNA in maternal urine increased in proportion to the O/E ratio of all urinary cell-free DNA fragments in placenta-specific DHSs (Pearson's R=0.820; P-value <0.001). Thus, fractional concentration of fetal DNA can be estimated in urine samples by determining the O/E ratios of DNA molecules that are from tissue-specific OCRs, e.g., placenta-specific OCRs.

### 2. Normalized End Density and Use of Size

As another example of using relative abundance to determine a fractional concentration of clinically-relevant DNA, the end density of overall urinary cell-free DNA located in OCR is used for determining fetal fraction in urine samples. The end density can identify a rate of DNA molecules ending on a particular position (e.g., DNase1 hypersensitive sites). For example, for every DNase1 hypersensitive site, the normalized end density can be calculated at 0 bp distance to the central genomic location. The higher normalized end density at OCR (0 bp distance to the central genomic location of OCR) can be associated with a higher fraction of transrenal cell-free DNA (e.g., fetal DNA) in urine.

To determine the end density at the OCR regions, we analyzed 14 maternal urine samples and 11 maternal plasma samples. Both 5′ and 3′ ends of the DNA fragment within the 1-kb upstream and 1-kb downstream of the central genomic location of OCR were analyzed. The normalized end density was defined as the count of fragment ends located within a window (e.g., 1-kb upstream and 1-kb downstream) around an OCR divided by the median or mean count across loci/regions neighboring (e.g., flanking) one or more of the OCRs used. Other windows can also be used upstream or downstream, e.g., at least 100, 200, 300, 400, 500, 600, 700, 800, 900, or greater than 1,000 bp. As examples, the neighboring loci can be outside the window used to define the OCR and can be of various lengths, e.g., as recited above.

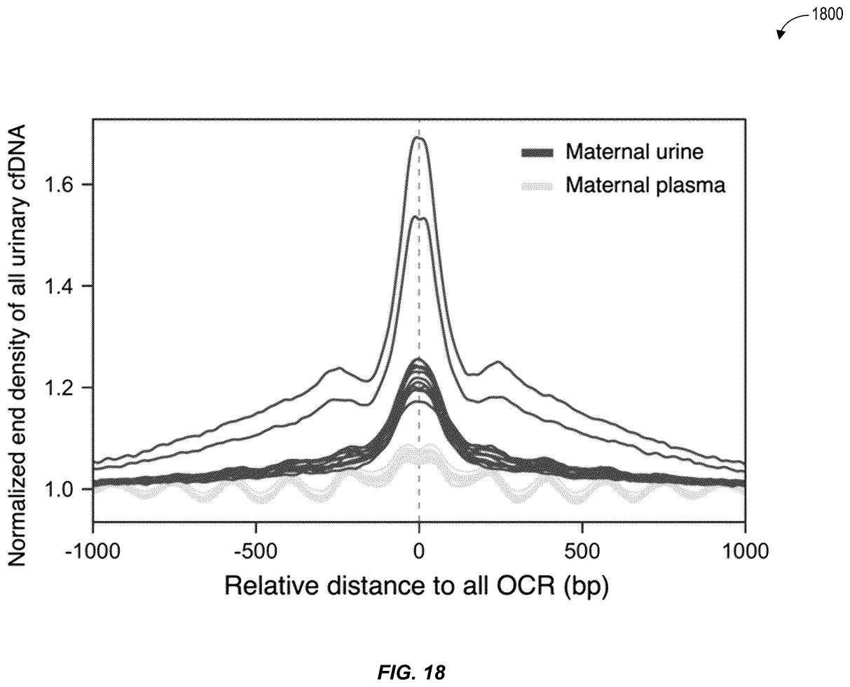

FIG. 18 shows a graph 1800 that identifies the normalized end density of all urinary cell-free DNA in OCR of maternal urine and plasma samples. All OCRs corresponding to DHS sites were used. The maternal urine and maternal plasma samples were indicated with red lines and yellow lines, respectively. As shown in the graph 1800, the normalized end density for OCRs was substantially more enriched in maternal urine samples than that in maternal plasma samples.

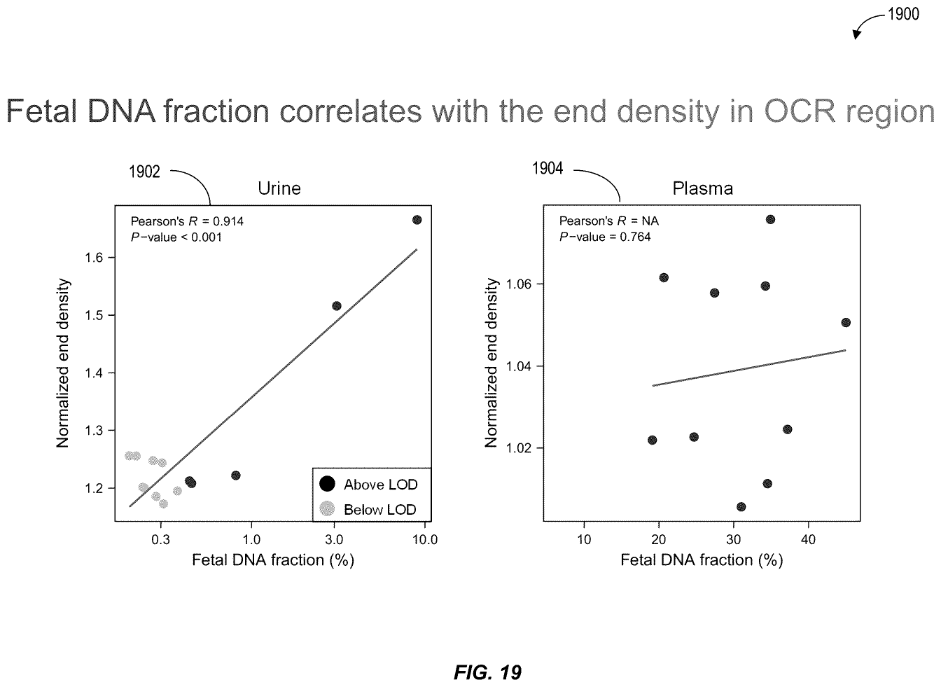

FIG. 19 shows a set of graphs 1900 that identify a comparison between end density of urinary cell-free DNA and plasma cell-free DNA for determining fractional concentration of fetal DNA, according to some embodiments. For urine samples, the end density of DNA molecules from OCRs can be used to determine fractional concentration of fetal DNA. Such determination cannot be performed for plasma samples. Graph 1902 shows that the fetal DNA fraction increased in proportion to the normalized end density of urinary cell-free DNA, whereas graph 1904 does not show such increase for plasma cell-free DNA. Thus, the relative abundance at OCRs in a urine sample can be used to estimate a fractional concentration of clinically-relevant DNA in the urine sample.

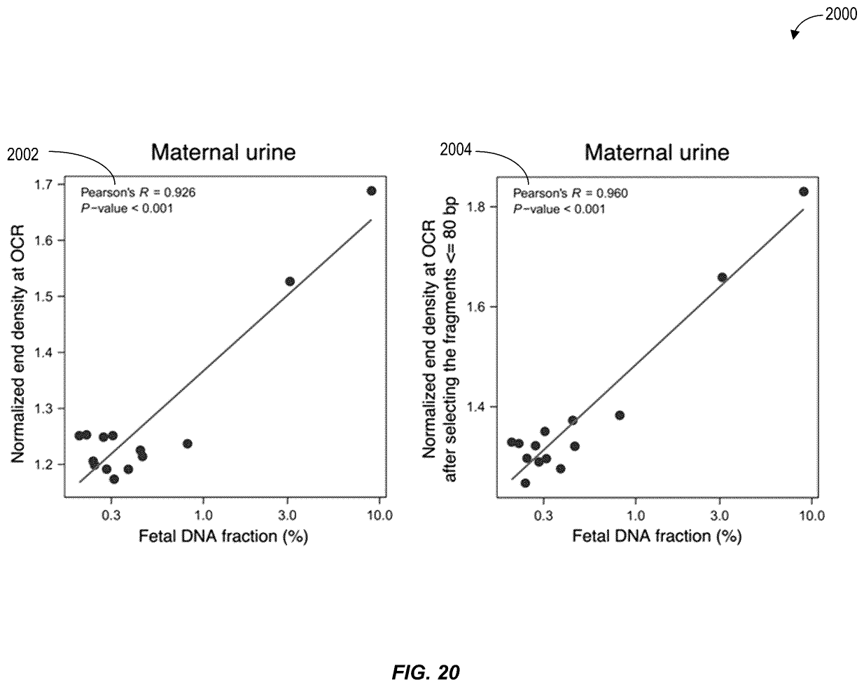

FIG. 20 shows a set of graphs 2000 that identify correlations between fetal DNA fraction and the normalized end density of urinary cell-free DNA having different sizes, according to some embodiments. In FIG. 20, a graph 2002 shows normalized end density of all cell-free DNA fragments in maternal urine samples, and a graph 2004 shows normalized end density of fragments that have sizes equal or less than 80 bp in the maternal urine samples. Other size thresholds (size cutoffs) can be used besides 80 bp, as is described herein.

In the graph 2002, the fetal DNA fraction in maternal urine was significantly correlated with the normalized end density at OCR (Pearson's R=0.926; P-value <0.001). The correlation between fetal DNA fraction in maternal urine and the normalized end density at OCR could be further enhanced by selecting the fragments <=80 bp (Pearson's R=0.960; P-value <0.001) (the graph 2004). The results suggested that the use of molecules derived from OCRs could inform the extent of transrenal urinary cell-free DNA.

### 3. Method

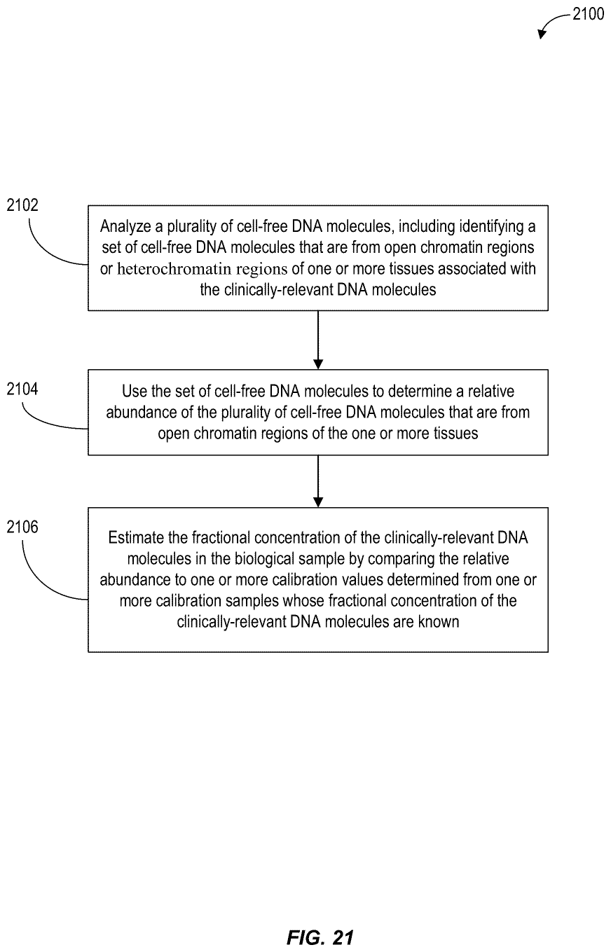

FIG. 21 shows a flowchart of a method 2100 for estimating a fractional concentration of clinically-relevant DNA molecules in a urine sample of a subject, according to some embodiments. The urine sample may include the clinically-relevant DNA and other DNA that are cell-free. In other examples, a biological sample may not include the clinically-relevant DNA, and the estimated fractional concentration may indicate zero or a low percentage of the clinically-relevant DNA.

The urine sample may include a mixture of cell-free DNA molecules from one or more tissue types, such as heart, lungs, and liver. For example, the urine sample may be obtained from a pregnant woman comprising maternal cell-free DNA molecules and fetal cell-free DNA molecules. The urine sample may comprise tumor specific cell-free DNA molecules as well as other tissue-specific cell-free DNA molecules. The clinically-relevant DNA molecules may comprise fetal DNA. In some embodiments, the clinically-relevant DNA include tumor DNA.

Aspects of method 2100 and any other methods described herein may be performed by a computer system.

In some instances, the urine sample is processed using a DNA stabilization agent prior to obtaining the cell-free DNA molecules. Different DNA stabilization agents can be used, such as EDTA and Collipee stabilization agent. EDTA can inhibit the cleavage activity of the DNASE1 family by chelating magnesium and calcium, which are the essential ions required for DNASE1 digestion. The stabilizers can potentially stabilize the urinary DNA from degradation.

The stabilizers could be but not limited to preservatives provided by Collipee company, diazolidinyl urea (DU), dimethylolurea, 2-bromo-2-nitropropane-1,3-diol, 5-hydroxymethoxymethyl-1-aza-3,7-dioxabicyclo (3.3.0) octane and 5-hydroxymethyl-1-aza-3,7-dioxabicyclo (3.3.0) octane and 5-hydroxypoly[methyleneoxy]methyl-1-aza-3,7-dioxabicyclo (3.3.0) octane, bicyclic oxazolidines (e.g. Nuosept95), DMDM hydantoin, imidazolidinyl urea (IDU), sodium hydroxymethylglycinate, hexamethylenetetramine chloroallyl chloride (Quaternium-15), biocides (such as Bioban, Preventol and Grotan), a water-soluble zinc salt, EDTA, other metal ion chelators such as N, N′-bis-(dithiocarboxy) piperazine (BDP), diethyldithiocarbamate (DDTC), iminodisuccinic acid (IDS), polyaspartic acid, S,S-Ethylenediamine-N,N′-disuccinic acid (EDDS), methylglycinediacetic acid (MGDA), etc.

At block 2102, a plurality of cell-free DNA molecules from the urine sample are analyzed. In some instances, the plurality of cell-free DNA molecules from the urine sample are analyzed to obtain sequence reads. As examples, the sequence reads can be obtained using sequencing or probe-based techniques, either of which may include enriching, e.g., via amplification or capture probes.

The sequencing may be performed in a variety of ways, e.g., using massively parallel sequencing or next-generation sequencing, using single molecule sequencing, and/or using double- or single-stranded DNA sequencing library preparation protocols. The skilled person will appreciate the variety of sequencing techniques that may be used. As part of the sequencing, it is possible that some of the sequence reads may correspond to cellular nucleic acids.

In some instances, analyzing the plurality of cell-free DNA molecules includes: (i) determining locations of the plurality of cell-free DNA molecules; and (ii) identifying, based on the locations, a set of cell-free DNA molecules that are from open chromatin regions of one or more tissues associated with the clinically-relevant DNA molecules. All OCRS or just a subset of OCRs can be used. For example, OCRs specific to tissues that produce (e.g., contribute to) transrenal DNA can be used. Any one or more of transrenal-specific OCRs can be used in embodiments of the present disclosure. Such regions may be referred to as transrenal open chromatin regions.

A location of a cfDNA molecule can be determined by aligning (mapping) one or more corresponding sequence reads to a reference genome. As another examples, a location can be defined based on a probe used, e.g., a identified by an emitted signal, such as a color for a fluorescent dye. In such a manner, it can be determined whether a cfDNA molecule is within a transrenal OCR.

The OCRs can be identified in various ways, as will be appreciated by the skilled person in light of the present disclosure. The open chromatin regions may include one or more DNase1 hypersensitive sites (DHS) defined using DNase-seq (Meuleman et al. Nature. 2020; 584:244-251). The open chromatin regions can include sites identified using DNase-seq, sites identified using Assay for Transposase-Accessible Chromatin using sequencing (ATAC-seq), transcriptional start sites (TSS), CCCTC-binding factor (CTCF) sites, enhancer sites, as well as other nuclease hypersensitive sites.

In some embodiments, analyzing the plurality of cell-free DNA molecules further includes identifying the set of cell-free DNA molecules that are from the open chromatin regions of one or more tissues and having sizes that are less than a specified size threshold. For example, as shown in the graph 2004 of FIG. 20, the relative abundance of the clinically-relevant DNA molecules can be calculated based on shorter DNA fragments (e.g., fragments less than 80 base pairs) that are from the open chromatin regions. The size threshold can filter for shorter DNA fragments in determining relative abundance of clinically-relevant DNA molecules, since the transrenal DNA (e.g., DNA molecules that pass through the GBM of the kidney) can be characterized by their shorter sizes. As described herein, an amount of the transrenal DNA can be used to identify clinically-relevant DNA molecules in the urine sample. As examples, the size threshold can be 40 base pairs, 50 base pairs, 60 base pairs, 70 base pairs, 80 base pairs, 90 base pairs, 100 base pairs, 110 base pairs, 120 base pairs, 130 base pairs, 140 base pairs, 150 base pairs, or 160 base pairs, which may be used in any embodiment using a size threshold as described herein.

A statistically significant number of cell-free DNA molecules can be analyzed so as to provide an accurate determination of the fractional concentration. In some embodiments, at least 1,000 cell-free DNA molecules are analyzed. In other embodiments, at least 5000 or 10,000 or 50,000 or 100,000 or 500,000 or 1,000,000 or 5,000,000 cell-free DNA molecules, or more, can be analyzed. In some instances, the set of cell-free DNA molecules include at least 1000, 2000, 3000, 4000, 5000, 10,000, 50,000, or 100,000 cell-free DNA molecules.

To identify the set of cell-free DNA molecules from the open chromatin regions, the urine sample can be enriched for DNA fragments from the OCRs (e.g., targeted sequencing), thereby creating an enriched sample. For example, a biological sample can be enriched for DNA fragments from open chromatin regions of the one or more tissues, such as CTCF sites, TSS sites, DNase1 hypersensitivity sites, or Pol II regions. The enriching can include using capture probes that bind to a portion of, or an entire genome, e.g., as defined by a reference genome. As another example, the enriching can use primers to amplify (e.g., via PCR, rolling circle amplification, or multiple displacement amplification (MDA) certain regions of the genome. In some instances, the enrichment of the includes using the set of cell-free DNA molecules that are from the open chromatin regions of the one or more tissues and having sizes that are less than the specified size threshold. In some embodiments, the urine sample is enriched for cell-free DNA molecules having multiple fragmentomic characteristics, including cell-free DNA molecules that: (i) are from the open chromatin regions of one or more tissues; (ii) have sizes that are less than a specified size threshold (e.g., 80 base pairs); and/or (iii) have one or more ending sequences that correspond to a sequence end signature (e.g., CC-ends).

At block 2104, the set of cell-free DNA molecules are used to determine a relative abundance of the plurality of cell-free DNA molecules that are from open chromatin regions of the one or more tissues. In some instances, the relative abundance may comprise a normalized end density. For example, the normalized end density can be calculated based on the count of fragment ends of the set of DNA molecules located within a window of various sizes around (e.g., 1-kb upstream and 1-kb downstream or other described herein) an OCR divided by the median or mean count across the loci flanking all OCRs An OCR may be defined in various ways, e.g., by CTCF sites, TSS sites, DNase1 hypersensitivity sites, Pol II region.

Accordingly, the end density can comprise a first amount of the set of cell-free DNA molecules that are from the open chromatin regions of the one or more tissues divided by a second amount of the plurality of cell-free DNA molecules from one or more other regions, e.g., regions that neighbor one or more of the OCRs, potentially all of the OCRs used. The second amount can be an amount of all of the plurality of cell-free DNA molecules, and thus the first amount can be a subset of the second amount.

In some embodiments, as previously shown in FIGS. 16-17, urine samples of healthy subjects can be expected to exhibit a particular amount of DNA molecules from the open chromatin regions, or a particular ratio (e.g., O/E ratio) between a first relative frequency (e.g., a percentage) of urinary DNA molecules from the OCRs (observed value) and a second relative frequency of reference sequences of a reference genome from open chromatin regions of the one or more tissues (expected value). The expected OCR-related DNA contribution can thus correspond to a theoretical percentage of OCR in a reference genome (e.g., a human reference genome). For example, the observed value (O) can include a percentage of the fragments aligned to OCR in all fragments and the expected value (E) as the theoretical percentage of OCR in the reference genome. In some instances, the expected value is determined based on a relative frequency of single-nucleotide variants of the reference genome that are from the open chromatin regions of the one or more tissues. In various examples, the relative abundance can be a ratio between the first relative frequency and the second relative frequency or a ratio of one of the frequencies and a sum of both values.

At block 2106, the fractional concentration of the clinically-relevant DNA molecules in the biological sample is estimated by comparing the relative abundance to one or more calibration values determined from one or more calibration samples whose fractional concentration of the clinically-relevant DNA molecules are known. As shown in FIGS. 19 and 20, the fetal and maternal DNA have different relative abundances. A sample having a mixture of both will have a relative abundance that depends on the proportion of fetal/maternal DNA in the sample. The fractional concentration for a calibration sample can be determined in other ways, e.g., using a locus on a Y chromosome for a male fetus or a fetal-specific marker (e.g., an allele inherited from the father or a fetal-specific epigenetic marker).

Calibration data points can include a relative abundance and a measured/known fraction of the clinically-relevant DNA. The comparison can involve comparing the relative abundance to a calibration curve (composed of the calibration data points), and thus the comparison can identify the point on the curve having the measured relative abundance for the test sample. For example, the relative abundance can be compared to the calibration curve by inputting the relative abundance to the calibration function that represents the calibration curve. The fractional concentration corresponding to the identified point can then be used to estimate the fractional concentration. For example, the relative abundance can be provided as an input to the calibration function (e.g., a linear or non-linear fit) to obtain an output of the fractional concentration.

Accordingly, comparing the relative abundance to the one or more calibration values can include comparing the relative abundance to a calibration curve that includes the one or more calibration values. And to obtain the calibration data points, some embodiments can, for each calibration sample of the one or more calibration samples, measure the fractional concentration of the clinically-relevant DNA molecules in the calibration sample and measure the relative abundance of cell-free DNA molecules from the calibration sample that are from the open chromatin regions of the one or more tissues. As described above, measuring the fractional concentration of the clinically-relevant DNA molecules can use a tissue-specific allele or a tissue-specific methylation pattern.

The fractional concentration is a quantitative value and may be a range of values. For example, the fractional concentration may identify that the quantitative value is greater than or less than a specified value. In other implementations, the fractional concentration can have an upper bound and a lower bound, which can correspond to a resolution for which the fractional concentration may be determined.

### B. Enriching Urine Samples Using End-Motif Characteristics

In some embodiments, transrenal urinary cell-free DNA fraction in urine samples can be determined using certain end motifs, e.g., as described in section IV and elsewhere herein. The end motifs may include, but are not limited to, end sequences with certain lengths (e.g., 1-mer, 2-mer, 3-mer, 4-mer, 5-mer, 6-mer, 7-mer, 8-mer, 9-mer). Further data is provided below.

### 1. Relation of C-End End Motifs to Fetal Fraction

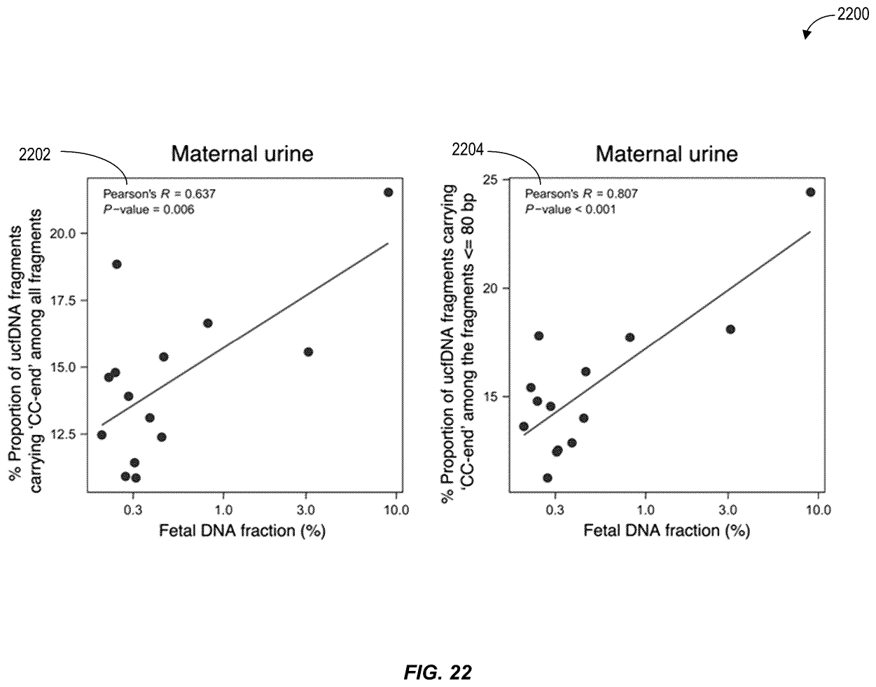

FIG. 22 shows a set of graphs 2200 that identify correlations between fetal DNA fraction and proportion of urinary cell-free DNA fragments carrying CC-ends. In FIG. 22, a graph 2202 shows correlations between fetal DNA fraction and proportion of all urinary cell-free DNA fragments carrying CC-ends in urine samples. A graph 2202 shows correlations between fetal DNA fraction and proportion of urinary cell-free DNA fragments carrying CC-ends that are equal to or less than 80 bp.

In the graph 2202, the fetal DNA fraction in maternal urine was significantly correlated with the proportional contribution of urinary cell-free DNA fragments carrying ‘CC-end’ among all fragments (Pearson's R=0.637; P-value=0.006). In the graph 2204, after selecting the fragments <=80 bp, we observed a further increase in terms of the correlation between fetal DNA fraction in maternal urine and the proportion of urinary cell-free DNA fragments carrying ‘CC-end’ (Pearson's R=0.807; P-value <0.001).

### 2. Example Enrichment Protocols

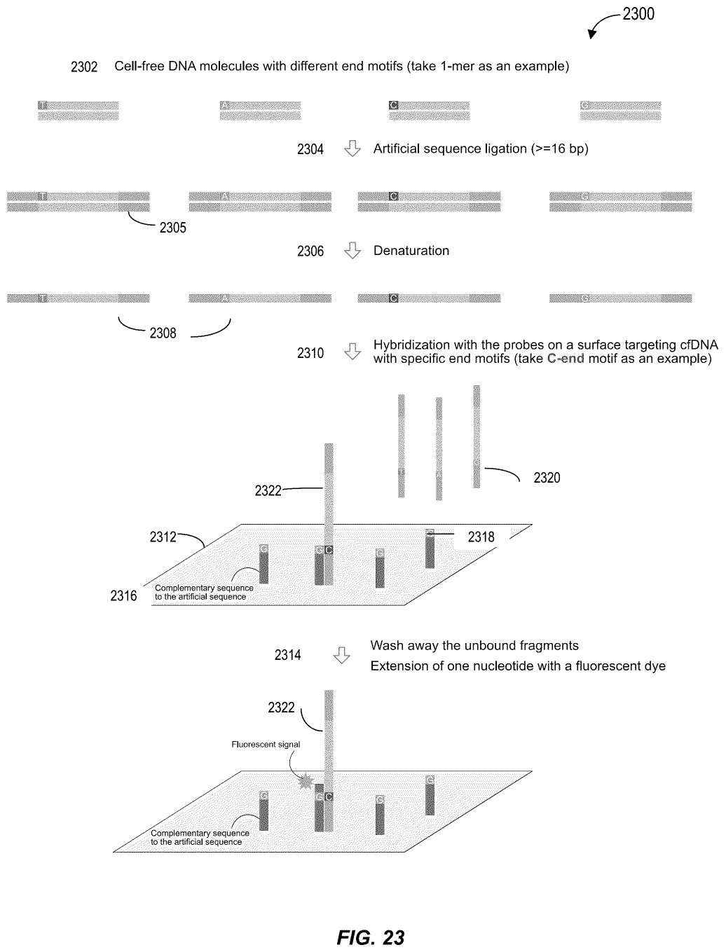

FIG. 23 illustrates a technique 2300 using probes for enriching a set of one or more end motifs. Technique 2300 can be used to enrich C-end motifs to enrich a urine sample for a clinically-relevant DNA, as described in FIGS. 9, 10, and 22.

As shown, cell-fee DNA molecules 2302 have different end motifs, e.g., 1-mer end motifs in this example.

In step 2304, the cfDNA fragments with different end motifs were ligated with a common sequence 2305, e.g., an artificial sequence. More than one sequence may be used, using one comment sequence can be more efficient. The length of the artificial sequences should be >=a specified length, such as 16 bp (or 17, 18, 19, 20, 21, 22, 23, 24, 25, or 26 bp) to ensure specificity for probe binding (4¹⁶>3×10⁹ [Human genome length]). The artificial sequence at the end of the DNA fragments can facilitate the probe recognition of the specific DNA end motifs.

In step 2306, the DNA molecules with common sequence 2305 are denatured to separate the two strands, resulting in single-stranded cfDNA 2308 having the different end motifs and common sequence 2305. Various denaturing protocols can be used, e.g., using temperature, as will be appreciated by the skilled person.

In step 2310, a surface 2312 (e.g., a chip surface) is fixed with many probe sequences 2316. Probe sequences 2316 have two components, containing a complementary sequence to the common sequence 2305 and the complementary motif sequence 2318 to the targeting end motif sequence (e.g., a “G” to target C-end motifs). Only fragments with targeted end motifs (e.g., having an end-C motif) could bind to the probes, leaving the fragments ending with other motifs unbound (i.e., unbound fragments 2320). The complementary motif sequence 2318 can be a set of different end motifs, e.g., if 2-mers or higher are used. For example, for 2-mers, four different probes can be used, for the four different 2-mers that end with C.

In step 2314, the unbound fragments 2320 are washed away. The remaining bound fragments 2322 can be detected or further analyzed in various ways. For example, only the probes having complementary motif sequence 2318 bound with fragments can be extended (e.g., by one nucleotide ligated with a fluorescent dye by DNA polymerase). In this manner, the fluorescent signal can be detected when there is a fragment carrying a targeted motif. As other examples for detection, a reaction can extend the bound cfDNA fragments by one nucleotide labelled with biotin. The biotin can be detected by streptavidin conjugated to fluorophores. As another option, a reaction can extend one nucleotide labelled with dinitrophenyl. The dinitrophenyl can be detected by anti-DNP antibodies that are labelled with fluorophores. In other implementations, the bound fragments can be sequenced in a separate process.

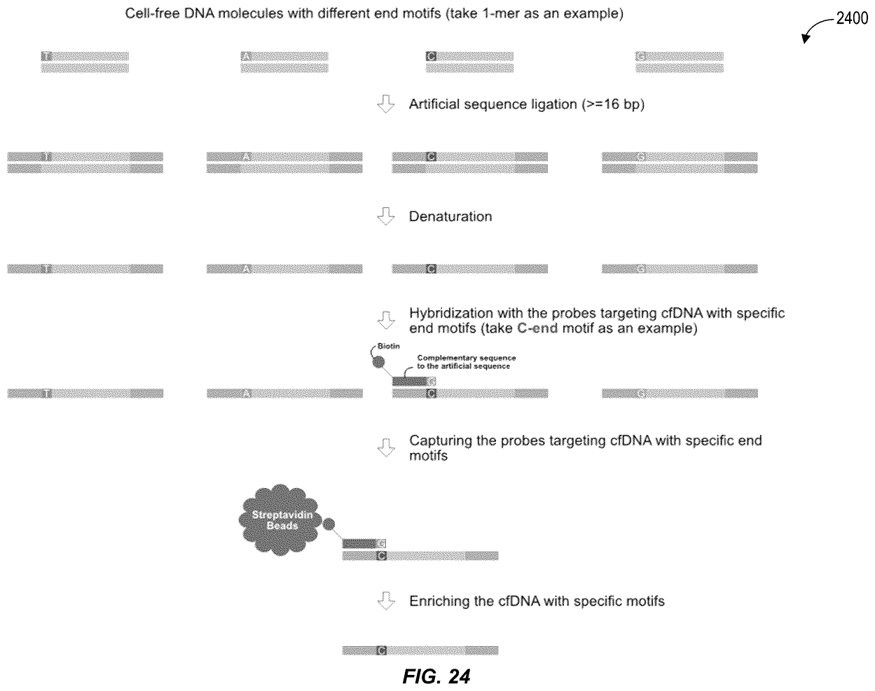

FIG. 24 illustrates another technique 2400 using probes and beads for enriching a set of one or more end motifs. Similar to FIG. 23, the cfDNA fragments with different end motifs can be ligated with artificial sequences. The artificial sequence at the end of the DNA fragments can facilitate the probe recognition of the specific DNA end motifs. The double-stranded DNA fragments can be denatured, becoming single-stranded DNA.

In the example shown, the probes targeting DNA fragments with specific end motifs have three components: biotin that can bind to the streptavidin beads, a complementary sequence to the common sequence, and a complementary motif sequence to the specific end motif sequence (e.g., a “G” to target C-end motifs). The probes are hybridized with the DNA fragments. Only fragments with specific end motifs could bind to the probes, leaving the fragments with other end motifs unbound.

Streptavidin beads can capture the probes because of the high affinity between the biotin and streptavidin. Only fragments with specific end motifs can be captured by the streptavidin beads. The unfound fragments are washed away. As a result, the cfDNA fragments with specific end motifs can be captured by such a design.

The fragments bound to the complementary motif sequence using technique 2400 can be detected or further analyzed in a same manner as technique 2300.

Instead of washing away unbound fragments in order to enrich the target end motif(s), the target end motif(s) can be amplified. For example, primers that include the common sequence and the target end motif can be added to a reaction, along with nucleotides, and an amplification process (e.g., PCR or rolling circle) can be performed.

### 3. Method

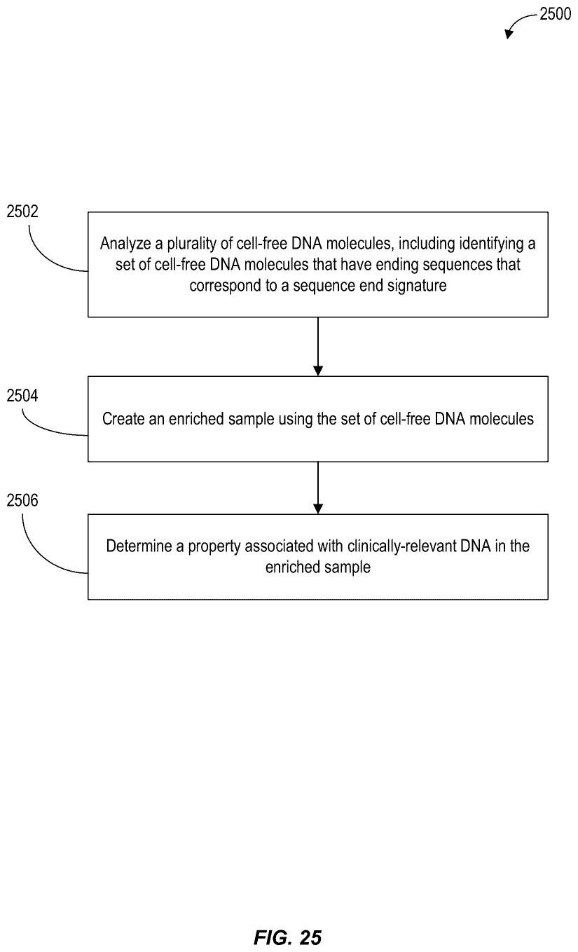

FIG. 25 shows a flowchart of a method 2500 for enriching a urine sample for clinically-relevant DNA based on end-motif characteristics of urinary cell-free DNA, according to some embodiments. Aspects of method 2500 and other methods described herein may be performed in a similar manner as method 2100, e.g., the sample preparation and analysis of DNA molecules. The urine sample may include the clinically-relevant DNA and other DNA that are cell-free. In other examples, a biological sample may not include the clinically-relevant DNA, and the estimated fractional concentration may indicate zero or a low percentage of the clinically-relevant DNA. Aspects of method 2500 and any other methods described herein may be performed by a computer system.

At block 2502, a plurality of cell-free DNA molecules from the urine sample are analyzed. Aspects of block 2502 may be performed in a similar manner as block 2102 of method 2100. For example, the plurality of cell-free DNA molecules from the urine sample can be analyzed to obtain sequence reads. The sequence reads can include ending sequences corresponding to ends of the plurality of cell-free DNA fragments. As examples, the sequence reads can be obtained using sequencing or probe-based techniques, either of which may include enriching, e.g., via amplification or capture probes.

The sequencing may be performed in a variety of ways, e.g., using massively parallel sequencing or next-generation sequencing, using single molecule sequencing, and/or using double- or single-stranded DNA sequencing library preparation protocols. The skilled person will appreciate the variety of sequencing techniques that may be used. As part of the sequencing, it is possible that some of the sequence reads may correspond to cellular nucleic acids.

In some embodiments, analyzing the plurality of cell-free DNA molecules further includes identifying the set of cell-free DNA molecules that are in a set of one or more sequence motifs that include a C-end nucleotide. The sequence end signature may be part of a K-mer end motif, e.g., a 2-mer, 3-mer, 4-mer, etc. For example, the set of cell-free DNA molecules are further identified based on having CC-ends. Further, the ending sequences can be required to be on both ends of a DNA fragment, or a particular pair of different end motifs can be used to select a particular set of DNA fragments.

When sequencing is performed, identifying the set of the plurality of cell-free DNA molecules can include identifying sequence reads having the ending sequences that are in the set of one or more sequence motifs. Thus, the enriched sample can correspond to the sequence reads having the ending sequences that are in the set of one or more sequence motifs. As an alternative than sequencing, to identify the DNA molecules having the one or more ending sequences, one or more probe molecules can be attached to a surface and detect the sequence motifs in the ending sequences by hybridization.

In some embodiments, the set of cell-free DNA molecules are further identified based on their respective sizes (e.g., fragments less than 80 base pairs). As shown in FIG. 22, the size threshold can filter for shorter DNA fragments, since the transrenal DNA (e.g., DNA molecules that pass through the GBM of the kidney) can be characterized by their shorter sizes. As described herein, an amount of the transrenal DNA can be used to identify clinically-relevant DNA molecules in the urine sample. The specified size threshold can include 40 base pairs, 50 base pairs, 60 base pairs, 70 base pairs, 80 base pairs, 90 base pairs, 100 base pairs, 110 base pairs, 120 base pairs, 130 base pairs, 140 base pairs, 150 base pairs, or 160 base pairs.

A statistically significant number of cell-free DNA molecules can be analyzed, as described herein.

At block 2504, an enriched sample can be created by using the set of cell-free DNA molecules that are in the set of one or more sequence motifs. The enriched sample thus includes a higher concentration of clinically-relevant DNA compared to the urine sample. The enriched sample can be an in silico sample, in that the measurements of only certain cfDNA molecules are used. In other examples, the enriched sample may be a physical sample.

The enriching can include using capture probes that bind to the set of one or more sequence motifs. For example, identifying the set of cell-free DNA molecules or creating the enriched sample can include subjecting the plurality of cell-free DNA molecules to one or more probe molecules that detect the set of one or more sequence motifs in the ending sequences of the plurality of cell-free DNA molecules. Use of such probe molecules can obtain the set of cell-free DNA molecules. As described for FIGS. 23 and 24, some embodiments can attach a common sequence to the plurality of cell-free DNA molecules. The one or more probe molecules can then include a complementary sequence to the common sequence.

In some instances, creating the enriched sample includes capturing the set of cell-free DNA molecules using probe molecules and discarding other cell-free DNA molecules of the plurality of cell-free DNA molecules. In other instances, creating the enriched sample can include amplifying the set of cell-free DNA molecules using the one or more probe molecules.

The capture probes can also that bind to (target) a portion of, or an entire genome, e.g., as defined by a reference genome. As another example, the enriching can use primers to amplify (e.g., via PCR, rolling circle amplification, or multiple displacement amplification (MDA) certain regions of the genome.

In some embodiments, the urine sample is enriched for cell-free DNA molecules having multiple fragmentomic characteristics, including cell-free DNA molecules that: (i) are from the open chromatin regions of one or more tissues; (ii) have sizes that are less than a specified size threshold (e.g., 80 base pairs); and/or (iii) have one or more ending sequences that correspond to a sequence end signature (e.g., C-ends or CC-ends of a K-mer end motif).

At block 2506, a property associated with the clinically-relevant DNA in the enriched urine sample is determined. As examples, the property of the clinically-relevant DNA in the urine sample can be (1) a fractional concentration of the clinically-relevant DNA or (2) a level of pathology of a subject from whom the biological sample was obtained, e.g., where the level of pathology is associated with the clinically-relevant DNA. The skilled person will appreciate the various properties that can be determined, e.g., fetal inheritance of haplotype, detecting mutations, copy number aberrations (e.g., aneuploidy), methylation properties, various base modifications, genomic interactions, protein-binding status, fragmentomic features, and the like using the set of cell-free DNA molecules having ending sequences that are in a set of one or more sequence motifs that include a C-end nucleotide, as described variously in US Publication Nos. 2009/0087847, 2009/0029377, 2011/0276277, 2011/0105353, 2013/0040824, 2014/0100121, 2014/0080715, and 2020/0199656.

### C. Enriching Urine Samples Using OCRs and Other Features

The above fragmentomic features (e.g., end motifs, size, enrichment of open chromatin regions) can be combined to estimate transrenal DNA contributions. For example, fetal DNA molecules can be enriched with CC-ends. Based on this correlation, contribution of transrenal DNA can be estimated based on proportions of urinary cell-free DNA that have CC-ends in urine samples. If the proportion of urinary cell-free DNA having CC-ends and sizes (e.g., fragments shorter than 80 bp) are used together, estimating transrenal DNA contribution in urine samples can become more accurate. In effect, the accuracy of estimating fetal DNA fraction can improve as well.

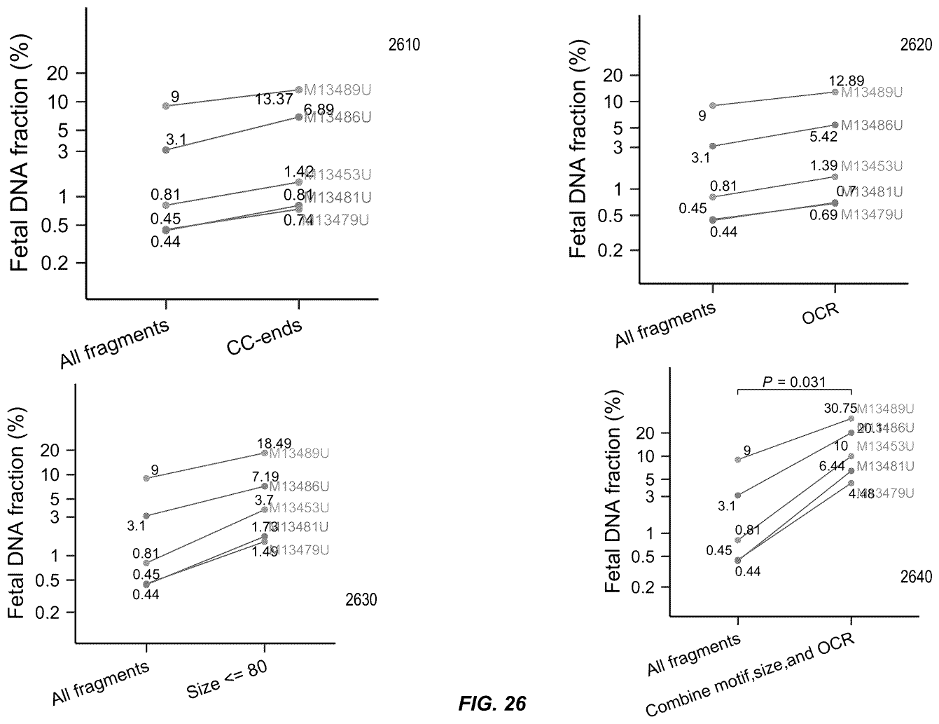

FIG. 26 shows a set of graphs that identify enrichment of fetal DNA using urinary cell-free DNA having various fragmentomic properties, according to some embodiments. As shown in FIG. 26, the DNA molecules filtered for CC-ends (graph 2610), OCRs (graph 2620), and sizes being less than 80 base pair (graph 2630) can result in a substantial increase of enrichment of fetal DNA in urine samples. The enrichment is further pronounced using a combination (graph 2640) compared to individually using the above fragmentomic features.

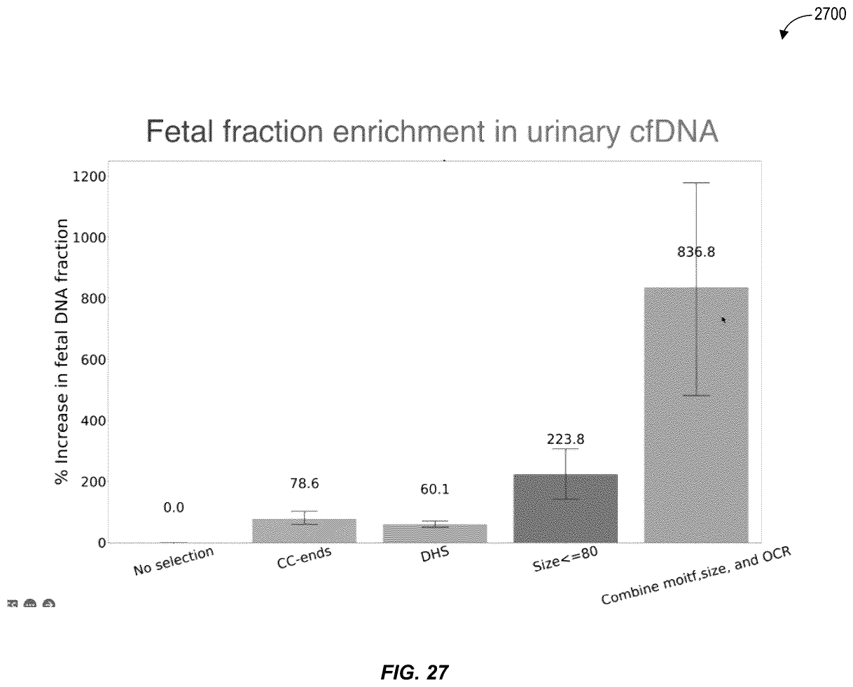

FIG. 27 shows a bar graph 2700 that identify enrichment of transrenal urinary cell-free DNA using selective analysis of fragments with different fragmentomic features. The percentage increase in fetal DNA fraction in urinary cell-free DNA with no selection 2702, with CC-end based selective analysis 2704, OCR-based selective analysis 2706, size-based selection (<=80 bp) 2708, and a combination of features corresponding to end motif, OCR, and size 2710. To show the increases in fetal DNA fraction, we calculated the means of the increase in fractional concentration of fetal DNA after different criteria selection.

As shown in FIG. 27, if fragments were filtered for having CC-ends, within OCR regions, or having sizes of equal or less than 80 bp, the fetal DNA fraction in a given urine sample increased by 78.6%, 60.1%, and 223.8%, respectively. In other words, filtering DNA molecules using CC-end and OCR region criteria caused the fractional concentration of fetal DNA to increase around one fold. If size (fragments equal to or less than 80 bp) criteria was used as the filter the DNA molecules, the fractional concentration of fetal DNA increased around two fold. If one combined these three fragmentomic features together, the fetal DNA fraction in urine could be further increased by 836.8% (more than eight fold). The data in FIG. 27 thus indicates that one can enrich the target transrenal urinary cell-free DNA on the basis of selective analysis of cell-free DNA molecules according to different combinations of fragmentomic features.

In addition, a combination of two or more of these fragmentomic features can also be used in estimating contribution of fetal DNA in a urine sample. For example, method 2100 can further use a statistical size of a size distribution, as described in U.S. Pat. No. 9,892,230. As another example, additionally or alternatively to using OCRs, a set of one or more sequence motifs that include a C-end nucleotide can be used. Each of these different features can be used together, e.g., in a two-dimensional or three-dimensional calibration curve.

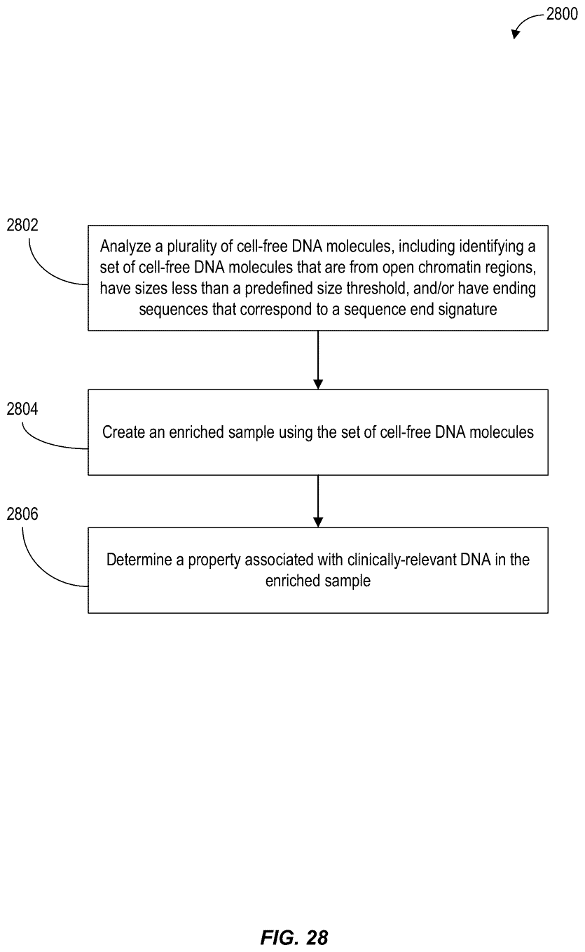

FIG. 28 shows a flowchart of a method 2800 for enriching a urine sample for clinically-relevant DNA based on end motifs, open chromatin regions according to some embodiments. Aspects of method 2800 and other methods described herein may be performed in a similar manner as method 2100 and/or method 2500, e.g., the sample preparation and analysis of DNA molecules. The urine sample may include the clinically-relevant DNA and other DNA that are cell-free. In other examples, a biological sample may not include the clinically-relevant DNA, and the estimated fractional concentration may indicate zero or a low percentage of the clinically-relevant DNA. Aspects of method 2800 and any other methods described herein may be performed by a computer system.

At block 2802, a plurality of cell-free DNA molecules from the urine sample are analyzed. Aspects of block 2802 may be performed in a similar manner as block 2102 or block 2502. For example, the plurality of cell-free DNA molecules from the urine sample are analyzed to obtain sequence reads. The sequence reads can include ending sequences corresponding to ends of the plurality of cell-free DNA fragments. As examples, the sequence reads can be obtained using sequencing or probe-based techniques, either of which may include enriching, e.g., via amplification or capture probes, as is described for method 2500..

In some embodiments, analyzing the plurality of cell-free DNA molecules further includes identifying the set of cell-free DNA molecules that: (i) are from the open chromatin regions of one or more tissues; (ii) have sizes that are less than a specified size threshold (e.g., 80 base pairs); and/or (iii) have one or more ending sequences that correspond to a sequence end signature (e.g., C-ends or CC-ends of a K-mer end motif). The open chromatin regions can be identified in a similar manner as described herein.

As shown in FIGS. 26 and 27, the set of cell-free DNA molecules can be identified based on their respective sizes (e.g., fragments less than 80 base pairs). The size threshold can filter for shorter DNA fragments, since the transrenal DNA (e.g., DNA molecules that pass through the GBM of the kidney) can be characterized by their shorter sizes. As described herein, an amount of the transrenal DNA can be used to identify clinically-relevant DNA molecules in the urine sample. The specified size threshold can include 40 base pairs, 50 base pairs, 60 base pairs, 70 base pairs, 80 base pairs, 90 base pairs, 100 base pairs, 110 base pairs, 120 base pairs, 130 base pairs, 140 base pairs, 150 base pairs, or 160 base pairs.

In some embodiments, various methods (e.g., gel electrophoresis) can be used to determine the sizes of the plurality of cell-free DNA molecules. For example, sizes of the plurality of cell-free DNA molecules can be measured using gel electrophoresis, filtration, size-selective precipitation, or hybridization. Additionally or alternatively, sizes of the plurality of cell-free DNA fragments can be measured using the sequence reads. For example, the sequence reads can be obtained from a sequencing (e.g., massively-parallel sequencing, single-molecule real-time sequencing, nanopore sequencing) of the plurality of cell-free DNA molecules from the biological sample. To measure sizes of cell-free DNA molecules, a number of nucleotides can be counted for each sequence read. The skilled person will appreciate the variety of sequencing techniques that may be used. As part of the sequencing, it is possible that some of the sequence reads may correspond to cellular nucleic acids. In some instances, the urine sample can be enriched for DNA fragments having sizes that are less than a predefined size threshold (e.g., 80 bps).

The set of cell-free DNA molecules can be further identified based on having one or more ending sequences that correspond to a sequence end signature. The sequence end signature may be part of an end motif, e.g., a 2-mer, 3-mer, etc. For example, the set of cell-free DNA molecules are further identified based on having CC-ends. Further, the ending sequences can be required to be on both ends of a DNA fragment, or a particular pair of different end motifs can be used to select a particular set of DNA fragments. Other than sequencing, to identify the DNA molecules having the one or more ending sequences, one or more probe molecules can be attached to a surface or a bead and detect the sequence motifs in the ending sequences by hybridization.

A statistically significant number of cell-free DNA molecules can be analyzed as described herein.

At block 2804, an enriched sample can be created by using the set of cell-free DNA molecules that: (i) are from the open chromatin regions of one or more tissues; (ii) have sizes that are less than a specified size threshold (e.g., 80 base pairs); and/or (iii) have one or more ending sequences that correspond to a sequence end signature (e.g., CC-ends). Aspects of block 2804 may be performed in a similar manner as block 2504. The enriched sample thus includes a higher concentration of clinically-relevant DNA compared to the urine sample. For example, a biological sample can be enriched for DNA fragments from open chromatin regions of the one or more tissues, such as CTCF sites, TSS sites, DNase1 hypersensitivity sites, or Pol II regions. The enriching can include using capture probes that bind to a portion of, or an entire genome, e.g., as defined by a reference genome. As another example, the enriching can use primers to amplify (e.g., via PCR, rolling circle amplification, or multiple displacement amplification (MDA) certain regions of the genome.

In some instances, creating the enriched sample includes capturing the set of cell-free DNA molecules using probe molecules and discarding other cell-free DNA molecules of the plurality of cell-free DNA molecules. The enriched sample can be an in silico sample.

At block 2806, a property associated with the clinically-relevant DNA in the enriched urine sample is determined. Aspects of block 2806 may be performed in a similar manner as block 2506. As examples, the property of the clinically-relevant DNA in the urine sample can be (1) a fractional concentration of the clinically-relevant DNA or (2) a level of pathology of a subject from whom the biological sample was obtained, e.g., where the level of pathology is associated with the clinically-relevant DNA. The skilled person will appreciate the various properties that can be determined, e.g., as described above for method 2500.

### VII. CLASSIFICATION OF ABNORMALITIES USING URINARY CFDNA

In some embodiments, cancers can be detected and monitored by using transrenal urinary cell-free DNA molecules. For example, renal cell carcinoma (RCC) is a disease in which malignant cells are found in the lining of tubules in the kidney. If the kidney function is affected, the fractional concentration of transrenal urinary cell-free DNA could be altered. In effect, patients with kidney cancer would exhibit aberrations in the fractional concentration of transrenal urinary cell-free DNA when compared with subjects without kidney cancer. Other kidney abnormalities (besides RCC) can also effect the transrenal urinary cell-free DNA in a urine sample, e.g., based on size or region, such as OCRs. Other examples include proteinuria and preeclampsia.

### A. Classification Based on OCRs

Urine samples of healthy subjects can be expected to exhibit a particular amount of DNA molecules from the open chromatin regions of one or more tissues, or a particular ratio between an observed frequency of urinary DNA molecules from the open chromatin regions and an expected frequency of reference sequences of a reference genome that are from open chromatin regions of one or more tissues. But, if permeability of GBM is disturbed in some subjects (e.g., subjects with nephrotic syndrome, glomerulonephritis), the above ratio may increase or decrease. If such changes relative to the normal amount exceeds a predefined threshold, the subjects can be determined to have diseases or other abnormal conditions that affect permeability of the kidney.

An amount of DNA molecules from open chromatin regions of one or more tissues can be measured for control subjects. Then, a substantial deviation from the above measured amount of DNA molecules can be used to determine whether a given subject has an abnormality for the kidney. For example, blood samples can include cell-free DNA molecules originating from different organs (e.g., heart, lungs, liver). An amount of cell-free DNA molecules that correspond to open chromatin regions of the liver (for example) can be determined for urine samples (e.g., using targeted sequencing of the OCRs). If there is a statistically significant difference between the determined amount of cell-free DNA molecules and a calibration amount of cell-free DNA molecules corresponding to open chromatin regions of healthy subjects, a classification of kidney abnormality can be determined. More than one tissue-specific region can be used. Collectively the measurement can be for all OCRs or ones that are specific to one or more tissues contributing transrenal DNA or specific to one or more cell types, as described in previous sections.

### 1. Renal Cell Carcinoma

For illustration purposes, we analyzed the O/E ratio of urinary cell-free DNA from 15 control subjects and O/E ratio of 16 patients with renal cell carcinoma (RCC). To calculate the O/E ratio of all urinary cell-free DNA fragments, observed OCR-related DNA contribution can be determined as the percentage of the fragments aligned to OCR in all fragments. Expected OCR-related DNA contribution can be defined as the theoretical percentage of OCR in the human genome.

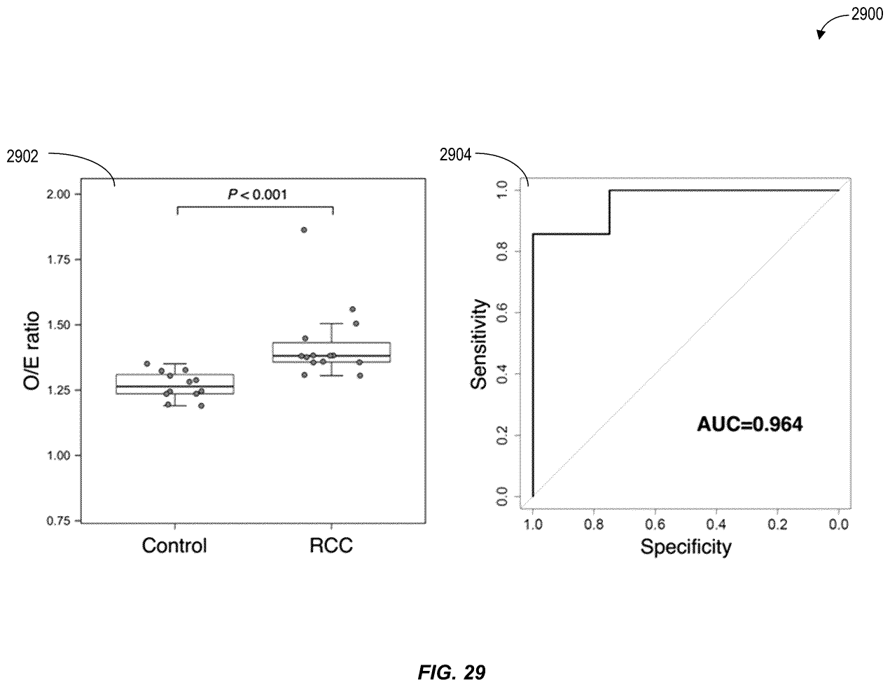

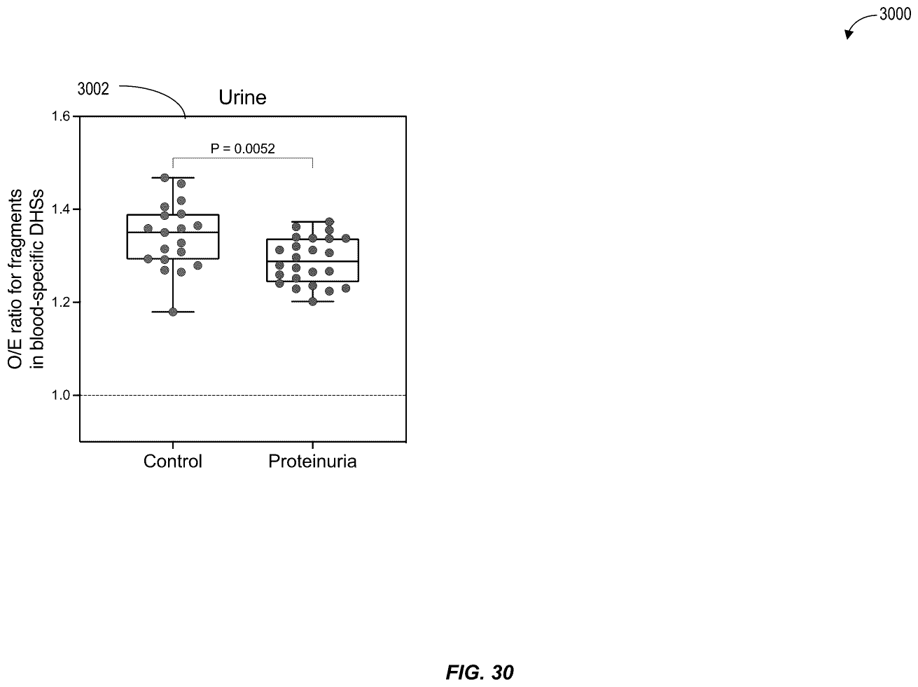

FIG. 29 shows a set of graphs 2900 that identify O/E ratio analysis in patients with RCC. Boxplot 2902 shows a boxplot of the O/E ratio between control subjects and patients with RCC. As shown in the boxplot 2902, the O/E ratios in RCC patients (median: 1.378; range: 1.174-1.863) were significantly higher than that in control subjects (median: 1.288; range: 1.190-1.571) (Mann-Whitney, P-value <0.001). In addition, we further performed the receiver operating characteristic (ROC) analysis on these samples. ROC 2904 shows an ROC of performance levels for differentiating patients with RCC from control subjects. The area under the curve (AUC) of the ROC 2904 was 0.964 in differentiating RCC patients from control subjects.

### 2. Proteinuria

Proteinuria, also called albuminuria, is elevated protein in the urine and can be considered a kidney abnormality. Because the kidney function is not functioning well, it will allow more protein to go into the urine and thus is a type of kidney abnormality.

Since the patients with proteinuria have excess proteins in their urine, we hypothesized that we could identify proteinuria patients from healthy controls using the fragmentomic features of urinary cfDNA. We used abundance for OCRs. Any transrenal-specific OCRs can be used. As with other embodiments of this disclosure, only OCR would be used for any given use case (e.g., classification of kidney abnormality, estimate of fractional concentration, or enrichment), although two separate determinations could be performed and then combined.

Since there is no placental DNA in the urine of healthy control and subjects with proteinuria, we use blood-associated regions to represent the transrenal-related genomic locations/regions.

FIG. 30 shows fragmentomic analysis of transrenal DNA in the patients with proteinuria. Boxplot 3002 shows an O/E ratio for urinary cfDNA fragments in OCR (blood-specific DHSs) in healthy controls and patients with proteinuria.

In the O/E ratio analysis, patients with proteinuria have significantly lower O/E ratios for fragments in OCR (blood-specific DHSs) (Mann-Whitney U test, P-value=0.0052). These results demonstrated decreased proportions of fragments from OCR in patients with proteinuria.

An ROC analysis is provided later.

### 3. Preeclampsia

We hypothesized that we could identify pregnant women with preeclampsia using the fragmentomic features of urinary cfDNA. Pregnant women with preeclampsia were usually diagnosed with elevated protein levels in the urine, indicating impaired GBM function in the kidney. We speculated that if large-size plasma molecules such as proteins could go through the GBM and enter the urine, then large-size DNA molecules from plasma (e.g., long DNA molecules or DNA molecules bound with histones) could also enter the urine.

We used DHSs to represent OCRs. Other ways to identify OCRs is described elsewhere in this disclosure.

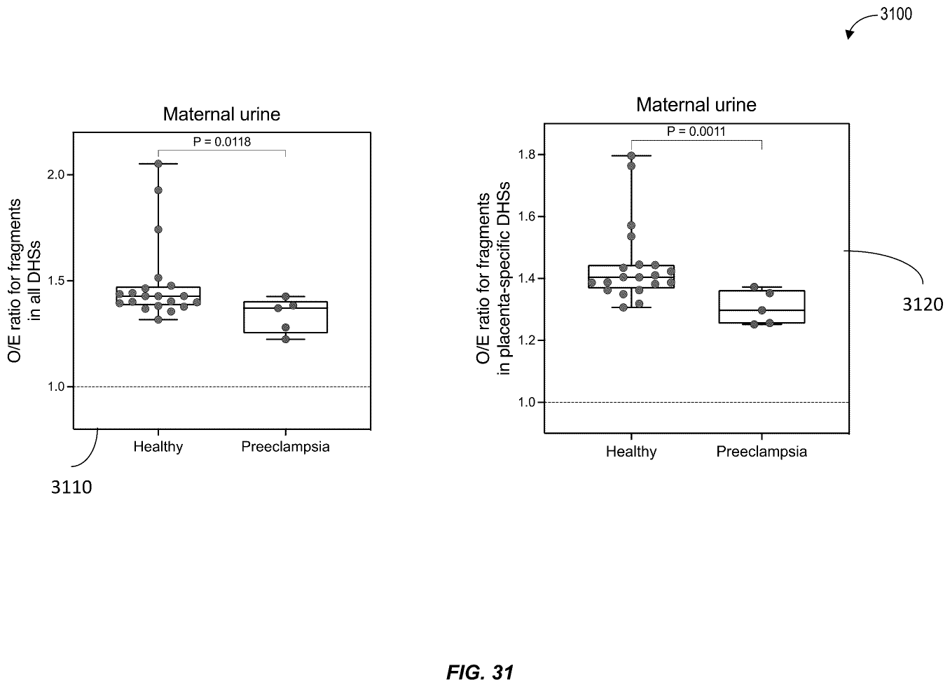

FIG. 31 shows fragmentomic analysis of transrenal DNA in the pregnant women with preeclampsia. Boxplot 3110 shows O/E ratio for urinary cfDNA fragments in OCR (all DHSs) in healthy pregnancy women and women with preeclampsia. Boxplot 3120 shows O/E ratio for urinary cfDNA fragments in OCR (placenta-specific DHSs) in healthy pregnancy women and women with preeclampsia.

The performance in differentiating the healthy pregnant women and the women with preeclampsia using the O/E ratio for fragments was better in placenta-specific DHSs (boxplot 3120) than in all DHSs (boxplot 3110) (Mann-Whitney U test, P-value: 0.0011 vs 0.0118). Thus, we used tissue-specific regions for O/E ratio analysis in the urine of subjects with preeclampsia and proteinuria. Compared with healthy pregnant women, pregnant women with preeclampsia have significantly lower O/E ratios for fragments in OCR (placenta-specific DHSs) (Mann-Whitney U test, P-value=0.0011). These data indicated decreased proportions of fragments from OCR in patients with preeclampsia.

When the kidney abnormality is preeclampsia additional factors can be used. For example, a determination of whether hypertension is present can also be used. For instance, a blood pressure can be compared to a threshold to determine whether the subject has hypertension. Another factor can be whether protein is present in the urine, e.g., proteinuria.

### 4. Method

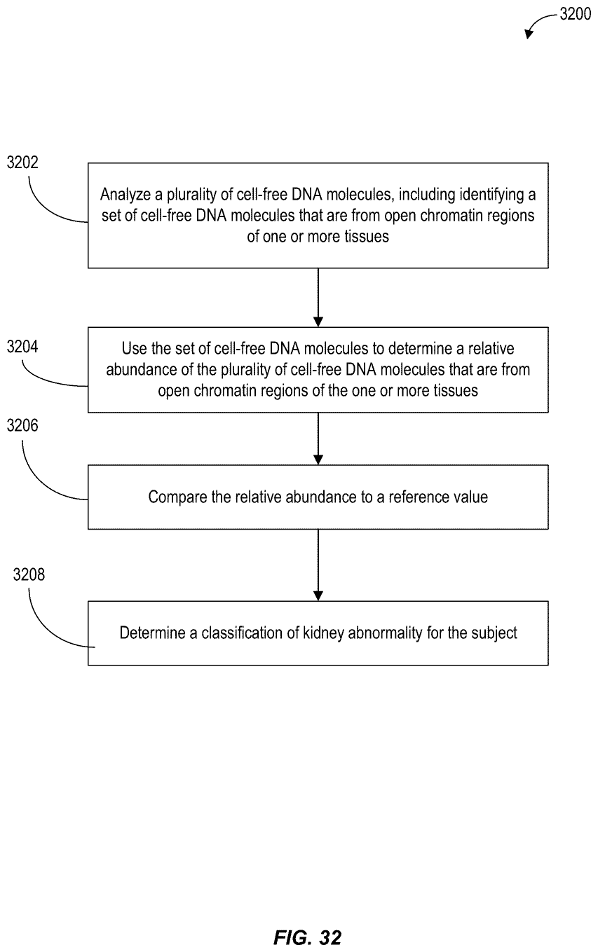

FIG. 32 shows a flowchart of a method 3200 for determining a classification of kidney abnormality based on urinary cell-free DNA from open chromatin regions, according to some embodiments. Aspects of method 3200 and other methods described herein may be performed in a similar manner as methods above, e.g., the sample preparation and analysis of DNA molecules. The urine sample may include a mixture of cell-free DNA molecules from one or more tissue types, such as heart, lungs, and liver. The urine sample may comprise tumor specific cell-free DNA molecules as well as other tissue-specific cell-free DNA molecules. For example, the urine sample can include cell-free DNA molecules specific to renal cell carcinoma (RCC). Aspects of method 3200 and any other methods described herein may be performed by a computer system.

At block 3202, a plurality of cell-free DNA molecules from the urine sample are analyzed. Aspects of block 3202 may be performed in a similar manner as similar blocks of other methods, such as block 2102 of method 2100, as can be done for other methods herein. For example, the plurality of cell-free DNA molecules from the urine sample are analyzed to obtain sequence reads. As examples, the sequence reads can be obtained using sequencing or probe-based techniques, either of which may include enriching, e.g., via amplification or capture probes.

In some instances, analyzing the plurality of cell-free DNA molecules includes: (i) determining locations of the plurality of cell-free DNA molecules; and (ii) identifying, based on the locations, a set of cell-free DNA molecules that are from open chromatin regions of one or more tissues associated with the clinically-relevant DNA molecules. The one or more tissues can include at least one of heart, lungs, or liver. The open chromatin regions can include one or more DNase1 hypersensitive sites (DHS) defined using DNase-seq (Meuleman et al. Nature. 2020; 584:244-251). The open chromatin regions can be identified as described herein.

A statistically significant number of cell-free DNA molecules can be analyzed as described herein.

To identify the set of cell-free DNA molecules from the open chromatin regions, the urine sample can be enriched for DNA fragments from the open chromatin regions (e.g., targeted sequencing), thereby creating an enriched sample. For example, a biological sample can be enriched for DNA fragments from open chromatin regions of the one or more tissues, such as CTCF sites, TSS sites, DNase1 hypersensitivity sites, or Pol II regions. The enriching can include using capture probes that bind to a portion of, or an entire genome, e.g., as defined by a reference genome. As another example, the enriching can use primers to amplify (e.g., via PCR, rolling circle amplification, or multiple displacement amplification (MDA) certain regions of the genome. In some instances, the enrichment of the includes using the set of cell-free DNA molecules that are from the open chromatin regions of the one or more tissues and having sizes that are less than the specified size threshold.

At block 3204, a relative abundance of the plurality of cell-free DNA molecules that are from open chromatin regions of the one or more tissues. Aspects of block 3204 may be performed in a similar manner as similar blocks of other methods, such as block 2104 of method 2100, as can be done for other methods herein. In some instances, the relative abundance may comprise a normalized end density. For example, the normalized end density can be calculated based on the count of fragment ends of the set of DNA molecules located within the 1-kb upstream and 1-kb downstream of an OCR (e.g., CTCF sites, TSS sites, DNase1 hypersensitivity sites, Pol II region) divided by the median count across the loci flanking all OCRs.

In some embodiments, as previously shown in FIGS. 29-31, urine samples of healthy subjects can be expected to exhibit a particular amount of DNA molecules from the open chromatin regions, or a particular ratio (e.g., O/E ratio) between a first relative frequency (e.g., a percentage) of urinary DNA molecules from the OCRs (observed value) and a second relative frequency of reference sequences of a reference genome from open chromatin regions of the one or more tissues (expected value). The expected OCR-related DNA contribution can thus correspond to a theoretical percentage of OCR in a reference genome (e.g., a human reference genome). For example, the observed value (O) can include a percentage of the fragments aligned to OCR in all fragments and the expected value (E) as the theoretical percentage of OCR in the reference genome. In some instances, the expected value is determined based on a relative frequency of single-nucleotide variants of the reference genome that are from the open chromatin regions of the one or more tissues. But, if permeability of GBM is disturbed in some subjects, the above ratio may increase or decrease. If such changes relative to the normal amount exceeds a predefined threshold, the subjects can be determined to have diseases or other abnormal conditions that affect permeability of the kidney (e.g., nephrotic syndrome, glomerulonephritis).

At block 3206, the relative abundance value is compared to a reference value. The reference value can correspond to another relative abundance determined based on cell-free DNA molecules that are from open chromatin regions of one or more reference samples, in which the one or more reference samples are associated with known classifications of the kidney abnormality. For example, the reference value can correspond to a relative abundance determined from healthy subjects. In some instances, the reference value is a calibration value or determined from calibration values of calibration samples. As with other reference values, the specific value selected can depend on a tradeoff of specificity and sensitivity. In some embodiments, the comparison can be performed using a machine learning model.

At block 3208, a classification of the subject having a kidney abnormality is determined based on the comparison. In some embodiments, comparing the relative abundance to the reference value includes: (1) determining whether the relative abundance differs from the reference value by at least a threshold amount or the difference is less than the threshold amount; (2) determining whether the relative abundance is less than the reference value by at least a threshold amount; or (3) determining whether the relative abundance is greater than the reference value by at least a threshold amount. As examples, the kidney abnormality can include renal cell carcinoma RCC, nephrotic syndrome, glomerulonephritis, Fabry disease, cystinosis, IgA nephropathy, IgM nephropathy, lupus nephritis, atypical hemolytic uremic syndrome (aHUS), polycystic kidney disease (PKD), Alport's syndrome, interstitial nephritis, proteinuria, chronic kidney disease, acute kidney injury, proteinuria, preeclampsia, etc. In some instances, the classification of the subject having the kidney abnormality includes an increased level of permeability associated with a glomerular basement membrane of the kidney.

The classification of the kidney abnormality can be determined using machine learning trained using a training dataset. The training dataset can include training samples. The training samples can be associated with known classifications of the kidney abnormality. In another example, the comparison to the reference value can be performed using a machine learning model. The machine-learning model can be applied to the relative abundance to generate the classification of the kidney abnormality. The machine learning models can include, but not limited to, convolutional neural network (CNN), linear regression, logistic regression, deep recurrent neural network (e.g., fully-connected recurrent neural network (RNN), Gated Recurrent Unit (GRU), long short-term memory, (LSTM)), transformer-based methods (e.g. XLNet, BERT, XLM, RoBERTa), Bayes's classifier, hidden Markov model (HMM), linear discriminant analysis (LDA), k-means clustering, density-based spatial clustering of applications with noise (DBSCAN), random forest algorithm, adaptive boosting (AdaBoost), eXtreme Gradient Boosting (XGBoost), support vector machine (SVM), or a composite model comprising one or more models proposed above.

### B. Classification Based on Sizes

In addition or alternatively from a classification using OCRs, a classification can be made using size of the urinary cfDNA. The classification of the kidney abnormality can be performed in a similar manner, but instead using a statistical of a size distribution of the sizes of cfDNA in the urine sample.

### 1. Proteinuria and Preeclampsia

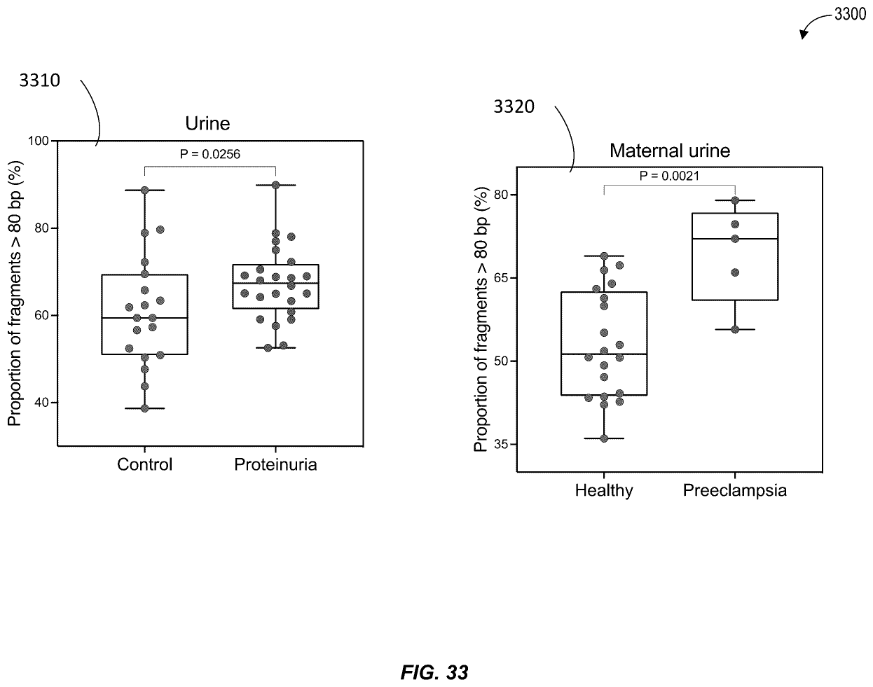

FIG. 33 is a fragmentomic analysis 3300 of transrenal DNA in the patients with proteinuria and separately with preeclampsia.

Boxplot 3310 shows the proportion of urinary cfDNA >80 bp in healthy controls and patients with proteinuria. We observed higher proportions of long urinary cfDNA fragments (i.e., >80 bp) (Mann-Whitney U test, P-value=0.0256) in patients with proteinuria than in healthy controls.

Boxplot 3320 shows the proportion of urinary cfDNA >80 bp in healthy pregnant women and women with preeclampsia. We observed higher proportions of long urinary cfDNA fragments (i.e., >80 bp) (Mann-Whitney U test, P-value=0.0021) in pregnant women with preeclampsia than in healthy pregnant women.

### 2. Method

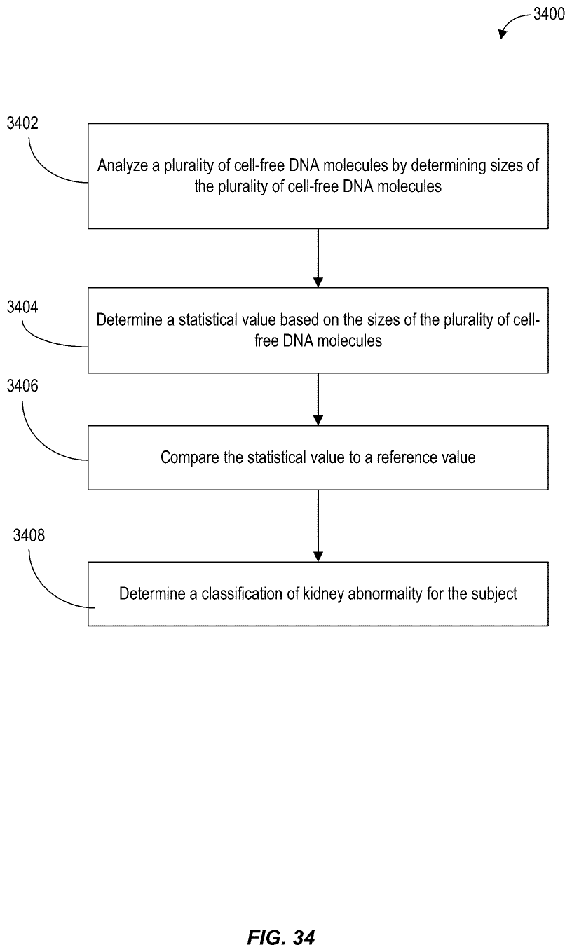

FIG. 34 shows a flowchart of a method 3400 for determining a classification of kidney abnormality based on sizes of urinary cell-free DNA, according to some embodiments. Aspects of method 3400 and other methods described herein may be performed in a similar manner as methods above, e.g., the sample preparation and analysis of DNA molecules. The urine sample may include a mixture of cell-free DNA molecules from one or more tissue types, such as heart, lungs, and liver. The urine sample may comprise tumor specific cell-free DNA molecules as well as other tissue-specific cell-free DNA molecules. For example, the urine sample can include cell-free DNA molecules specific to Renal Cell Carcinoma (RCC). Aspects of method 3400 and any other methods described herein may be performed by a computer system.

At block 3402, a plurality of cell-free DNA molecules from the urine sample are analyzed. Aspects of block 3402 may be performed in a similar manner as similar blocks of other methods, such as block 2102 of method 2100, as can be done for other methods herein. For example, the plurality of cell-free DNA molecules from the urine sample are analyzed to obtain sequence reads. As examples, the sequence reads can be obtained using sequencing or probe-based techniques, either of which may include enriching, e.g., via amplification or capture probes.

In some embodiments, analyzing the plurality of cell-free DNA molecules includes determining sizes of the plurality of cell-free DNA molecules. Various methods (e.g., gel electrophoresis) can be used to determine the sizes of the plurality of cell-free DNA molecules. For example, sizes of the plurality of cell-free DNA molecules can be measured using gel electrophoresis, filtration, size-selective precipitation, or hybridization. Additionally or alternatively, sizes of the plurality of cell-free DNA fragments can be measured using the sequence reads. For example, the sequence reads can be obtained from a sequencing (e.g., massively-parallel sequencing, single-molecule real-time sequencing, nanopore sequencing) of the plurality of cell-free DNA molecules from the biological sample. Then, to measure sizes of cell-free DNA molecules, a number of nucleotides can be counted for each sequence read. The skilled person will appreciate the variety of sequencing techniques that may be used. As part of the sequencing, it is possible that some of the sequence reads may correspond to cellular nucleic acids. In some instances, the urine sample can be enriched for DNA fragments having sizes that are less than a predefined size threshold (e.g., 80 bps).

A statistically significant number of cell-free DNA molecules can be analyzed as described herein.

At block 3404, a statistical value is determined for the set of cell-free DNA molecules. The statistical value can be determined based on the sizes of the plurality of cell-free DNA molecules. The size may form a size distribution. Various statistical values can be used, e.g., an average, mean, median, or mode of the size distribution can be used. As another example, the proportion of cfDNA in a first size range relative to a second size range can be used, where the size ranges are different but may overlap. The second size range may be all sizes, i.e., all cfDNA molecules.

In one example, a relative amount (example of a statistical value) of transrenal DNA in the urine samples can be characterized by DNA fragments with sizes less than 80 base pairs. If permeability of GBM is disturbed in some subjects, the relative amount of shorter DNA fragments in the urine sample can increase or decrease. If such changes relative to the normal amount exceeds a threshold (reference value), the subjects can be determined to have diseases or other abnormal conditions that affect permeability of the kidney (e.g., nephrotic syndrome, glomerulonephritis).

For example, the statistical value can be a size ratio of a first amount of cell-free DNA molecules that have sizes less than a size threshold (e.g., 80 bps) relative to a second amount corresponding to the plurality of cell-free DNA molecules. As examples, the size threshold can be 40 base pairs, 50 base pairs, 60 base pairs, 70 base pairs, 80 base pairs, 90 base pairs, 100 base pairs, 110 base pairs, 120 base pairs, 130 base pairs, 140 base pairs, 150 base pairs, or 160 base pairs, which may be used in any embodiment using a size threshold as described herein.

In some instances, determining the statistical value includes a proportion of a set of cell-free DNA molecules having sizes within a size range relative to the plurality of cell-free DNA molecules from the urine sample. The size range can have a lower bound and an upper bound, e.g., selected from 0, 5, 10, 15, 20, 30, 35, 40, 45, 50, 55, or 60 bases for the lower bound and any of 40, 50, 60, 70, 80, 90, 100, 110, 120, 130, 140, 150, or 160 bases.

At block 3406, the statistical value is compared to a reference value. The reference value can correspond to another statistical value determined based on measured sizes of cell-free DNA molecules of one or more reference samples, in which the one or more reference samples are associated with known classifications of the kidney abnormality. For example, the reference value can be determined based on sizes of the cell-free DNA molecules in healthy urine samples can be used. In some instances, the reference value is a calibration value or determined from calibration values of calibration (training) samples.

At block 3408, a classification of the subject having a kidney abnormality is determined based on the comparison. In some embodiments, comparing the statistical value to the reference value includes: (1) determining whether the statistical value differs from the reference value by at least a threshold amount or the difference is less than the threshold amount; (2) determining whether the statistical value is less than the reference value by at least a threshold amount; or (3) determining whether the statistical value is greater than the reference value by at least a threshold amount. The kidney abnormality can include renal cell carcinoma RCC, nephrotic syndrome, glomerulonephritis, Fabry disease, cystinosis, IgA nephropathy, IgM nephropathy, lupus nephritis, atypical hemolytic uremic syndrome (aHUS), polycystic kidney disease (PKD), Alport's syndrome, interstitial nephritis, proteinuria, chronic kidney disease, acute kidney injury, etc. In some instances, the classification of the subject having the kidney abnormality includes an increased level of permeability associated with a glomerular basement membrane of the kidney.

The classification of the kidney abnormality can be determined using machine learning trained using a training dataset. The training dataset can include training samples. The training samples can be associated with known classifications of the kidney abnormality. In another example, the comparison to the reference value can be performed using a machine learning model. The machine-learning model can be applied to the statistical value to generate the classification of the kidney abnormality. The machine learning models can include, but not limited to, convolutional neural network (CNN), linear regression, logistic regression, deep recurrent neural network (e.g., fully-connected recurrent neural network (RNN), Gated Recurrent Unit (GRU), long short-term memory, (LSTM)), transformer-based methods (e.g. XLNet, BERT, XLM, RoBERTa), Bayes's classifier, hidden Markov model (HMM), linear discriminant analysis (LDA), k-means clustering, density-based spatial clustering of applications with noise (DBSCAN), random forest algorithm, adaptive boosting (AdaBoost), eXtreme Gradient Boosting (XGBoost), support vector machine (SVM), or a composite model comprising one or more models proposed above.

C. Classification Based on Urinary cfDNA Concentration

We also evaluated the concentration difference of urinary cfDNA between healthy pregnant women and those with preeclampsia. Because the cfDNA concentration in a urine sample is dependent on the hydration status of the subject, the urinary cfDNA concentration was corrected (normalized).

In some embodiments, the correction of the urine concentration can use creatinine. For example, the amount of urinary DNA (e.g., as measured by mass per volume, such as ng/mL) can be corrected by the amount of creatinine (e.g., mmol). In one implementation, the corrected value was calculated by the urinary cfDNA concentration per milliliter of urine sample (e.g., determined by Qubit assay) divided by the concentration of creatinine, expressed as nanograms per milliliter of cfDNA per millimole of creatinine (ng/ml/mmol Cr). Creatinine is produced at a constant rate by muscle cells, and all creatinine filtered through glomeruli is excreted in urine. Therefore, the expression of urinary cfDNA concentration as per millimole of creatinine would minimize the variation of urinary cfDNA concentration arising from the difference in hydration status of the subjects.

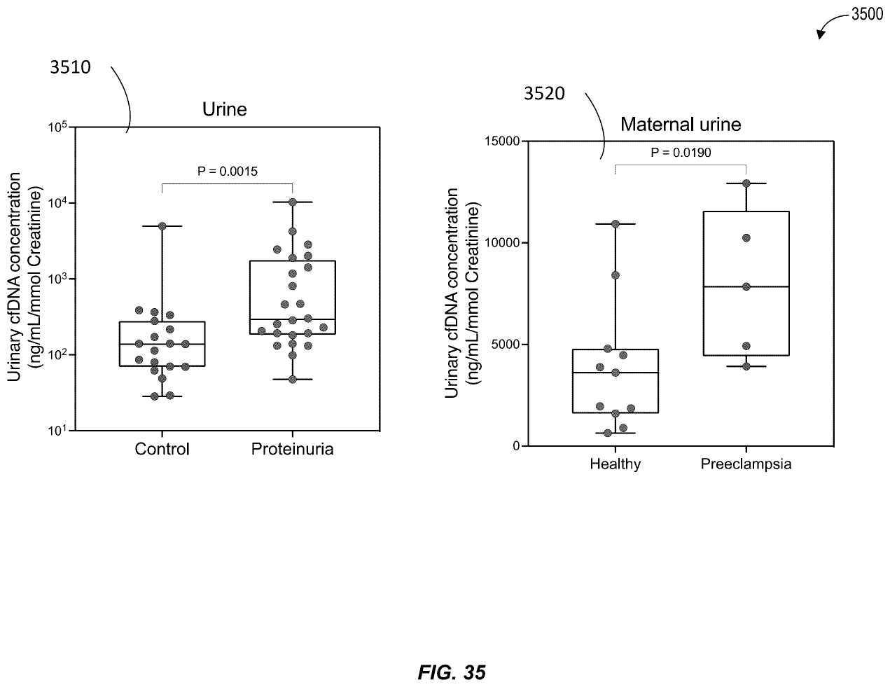

FIG. 35 shows urinary cfDNA concentration analysis of transrenal DNA in the patients with proteinuria and preeclampsia, respectively.

Boxplot 5310 shows the urinary cfDNA concentration in healthy controls and patients with proteinuria. We observed higher urinary cfDNA concentrations (Mann-Whitney U test, P-value=0.0015) in patients with proteinuria than in healthy controls.

Boxplot 3520 shows the urinary cfDNA concentration in healthy pregnant women and women with preeclampsia. We observed higher urinary cfDNA concentrations (Mann-Whitney U test, P-value=0.0190) in pregnant women with preeclampsia than in healthy pregnant women.

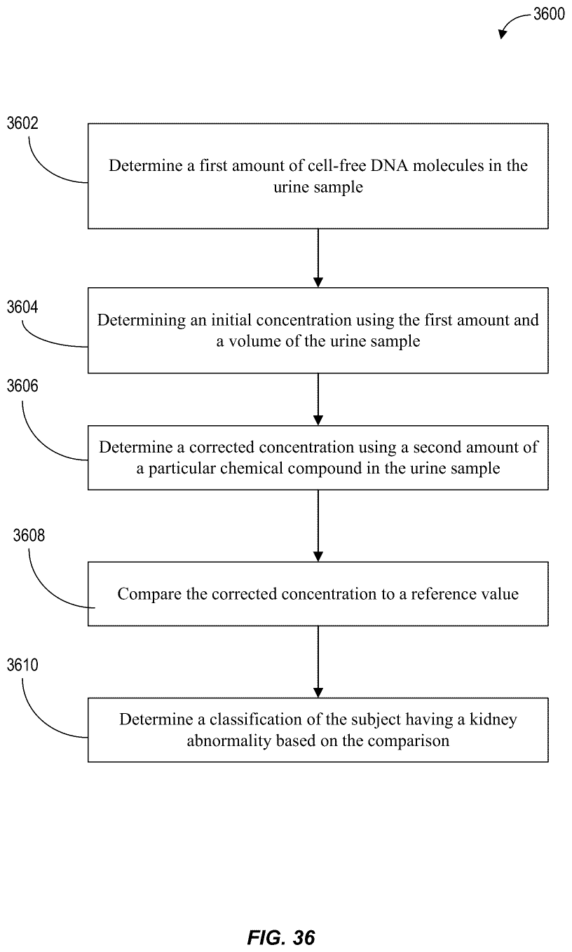

FIG. 36 is a flowchart of a method 3600 for determining a classification of kidney abnormality based on urinary cell-free DNA concentration, according to some embodiments. Method 3600 can detect a kidney abnormality using a urine sample of a subject, where the urine sample includes cell-free DNA molecules.

At block 3602, a first amount of cell-free DNA molecules in the urine sample is determined. As examples, the first amount can be determined using measurements using a fluorometer, a spectrophotometer, PCR, or sequencing. The first amount can be filtered so as to be of cfDNA molecules that satisfy one or more criteria. For instance, the cfDNA can be of a specified size, e.g., greater than a size cutoff, which may be between 40-200 bp. Examples of the size cutoff are provided herein and include 40, 50, 60, 70, 80, 90, 100, 110, 120, 130, 140, 150, 160, 170, 180, 190, or 200 bp. The specified size can be a size range with an upper and lower bound, just a lower bound, or just an upper bound.

At block 3604, an initial concentration is determined using the first amount and a volume of the urine sample. When size is used as a criteria, the initial concentration can be a proportion of cell-free DNA molecules in the urine sample that are within a specified range. The specified range can be greater than a size cutoff, e.g., as described above.

At block 3606, a corrected concentration is determined using a second amount of a particular chemical compound in the urine sample. The chemical compound can be a waste product of digestion, and thus be a natural-occurring chemical compound in a subject. As an example, the particular chemical compound can be creatinine. Creatinine is a waste product that comes from the digestion of protein in your food and the normal breakdown of muscle tissue, e.g., of creatine.

At block 3608, the corrected concentration is compared to a reference value. The reference value can be determined from one or more reference subjects for which a classification is known, e.g., presence or absence of the kidney abnormality or a particular severity of the kidney abnormality.

At block 3610, a classification of the subject having the kidney abnormality is determined based on the comparison. Examples of a kidney abnormality are provided herein and include preeclampsia and proteinuria.

Additional details of example ways for determining the first amount are provided. NanoDrop spectrophotometers are based on the principle that nucleic acids (i.e., DNA and RNA) absorb ultraviolet light with a peak at a wavelength of 260 nanometres (nm). A photo-detector measures the light that passes through the sample. The more light absorbed by the nucleic acids, the less light will strike the photodetector, producing a higher optical density (OD), resulting in higher nucleic acid concentration in a sample.

Qubit fluorometers quantify the DNA concentration by detecting fluorescent dyes in a sample. Fluorescent dyes specific for DNA substrate exhibit extremely low fluorescence before binding to the DNA target. Upon binding to DNA, the dye molecules increase fluorescence by several orders of magnitude through intercalation between the DNA bases.

Quantitative PCR (qPCR) assays quantify the DNA concentration by detecting the fluorescent signal of the DNA products during real-time PCR. QPCR monitors the amplification of targeted DNA molecules during the PCR by using fluorescent dyes or DNA probes labeled with a fluorescent reporter. As a result, the amount of amplified product is linked to fluorescence intensity.

Digital polymerase chain reaction (dPCR) assays involve partitioning the PCR solution into tens of thousands of nano-liter-sized droplets, where a separate PCR reaction of a single DNA molecule takes place in each one. The DNA probes with a fluorescent reporter would facilitate the detection of target DNA in a droplet and the fraction of the droplet containing target DNA can be translated into the DNA amounts. Further details can be found in the following three publications, which all use NanoDrop, Qubit, and qPCR for DNA concentration determination: Simbolo et al., PLOS ONE 2013; 8:e62692 Heydt et al., PLOS ONE 2014; 9:e104566 Ponti et al., Clinica Chimica Acta 2018; 479:14-19. Further details of dPCR for DNA concentration determination can be found in Gai et al., Clin. Chem. 2018; 64:1239-1249.

### D. Comparison of Techniques

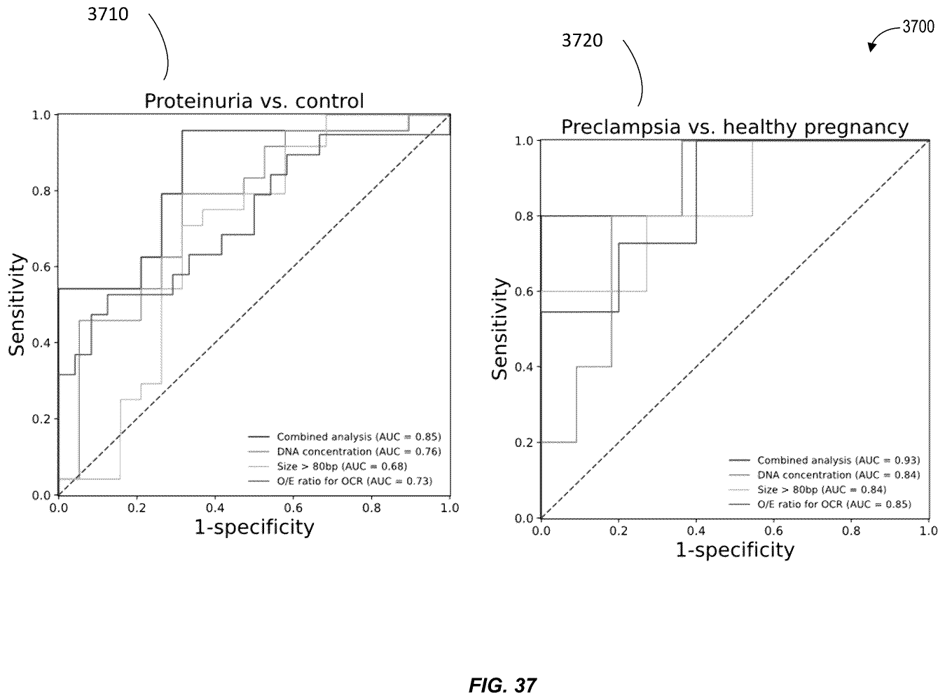

FIG. 37 shows an ROC analysis in differentiating patients with proteinuria and preeclampsia from healthy controls using fragmentomic features of transrenal DNA. FIG. 37 shows the ROC analysis using the techniques above.

ROC 3710 shows AUCs of 0.76, 0.68, 0.73, and 0.75 in differentiating patients with proteinuria from healthy controls using urinary cfDNA concentrations, sizes (i.e., >80 bp), and O/E in OCR (blood-specific DHSs). The AUC could be further improved to 0.85 in differentiating proteinuria by combining these fragmentomic features with a support vector machine (SVM) method.

We further performed the ROC analysis on these samples, in which the AUC was 0.84, 0.84, and 0.85 in differentiating pregnant women with preeclampsia from healthy pregnant subjects using urinary DNA concentration, cfDNA sizes (i.e., >80 bp), and O/E in OCR (placenta-specific DHSs), respectively. When these three fragmentomic features were combined using a SVM, one could observe an improved performance in differentiating between preeclampsia and healthy subjects (AUC: 0.93).

The SVM provides a separation of samples in a higher dimensions. The number of features input to the SVM would provide the number of dimensions in the SVM. In the examples above, we used three features, and thus three dimensions were used. Additional features can be used, resulting in more than three dimensions.

### VIII. F-PROFILES OF CFDNA AND NUCLEASE ACTIVITY

Different types of cell-free DNA cleavage were linked to different fragmentation processes, including enzymatic and non-enzymatic breakages. There are techniques that focus on one specific nuclease activity each time using one end motif or several top-ranked end motif. Such approaches can be effective but may fail to provide a comprehensive view of nuclease activities occurring in a given sample (e.g., plasma sample, urine sample).

To address the above deficiencies, a number of nuclease activities or other fragmentation processes can be assessed simultaneously using deduced relative contributions concerning the different types of cell-free DNA cleavage. For example, relative frequencies of DNA molecules corresponding to 256 end motifs can be determined for a subject with a known disease diagnosis (e.g., HCC). The relative frequencies of DNA molecules can be factorized to a set of “F-profiles” that identify the relationship of ending sequences (e.g., 1-30 bases) of cell-free DNA fragments (also just referred to as DNA fragments) in the sample. The set of F-profiles can then be used in deconvolution of relative frequencies of DNA molecules obtained from another subject to predict fraction of clinically-relevant DNA molecules, a classification of a disease, etc.

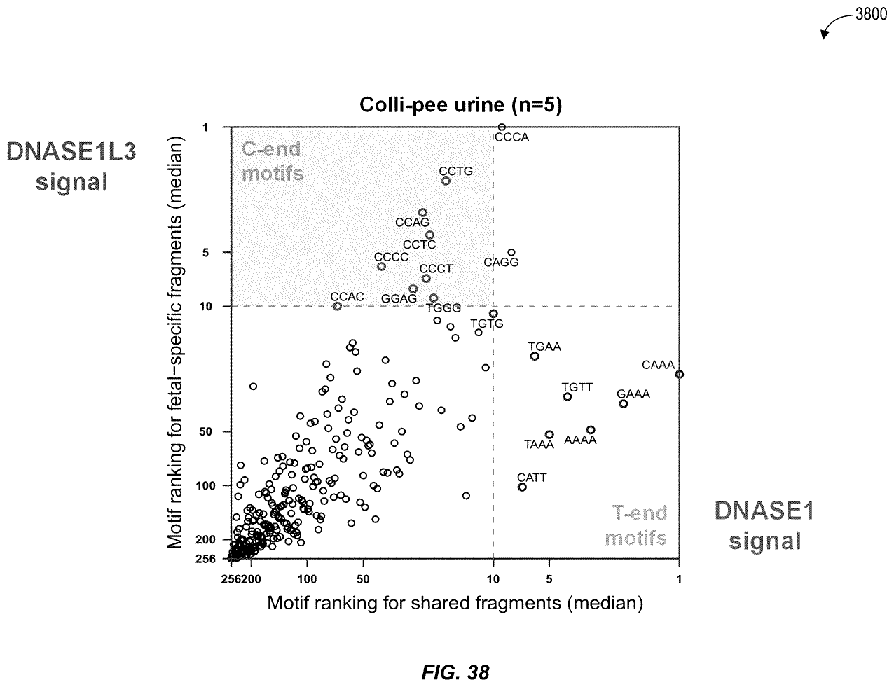

FIG. 38 shows a plot graph 3800 that identifies a ranking of frequencies of certain end motifs present in urinary cell-free DNA molecules. FIG. 38 corresponds to FIG. 9. As shown in FIG. 38, the fetal-specific transrenal DNA predominantly include fragments with C-ends, which are typically associated with DNASE1L3 cutting preference. The shared non-transrenal DNA predominantly include fragments with T-ends, which are typically associated with DNASE1 cutting preference.

Although focusing on certain end motifs can be beneficial in determining fetal DNA (for example), the plot graph 3800 shows additional end-motif information that may provide further insight: relative frequencies of DNA molecules across most of 256 end-motifs are different between fetal-specific DNA and shared DNA. Thus, it can be advantageous to incorporate the relative frequencies of DNA molecules across all 256 end motifs to determine fetal DNA fraction or determine a disease classification for a subject.

### A. End-Motif Profile Characteristics Across Different Murine Samples

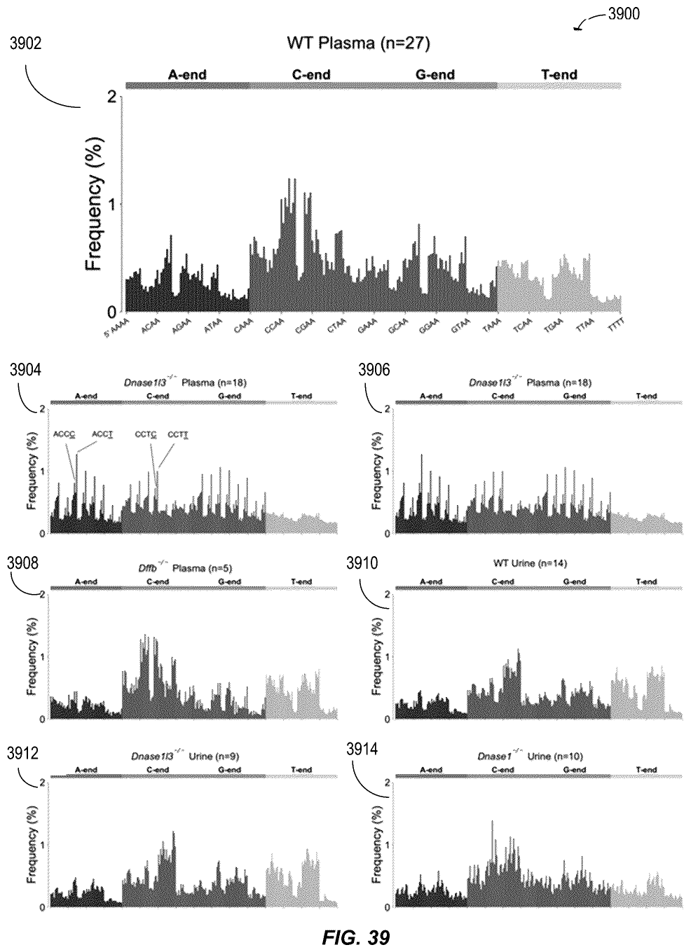

FIG. 39 shows a set of graphs 3900 that identify observed end-motif profiles of murine plasma and urinary cell-free DNA molecules. As shown in FIG. 39, the frequencies of 256 4-mer end motifs in both plasma and urinary cell-free DNA of mice with different nuclease knockout genotypes were organized in alphabetical order, forming the end-motif profile. Motifs starting with adenine (A), cytosine (C), guanine (G), and thymine (T) were highlighted in blue, red, green, and yellow, respectively.

We observed certain distinct patterns in end-motif profiles across different mice. The observed end-motif frequencies of plasma cell-free DNA from WT mice, Dnase1l3^{-/-} mice, Dnase1^{-/-} mice, and Dffb^{-/-} mice are shown in graphs 3902, 3904, 3906 and 3908, respectively. The observed end-motif frequencies of urinary cell-free DNA from WT mice, Dnase1l3^{-/-} mice, and Dnase1^{-/-} mice are shown in graphs 3910, 3912, and 3914, respectively.

Compared with WT mice, the plasma cell-free DNA of the Dnase1l3^{-/-} mice showed periodic spikes in the end-motif profile, typically at those end motifs with A-end, C-end, and G-end. For urinary cell-free DNA of the WT mice, the abundance of motifs with T-end was elevated significantly (P<0.0001, Mann-Whitney U test), compared with the Dnase1^{-/-} mice. Although it was visually hard to discern the difference when comparing plasma cell-free DNA of WT mice versus Dnase1^{-/-} mice, or urinary cell-free DNA of WT mice versus Dnase1l3^{-/-} mice, we hypothesized that the subtle differences in 256-dimension end-motif profiles could be depicted when reference profiles were used, e.g., via a decomposition (factorization) into the reference profiles. In some embodiments, non-negative matrix factorization (NMF) was used to consider 256 motifs as a whole instead of focusing on one or a few specific motif species.

An end-motif profile can be a K-mer where K can have various values, e.g., 1, 2, 3, 4, 5, 6, or more. As shown in FIG. 39, K of 4 is used.

B. NMF for Determining F-Profiles of Urinary cfDNA

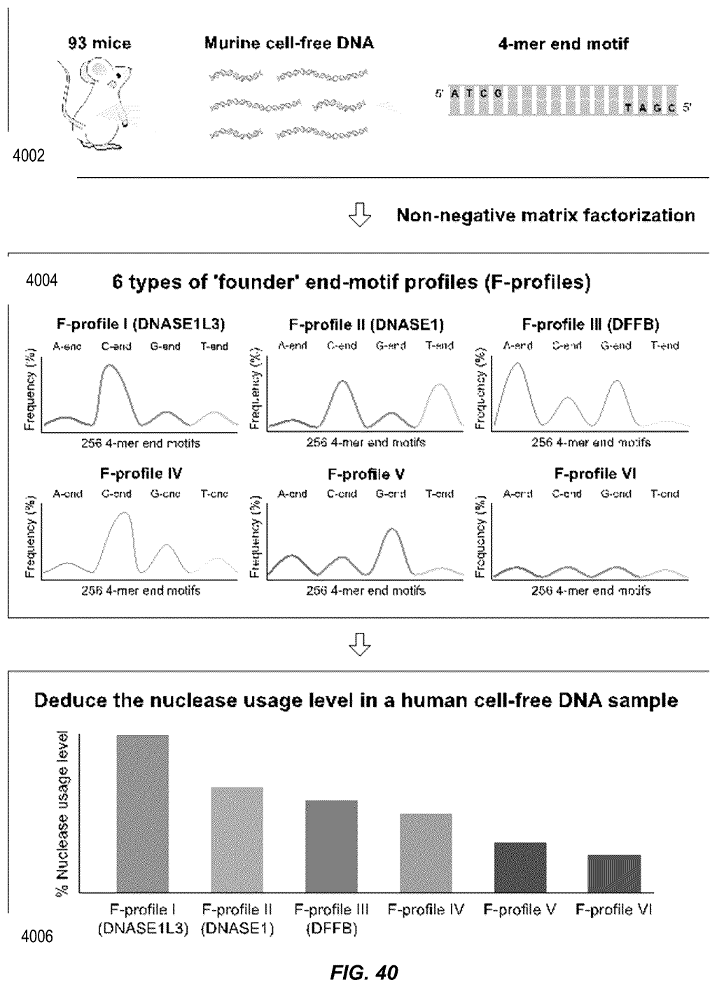

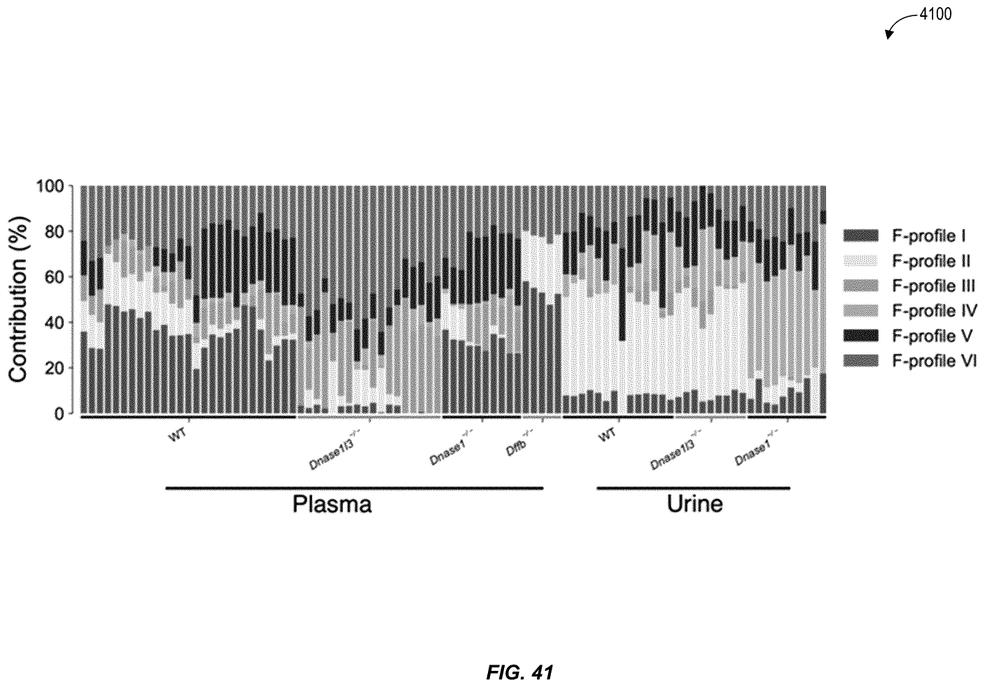

FIG. 40 shows a schematic workflow of an example nuclease-usage level analysis for cell-free DNA molecules. 93 murine cell-free DNA samples were sequenced, including 60 plasma cell-free DNA samples and 33 urinary cell-free DNA samples. The mouse plasma cell-free DNA samples were taken from 27 WT mice, 10 mice with Dnase1 gene deletion (Dnase1), 18 mice with Dnase1l3 gene deletion (Dnase1l3^{-/-}), 5 mice with Dffb gene deletion (Dffb^{-/-}), with a median number of paired-end reads of 50 million (range: 16-243 million). In addition, whole-genome sequencing data of mouse urinary cell-free DNA samples were obtained from 14 WT mice, 10 Dnase1^{-/-} mice, and 9 Dnase1l3^{-/-} mice (median number of paired-end reads: 43 million; range: 2-134 million).

At block 4002, the terminal 4 nucleotides at each of the 5′ fragment ends (i.e., 4-mer end motifs; n=256) were determined for 93 murine cell-free DNA samples, including WT mice and nuclease-deficient mice (e.g., Dnase1l3^{-/-}, Dnase1^{-/-}, Dffb^{-/-}).

For each murine sample, 256 4-mer end motifs of cell-free DNA molecules were then used to infer their respective nuclease usage levels.

At block 4004, six categories of reference end-motif profiles, referred to as the F-profiles, were determined from the cell-free DNA molecules for each murine sample. In some embodiments, the relative frequencies of DNA molecules ending with 4-mer end-motifs were subjected to non-negative matrix factorization (NMF) analysis to determine the underlying different types of cell-free DNA cleavage.

We applied NMF (Daniel et al. Nature 1999; 401:788-791; Stein-O'Brien et al. Trends Genet. 2018; 34:790-805) analysis to decompose the relative frequencies of the cell-free DNA molecules into several F-profiles. A total of 93 murine cell-free DNA samples with different genotypes of DNA nuclease knockouts were used for such NMF analysis, including 60 plasma cell-free DNA samples and 33 urinary cell-free DNA samples. After obtaining the end-motif frequencies, a data matrix (M) was constructed in a way that each row indicates a cell-free DNA sample (a total of 93 murine cell-free DNA samples) and each column represents a type of end motif (a total of 256 end motifs), thus having the dimension of 93×256. The data matrix was subjected to NMF analysis for obtaining two matrices Wand F. The mathematical relationship among M, W, and F were shown below:

M=WF.

M was the result of the product of W and F where W was the relative weight for each F-profile in a 93×n matrix, where n corresponded to the number of F-profiles. F represented the F-profiles in an n×256 matrix. W and F were determined by minimizing the objective function below:

∥M−WF∥, subject to W≥0 and F≥0.

Singular value decomposition (SVD) was used to initialize the procedure of NMF. Such factorization analysis was implemented in the Python language by using the function of sklearn.decomposition.NMF (v1.1.1) (Pedregosa et al. J. Mach. Learn. Res. 2011; 12:2825-2830).

To estimate the optimal number of F-profiles, a 5-fold cross-validation pre-analysis was performed. Such factorization analysis could yield a number of different types of cell-free DNA cleavage. In this example, six F-profiles (namely F-profiles I, II, III, IV, V, and VI) were determined by considering the tradeoff between the reproducibility of factorized components and the value of objective function (i.e., end-motif profile reconstruction error). The number of different types of cell-free DNA cleavage could be, but not limited to, 2, 3, 4, 5, 6, 7, 8, 9, 10, 15, 20, 30, 40, 50, 100, etc, with corresponding number of reference end-motif profiles. In FIG. 40, F-profiles I, II, and III can be associated with the cutting preference of DNASE1, DNASE1L3, and DFFB, respectively.

At block 4006, the nuclease usage analysis framework learned from murine cell-free DNA could be extrapolated to human cell-free DNA analysis for informing the proportional contributions of different nuclease activities in both murine and human cell-free DNA samples. An observed end motif profile can be reconstructed by iteratively adjusting the proportional contribution of each F-profile. In other words, with the use of the F-profiles generated from cell-free DNA of mice, the proportional contributions of F-profiles for any cell-free DNA sample. In some embodiments, such deduced proportional contributions of F-profiles are used to reflect the nuclease activities or nuclease usage levels in any cell-free DNA sample.

Additionally or alternatively, such deduced proportional contributions of F-profiles could be used to reflect other types of fragmentations that might be involved in a patient, such as but not limited to, oxidative stress-induced DNA damage, drug treatment-induced DNA damage, radioactivity-induced DNA damage, etc. The presence, absence, and alterations of an F-profile contribution could be suggestive of having diseases or being at risk of developing diseases. In some embodiments, another mathematics algorithm is for factorization such as but not limited to the component principal analysis (PCA), t-distributed stochastic neighbor embedding, uniform manifold approximation and projection, etc.

### C. F-Profiles in Plasma and Urine Samples

FIG. 41 shows a diagram 4100 that identifies proportional contribution of each F-profile (i.e., nuclease usage level) deduced from murine cell-free DNA samples with different knockout genotypes using NMF analysis. Each F-profile can indicate a pattern of relative frequencies of cell-free DNA molecules across the 256 end motifs. The six F-profiles can be used as a signature of end-motif frequencies of cell-free DNA molecules for the corresponding sample. The proportional contribution of each F-profile in an individual cell-free DNA sample could be determined when the minimal error was achieved between an observed end-motif profile and the sum of F-profiles weighted by their proportional contributions.

As shown in FIG. 41, the proportional contributions of F-profiles in murine samples tend to share similarities based on their respective nuclease activity levels. For example, WT samples showed a substantial contribution of F-profile I, whereas the Dnase1l3^{-/-} samples showed almost no contribution of F-profile I. In another example, the Dnase1^{-/-} samples showed significantly less contribution of F-profile II, and Dffb^{-/-} samples showed almost no contribution of F-profiles III, IV, and V. Using the similar patterns of the F-profile contributions, one can assess nuclease activity levels of another sample from a test subject.

### D. Fragmentomic Characteristics of F-Profiles

As described above, the six F-profiles are linked to possible DNA nuclease activities. To illustrate such feasibility, we investigated the typical end motifs in an F-profile and measured its alteration in proportional contribution when depleting or enhancing a particular nuclease activity.

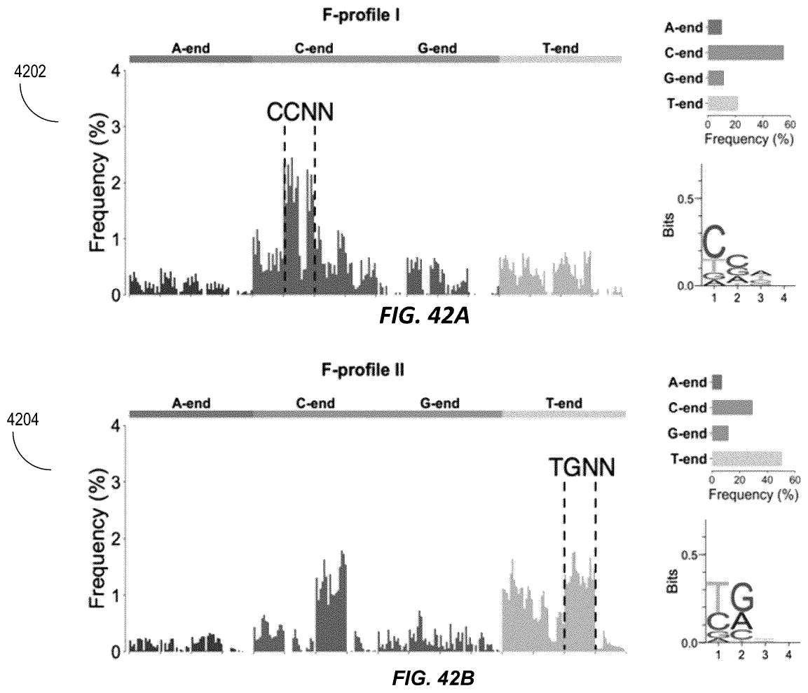

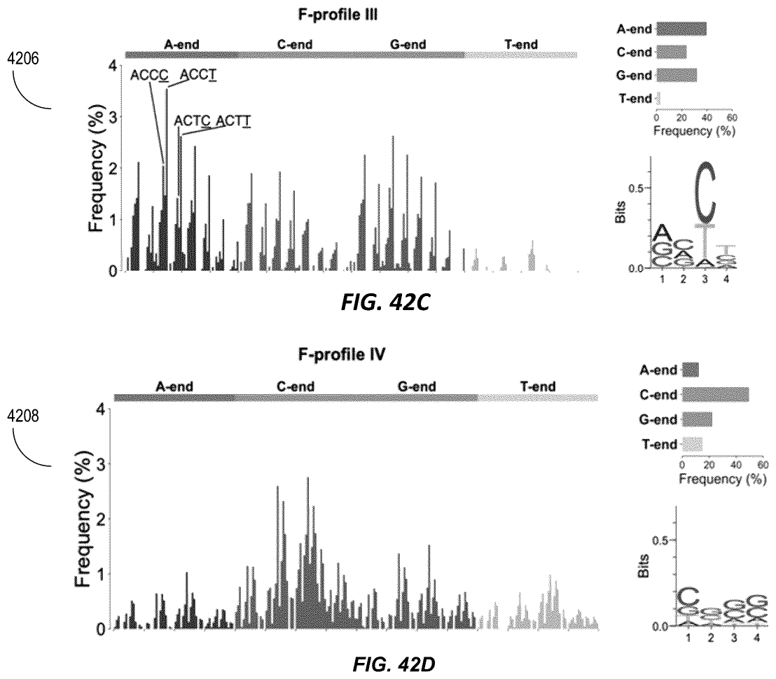

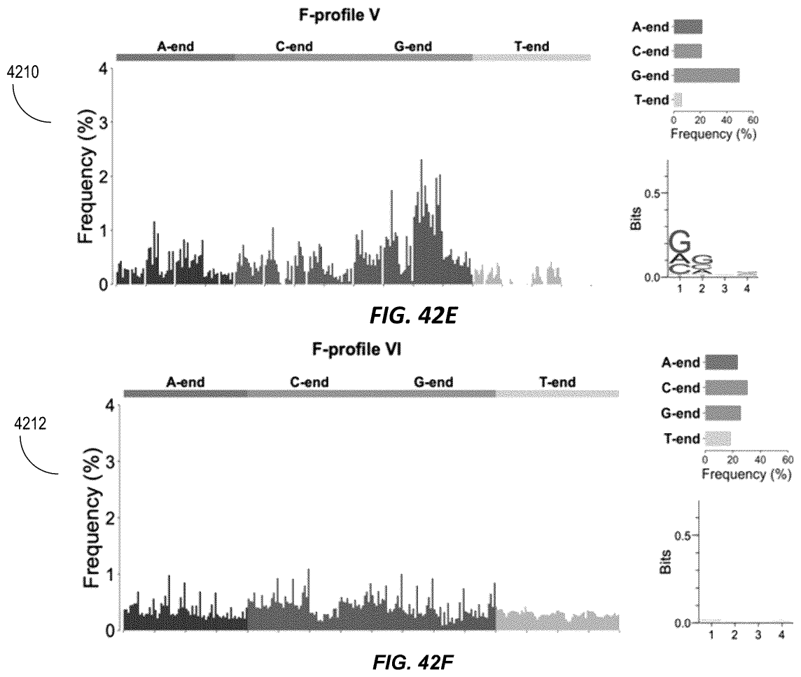

FIG. 42 shows a set of plots for six F-profiles (A-F) deduced from mouse plasma and urinary cell-free DNA using NMF analysis. Each of the F-profiles plots 4202-4212 contains the 4-mer end motif profile, 1-mer end motif frequencies, and sequence preference at each position of the 4-mer end motif. F-profile 14202 displayed a predominance of C-end motifs (55%) and was characterized by the “CC” started motifs, which was in line with DNASE1L3-cutting properties demonstrated in our previous studies (Serpas et al. Proc Natl Acad Sci USA. 2019; 116:641-649; Jiang et al. Cancer Discov. 2020; 10:664-73). We observed that the contributions of F-profile I in the plasma cell-free DNA of Dnase1l3^{-/-} mice were significantly lower compared with that in WT mice (median: 2.7% versus 35.4%; range: 0.0-4.6% versus 19.5-47.9%) (P<0.0001, Mann-Whitney U test). Hence, F-profile I was deemed to be a DNASE1L3-associated F-profile, which could be used to reflect the nuclease usage level of DNASE1L3.

F-profile II 4204 exhibited a major preference for T-end motifs (51%) with a preference for “TG” started motifs. In WT mice, F-profile II contributions were significantly higher in urinary cell-free DNA in comparison with plasma cell-free DNA (median: 43.4% versus 11.6%; range: 31.8-50.1% versus 0.0-22.1%) (P<0.0001, Mann-Whitney U test). Of note, the DNASE1 activity was much higher in urine than in plasma for WT mice (Chen et al. PLOS Genet. 2022; 18:e1010262). Furthermore, there was a median of approximately 8-fold reduction for F-profile II contributions in both plasma and urinary cell-free DNA of Dnase1^{-/-} mice compared with the WT counterparts. Thus, F-profile II was deduced to be related to the DNASE1 activity.

F-profile III 4206 included a substantial proportion of A-end motifs (40%) and was characterized by the preference for C and T nucleotides at the third and fourth positions in the 4-mer motifs, respectively, in the 5′ to 3′ direction. The contributions of F-profile III reduced significantly in the plasma cell-free DNA of Dffb^{-/-} mice (median: 0.0%; range: 0.0-0.5%) compared with their counterparts in WT mice (median: 10.1%; range: 0.0-26.9%) (P=0.0004, Mann-Whitney U test). Therefore, F-profile III was considered to be associated with DFFB activity.

Although F-profile IV 4208 exhibited a high C-end preference (50%) which was to some extent reminiscent of F-profile I, it had several distinct characteristics, e.g., the absence of CC-end preference. F-profile IV also exhibited “G” base preferences at the second, third, and fourth positions in 4-mer motifs. F-profile V 4210 exhibited a strong G-end preference (50%). These results suggested that F-profile IV and V were not directly attributed to the known nucleases involved in cell-free DNA fragmentation, implying that some other enzymatic and/or non-enzymatic processes might play roles in the cell-free DNA fragmentation processes. In addition, F-profile VI 4212 showed a relatively even distribution across 256 motifs without obvious sequence preference, suggesting one possibility that the other DNA nucleases or other factors might also cause non-specific cleavages.

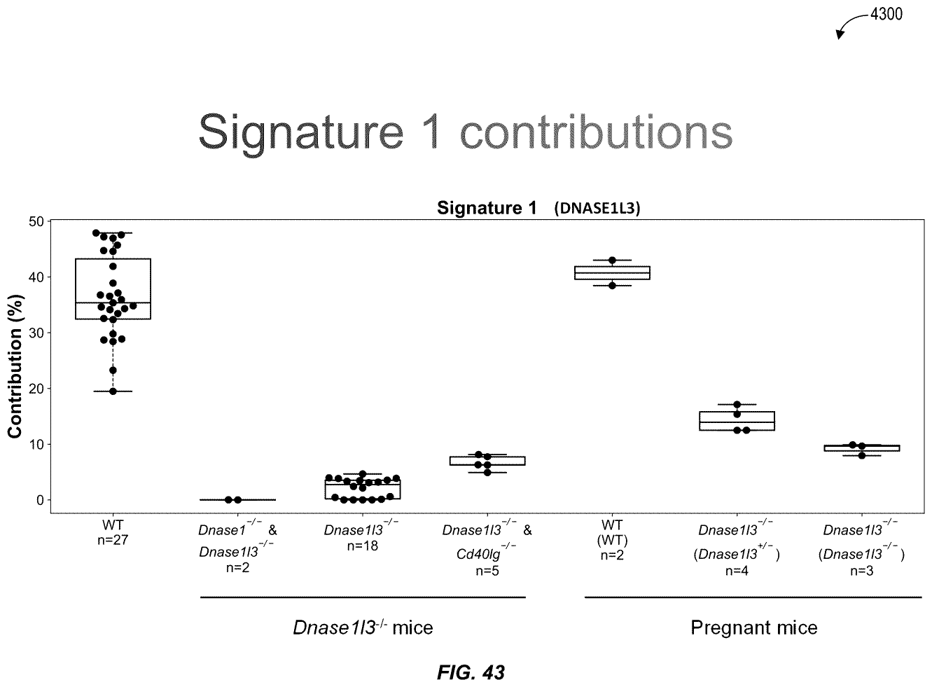

FIG. 43 shows a boxplot 4300 that identifies proportional contribution of F-profile I across different types of samples, according to some embodiments. The x-axis shows cell-free DNA molecules obtained from different types of samples including wildtype (WT) murine samples (n=27), Dnase1^{-/-} and Dnase1l3^{-/-} murine samples (n=2), Dnase1l3^{-/-} murine samples (n=18), and Dnase1l3^{-/-} and Cd40lg^{-/-} murine samples (n=5). In addition, the x-axis further shows various types of pregnant murine samples, including WT murine samples (n=2), Dnase1l3^{-/-} maternal sample in which fetal DNA exhibit Dnase1l3^{-/-} (n=4), and Dnase1l3^{-/-} maternal sample in which fetal DNA exhibit Dnase1l3^{-/-} (n=3). The proportional contribution of F-profile I was determined relative to contributions from other F-profiles II-VI.

As shown in FIG. 43, the proportional contributions of F-profile I significantly decreased for murine samples in which DNASE1L3 were knocked out. Moreover, the significant decrease of F-profile I contribution appears similar for pregnant samples as well. Interestingly, F-profile I contribution for the fetal Dnase1l3^{-/-} sample shows a slight increase over F-profile I contribution for the fetal Dnase1l3^{-/-} sample. Based on the proportional contributions of F-profile I, it can be determined that the F-Profile I corresponds to a signature associated with DNASE1L3.

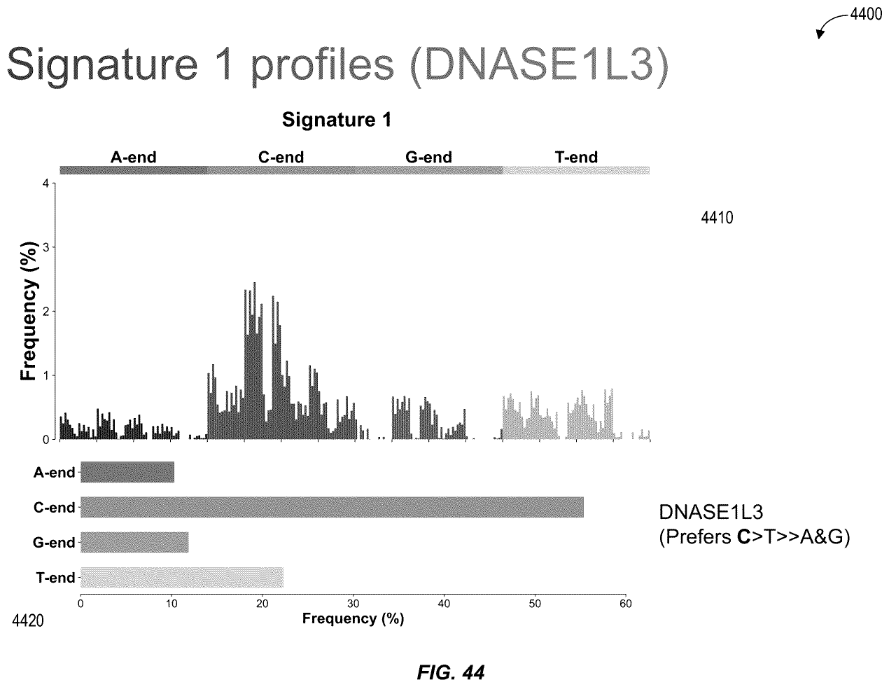

FIG. 44 shows relative frequencies 4400 of cell-free DNA molecules across 256 end motifs for F-profile I, according to some embodiments. Plot 4410 shows an end-motif profile using 4-mers as the end motif. Plot 4420 shows an end-motif profile using 1-mers (single nucleotide) as the end motif. As shown in the plots of FIG. 44, the F-profile I has a cutting preference of the C-ends, followed by T-ends, and A- and G-ends. This cutting preference is substantially similar to cutting preference of DNASE1L3, which supports the findings shown in FIG. 43.

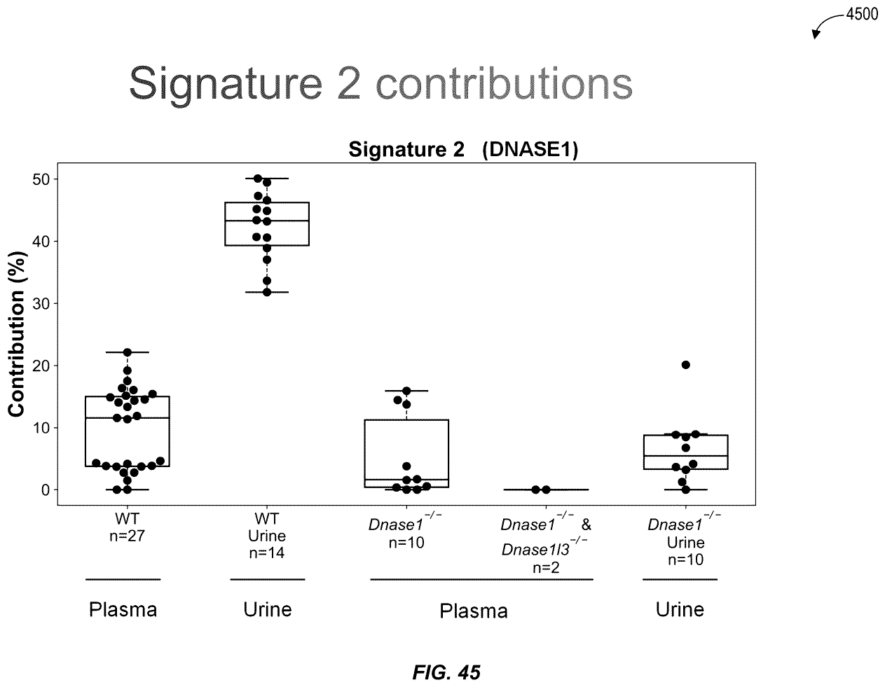

FIG. 45 shows a boxplot 4500 that identifies proportional contribution of F-profile II across different types of samples, according to some embodiments. The x-axis shows cell-free DNA molecules obtained from different types of murine samples including wildtype (WT) plasma samples (n=27), WT urine samples (n=14), Dnase1^{-/-} plasma samples (n=10), Dnase1/Dnase1l3 double deletion plasma samples (n=2), and Dnase1^{-/-} urine samples (n=10). The proportional contribution of F-profile II was determined relative to contributions from other F-profiles I and III-VI.

As shown in FIG. 45, the proportional contributions of F-profile II are significantly high for urine samples compared to plasma samples. Further, the proportional contributions of F-profile II do not show significant changes across different types of plasma samples. However, the proportional contributions of F-profile II of Dnase1^{-/-} urine samples exhibit a significant decrease from WT urine samples. Based on the proportional contributions of F-profile II, it can be determined that the F-Profile II corresponds to a signature associated with DNASE1. Indeed, DNase1 nuclease activity appears to be more active in urine samples, which correlates with high F-profile II contribution for WT urine samples.

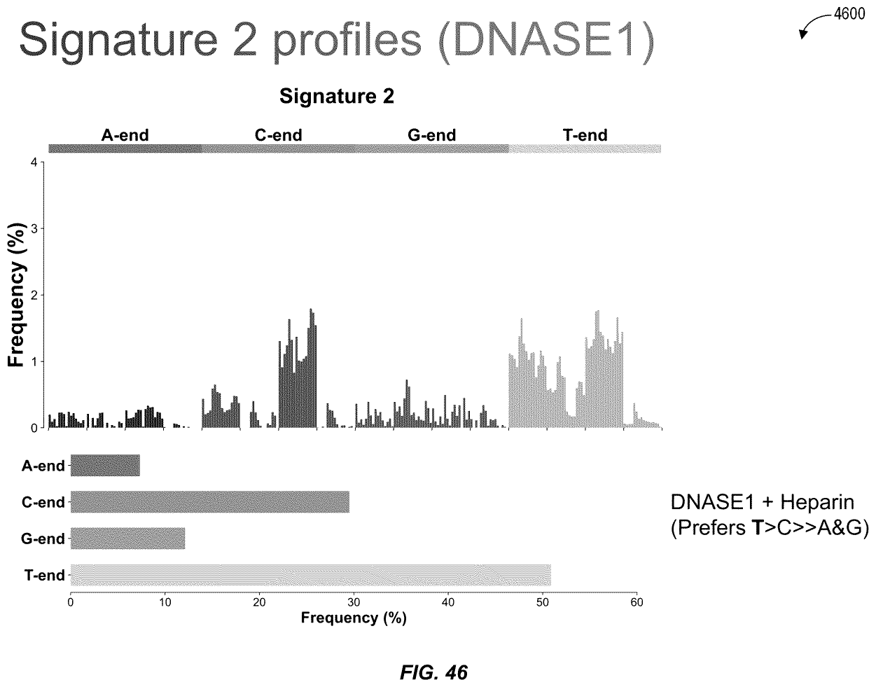

FIG. 46 shows relative frequencies 4600 of cell-free DNA molecules across 256 end motifs for F-profile II, according to some embodiments. As shown in FIG. 46, the F-profile II has a cutting preference of the T-ends, followed by C-ends, and A- and G-ends. This cutting preference is substantially similar to cutting preference of DNASE1, which supports the findings shown in FIG. 45.

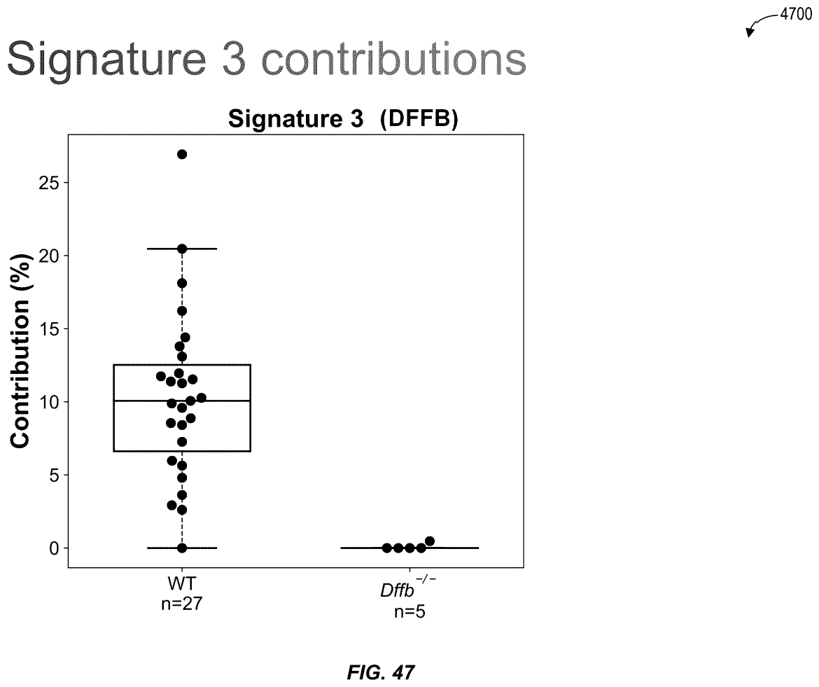

FIG. 47 shows a boxplot 4700 that identifies proportional contribution of F-profile III across different types of samples, according to some embodiments. The x-axis shows cell-free DNA molecules obtained from including wildtype (WT) samples (n=27) and Dffb^{-/-} samples (n=5). The proportional contributions of F-profile III was determined relative to contributions from other F-profiles I, II, and IV-VI. As shown in FIG. 47, the proportional contributions of F-profile III significantly decreased for Dffb^{-/-} samples, relative to that of WT samples. Based on the proportional contributions of F-profile III, it can be determined that the F-Profile III corresponds to a signature associated with DFFB.

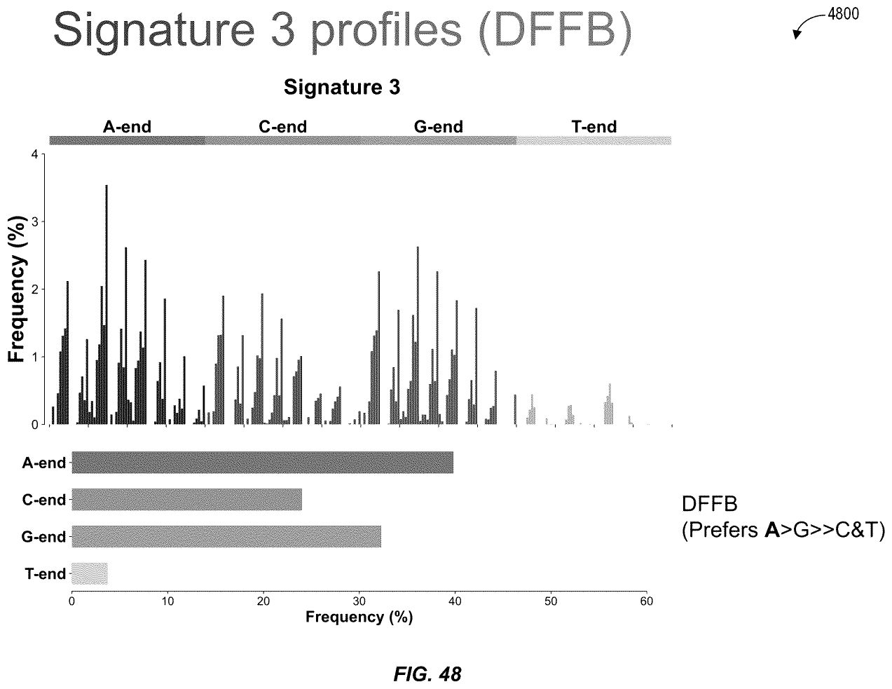

FIG. 48 shows relative frequencies 4800 of cell-free DNA molecules across 256 end motifs for F-profile III, according to some embodiments. As shown in FIG. 48, the F-profile III has a cutting preference of the A-ends, followed by G-ends, and C- and T-ends. This cutting preference is substantially similar to cutting preference of DFFB, which supports the findings shown in FIG. 47.

In addition to F-profiles I-III, we also decompose F-profiles IV-VI.

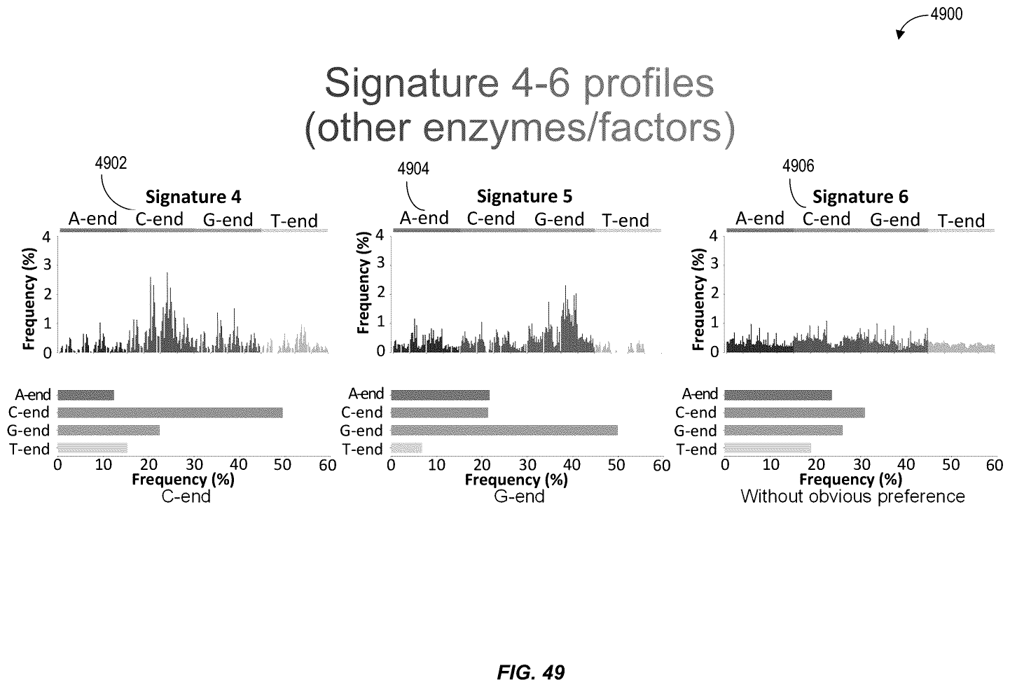

FIG. 49 shows relative frequencies 4900 of cell-free DNA molecules across 256 end motifs for F-profiles VI-VI, according to some embodiments. Each of the F-profiles IV-VI shows its own cutting preference. For example, F-profile IV 4902 exhibits a cutting preferences for C-ends, F-profile V 4904 exhibits a cutting preferences for G-ends, and F-profile VI 4906 exhibits no particular cutting preferences. Based on the above, it can be shown that F-profile VI may be associated with cutting patterns not caused by a particular nuclease that we have investigated, but other types of fragmentation factors.

E. Analysis of F-Profiles Across Different Samples from Human Subjects

We then explored whether the murine F-profiles of DNASE-mediated cell-free DNA cleavages can be applied for human subjects.

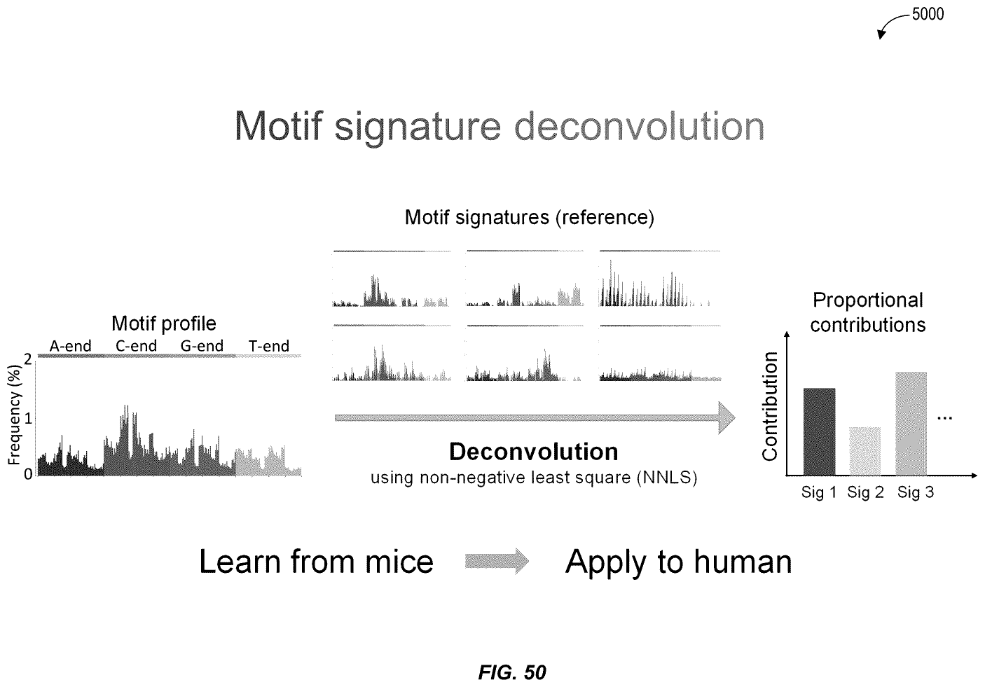

FIG. 50 shows a schematic diagram 5000 of comparing an end-motif profile of a human subject to reference F-profiles determined based on murine samples, according to some embodiments. To make the motif patterns directly comparable between human and mice, the frequencies of 4-mer end motifs related to the human and murine cell-free DNA can be normalized by the genomic contexts of the human and mouse genomes, respectively. For example, an expected 4-mer end-motif frequency can be used for the normalization step, in which the expected end-motif frequency was determined by simulating 4-mer end motifs from a reference genome using a 4-bp sliding window across each chromosome. The normalized end motif frequency was calculated as a ratio of observed and expected frequencies and then divided by the sum of all 256 normalized motif frequencies. The total normalized end motif frequency can be equal to 100%. The end motif frequency mentioned in this NMF-based nuclease usage analysis was termed the normalized end motif frequency.

Once the normalization is complete, proportional contributions of the F-profiles can be determined for the normalized end frequencies of the human sample. The proportional contributions can be determined by applying deconvolution to the normalized end frequencies. For example, a data matrix M of dimensions W by F can be used, in which: (i) M can represent the normalized end frequencies across 256 end motifs for a given biological sample; (ii) F can represent end frequencies of the reference F-profiles obtained from murine samples; and (iii) W can represent relative weights corresponding to the proportional contributions of each F-profile.

The F end frequencies can be determined based on the proportions of the cell-free DNA molecules of the set of reference F-profiles. The proportional contributions can be determined by solving for the W relative weights based on using non-negative least square (NNLS) on values from the data matrix M and the reference F-profiles. The proportional contributions determined using deconvolution can be used to identify an extent of nuclease activity levels (e.g., relative decrease of F-profile I contribution) in certain human biological samples.

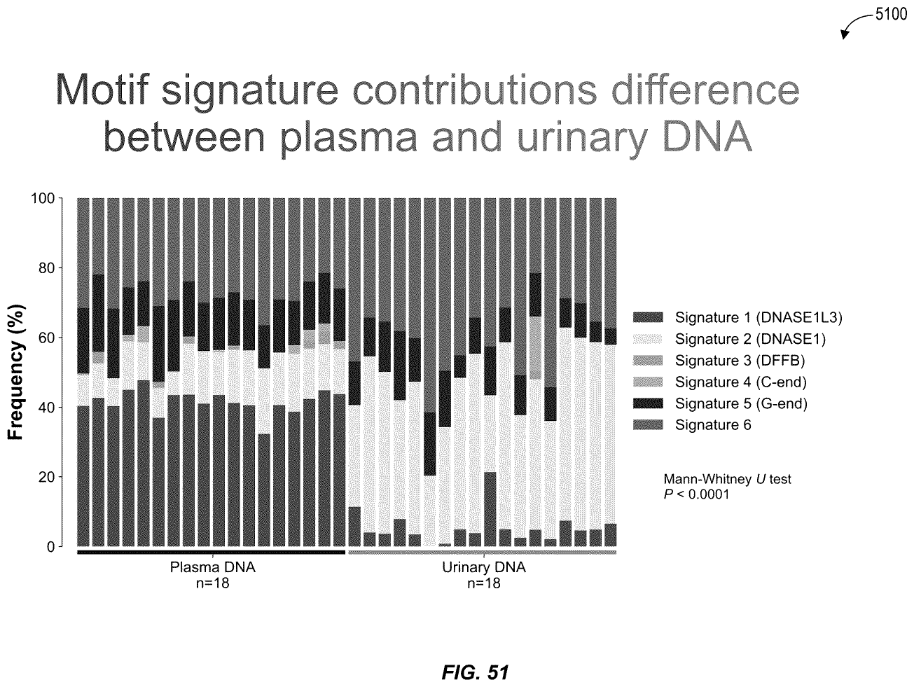

FIG. 51 shows proportional contributions 5100 of F-profiles across plasma and urine samples of human subjects, according to some embodiments. First, the deconvolution process in FIG. 51 was applied to plasma and urine samples from human subjects. As shown in FIG. 51, the plasma samples included a relatively high contribution from F-profile I, which has been associated with DNASE1L3 activity. By contrast, the urine samples included a relatively high contribution from F-profile II, which has been associated with DNASE1 activity.

The data shown in FIG. 51 is consistent with experiments that DNASE1L3 has a major contribution to fragmentation patterns of cell-free DNA molecules in plasma samples and that DNASE1 has a major contribution to fragmentation patterns of cell-free DNA molecules in urine samples. Accordingly, it has been shown that the deconvolution process using reference F-profiles from murine samples can be effectively used in identifying fragment cutting patterns of cell-free DNA molecules in human samples.

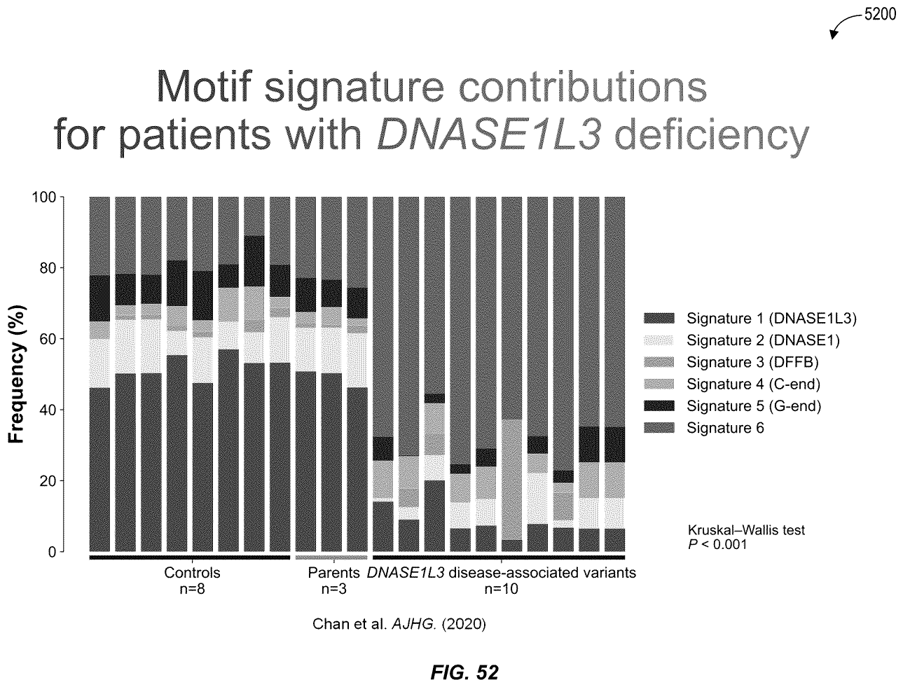

FIG. 52 shows proportional contributions 5200 of F-profiles across normal and DNASE1L3-deficient samples of human subjects, according to some embodiments. The deconvolution process in FIG. 52 was applied to normal and DNASE1L3-deficient samples from human subjects. As shown in FIG. 52, the plasma samples from control subjects included a relatively high contribution (approximately greater than 40%) of F-profile I. By contrast, the DNASE1L3-deficient samples included an significantly lower contribution (approximately less than 15%) from F-profile I. The data shown in FIG. 52 is consistent with the previous correlation of F-profile I with nuclease activities of DNASE1L3. It can be shown that the deconvolution process using reference F-profiles from murine samples can be effectively used in identifying fragment cutting patterns of cell-free DNA molecules in human samples.

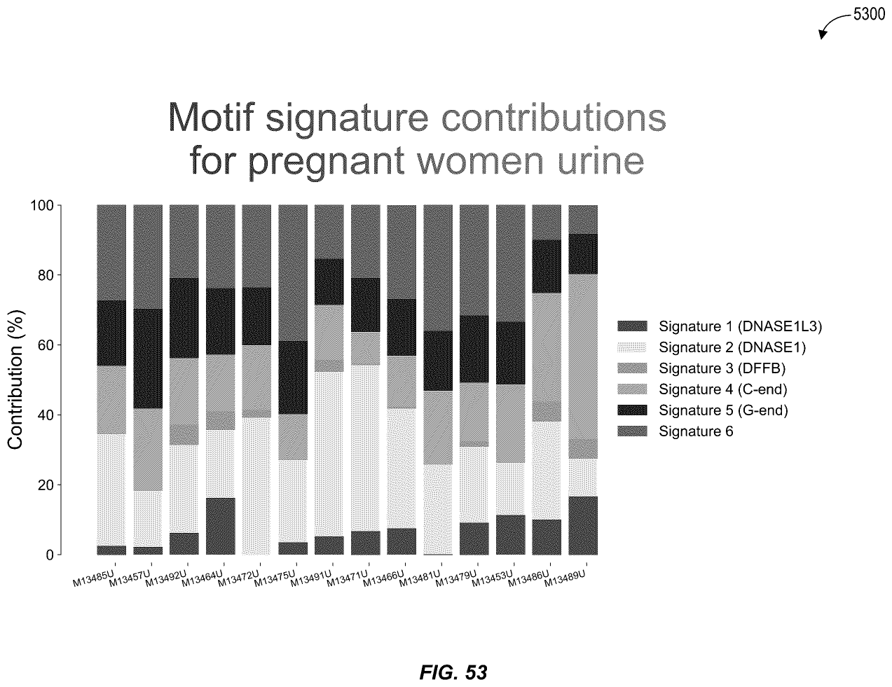

FIG. 53 shows proportional contributions 5300 of F-profiles across urine samples of pregnant human subjects, according to some embodiments. The deconvolution process in FIG. 42 was applied to the samples from pregnant human subjects. As shown in FIG. 53, the urine samples included a relatively high contribution from F-profile II, which has been associated with DNASE1 activity. The data shown in FIG. 53 is consistent with experiments that DNASE1 has a major contribution to fragmentation patterns of cell-free DNA molecules in urine samples. Moreover, the higher proportional contribution of F-profile IV may be indicative of a relatively high number of cell-free DNA fragments having C-ends. This observation is consistent with the plot data in FIG. 38 that fetal DNA in urine samples of pregnant subjects includes a larger proportion of cell-free DNA molecules having C-ends.

### F. Methods for Classifying Nuclease Activity Using F-Profiles

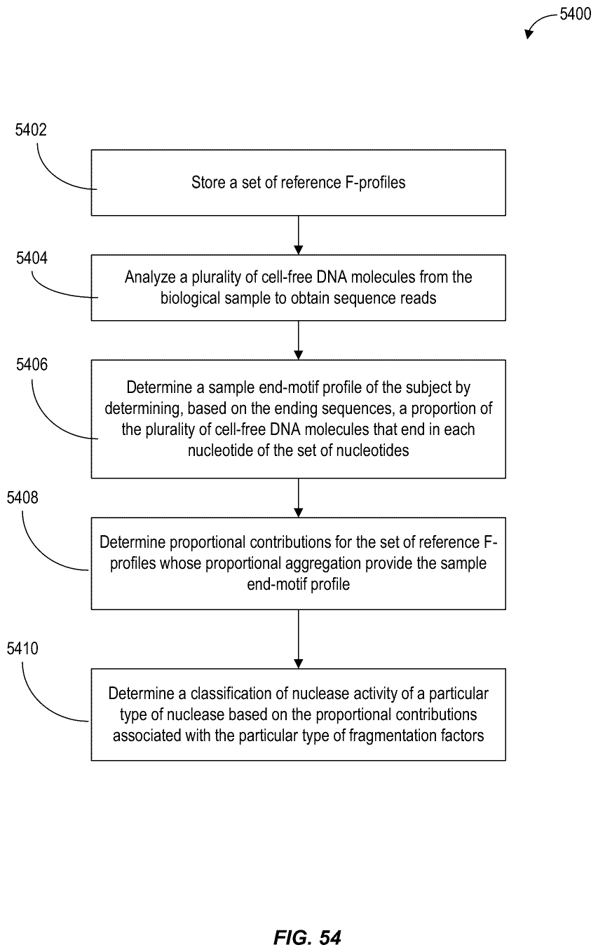

FIG. 54 shows a flowchart of a method 5400 for determining a classification of nuclease activity based on F-profiles of cell-free DNA molecules, according to some embodiments. Example biological samples can be cell-free samples that include cell-free DNA, e.g., blood, plasma, serum, urine, and saliva.

At block 5402, a set of reference F-profiles are stored. Each reference F-profile of the set identifies, for each nucleotide of a set of nucleotide, a proportion of cell-free DNA molecules that end in the nucleotide. In addition, each reference F-profile is associated with a type of fragmentation factors. The type of fragmentation factor identifies a particular enzyme (e.g., DNASE1L3, DNASE1), protein (e.g., DFFB), or other biological components or processes that cause fragmentation in cell-free DNA molecules. In some instances, the set of reference F-profiles include 1, 2, 3, 4, 5, 6, 7, 8, 9, 10, 15, 20, 25, 30, 40, 45, 50, or more than 50 F-profiles. For example, the set of reference F-profiles can include six F-profiles I-VI.

Each reference F-profile and sample end-motif profile can have a separate proportion for each K-mer end motif of a set of K-mer end motifs. For example, FIG. 39 shows a profile where K=4, resulting in 256 different proportion values. Plot 4420 of FIG. 44 shows a profile where K=1, resulting in 4 different proportion values. Accordingly, each reference F-profile of the set of reference F-profiles can specify the proportion of cell-free DNA molecules that end in each K-mer end motif of a set of K-mer end motifs, wherein K is one or two or more.

In some instances, the set of reference F-profiles are determined using one or more reference samples. The reference samples can be obtained from non-human subjects (e.g., murine samples) whose classification of genetic disorders are known (e.g., WT, DNASE1L3^{-/-}, DNASE1^{-/-}). To determine a reference F-profile of the set of reference profiles, a factorization algorithm (e.g., NMF, PCA) is used to decompose the relative frequencies of the cell-free DNA molecules of the reference samples into several F-profiles. For example, reference cell-free DNA samples with different genotypes of DNA nuclease knockouts were selected. After obtaining the end-motif frequencies of the reference samples, a data matrix (M) is constructed in a way that each row indicates a cell-free DNA sample (e.g., a total of 93 murine cell-free DNA samples) and each column represents a type of end motif (e.g., a total of 256 4-mer end motifs), thus having the dimension of 93×256. The data matrix can then be subjected to NMF analysis for obtaining two matrices Wand F.

M=WF.

M is the result of the product of W and F where W was the relative weight for each F-profile in a 93×n matrix, where n corresponded to the number of F-profiles. F represented the F-profiles in an n×256 matrix. W and F were determined by minimizing the objective function below:

∥M−WF∥, subject to W≥0 and F≥0.

At block 5404, a plurality of cell-free DNA molecules from the biological sample are analyzed to obtain sequence reads. The sequence reads include ending sequences corresponding to ends of the plurality of cell-free DNA molecules. The sequence reads can include ending sequences corresponding to ends of the plurality of cell-free DNA fragments. As examples, the sequence reads can be obtained using sequencing or probe-based techniques, either of which may including enriching, e.g., via amplification or capture probes.

The sequencing may be performed in a variety of ways, e.g., using massively parallel sequencing or next-generation sequencing, using single molecule sequencing, and/or using double- or single-stranded DNA sequencing library preparation protocols. The skilled person will appreciate the variety of sequencing techniques that may be used. As part of the sequencing, it is possible that some of the sequence reads may correspond to cellular nucleic acids.

The sequencing may be targeted sequencing as described herein. For example, biological sample can be enriched for DNA fragments from a particular region. The enriching can include using capture probes that bind to a portion of, or an entire genome, e.g., as defined by a reference genome.

A statistically significant number of cell-free DNA molecules can be analyzed so as to provide an accurate determination of the fractional concentration. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed.

At block 5406, a sample end-motif profile of the subject is determined by determining, based on the ending sequences, a proportion of the plurality of cell-free DNA molecules that end in each nucleotide of the set of nucleotides. The sample end-motif profile identifies relative frequencies of a plurality of end motifs corresponding to the ending sequences of the plurality of cell-free DNA molecules. The plurality of end motifs can correspond to all feasible combinations of N base positions. For example, if the plurality of end motifs correspond to 4-mers, the plurality of end motifs of the sample end-motif profile can include 256 4-mer combinations (e.g., CCCA, TTCC).

To determine the sample end-motif profile, for each of the plurality of cell-free DNA molecules, an end motif is determined for each of one or more ending sequences of the cell-free DNA molecules. The end motifs can include N base positions (e.g., 1, 2, 3, 4, 5, 6, etc.). As examples, the end motif can be determined by analyzing the sequence read at an end corresponding to the end of the DNA molecule, correlating a signal with a particular motif (e.g., when a probe is used), and/or aligning a sequence read to a reference genome.

For example, after sequencing by a sequencing device, the sequence reads may be received by a computer system, which may be communicably coupled to a sequencing device that performed the sequencing, e.g., via wired or wireless communications or via a detachable memory device. In some implementations, one or more sequence reads that include both ends of the nucleic acid fragment are received. The location of a DNA molecule can be determined by mapping (aligning) the one or more sequence reads of the DNA molecule to respective parts of the human genome, e.g., to specific regions. Additionally or alternatively, a particular probe (e.g., following PCR or other amplification) can indicate a location or a particular end motif, such as via a particular fluorescent color. The identification can be that the cell-free DNA molecule corresponds to one of the plurality of end motifs.

Then, relative frequencies of the plurality of end motifs corresponding to the ending sequences of the plurality of cell-free DNA molecules is determined to determine the sample end-motif profile of the subject. A relative frequency of a sequence motif can provide a proportion of the plurality of cell-free DNA molecules that have an ending sequence corresponding to the sequence motif.

At block 5408, proportional contributions for the set of reference F-profiles is determined whose proportional aggregation provide the sample end-motif profile. The proportional contributions of the set of reference F-profiles sum to one. The proportional contributions can be determined by applying deconvolution to the sample end-motif profile of the subject. For example, a data matrix M of dimensions W by F can be used, in which: (i) M can represent the normalized end frequencies across 256 end motifs for the sample end-motif profile; (ii) F can represent end frequencies of the reference F-profiles obtained from murine samples; and (iii) W can represent relative weights corresponding to the proportional contributions of each F-profile. The proportional contributions can be determined by solving for W based on using non-negative least square (NNLS) on values from the data matrix M and reference F-profiles F. The proportional contributions determined using deconvolution can be used to identify a level of fragmentation factor activity (e.g., relative decrease of F-profile I contribution) in the subject.

In some instances, frequencies of 4-mer end motifs of the sample end-motif profile of the subject (e.g., a human subject) and those of reference samples (e.g., murine samples) are normalized by the genomic contexts of their respective genomes. For example, an expected 4-mer end-motif frequency can be used for the normalization step, in which the expected end-motif frequency was determined by simulating 4-mer end motifs from a reference genome using a 4-bp sliding window across each chromosome. The normalized end motif frequency was calculated as a ratio of observed and expected frequencies and then divided by the sum of all 256 normalized motif frequencies. The total normalized end motif frequency can be equal to 100%.

At block 5410, a classification of nuclease activity of a particular type of nuclease is determined based on the proportional contributions associated with the particular type of fragmentation factors. For example, the classification of nuclease activity of a particular type of nuclease can include a classification of decreased nuclease activity associated with the particular nuclease. The classification of nuclease activity can be used to determine a classification of whether the subject has a nuclease activity deficiency, or a genetic disorder for a gene associated with a nuclease. The genetic disorder may be a disorder of the DNASE1L3 gene. Genetic disorders may include disorders of one or more of the following genes: DNASE1, DFFB, TREX1 (Three Prime Repair Exonuclease 1), AEN (Apoptosis Enhancing Nuclease), EXO1 (Exonuclease 1), DNASE2 (Deoxyribonuclease 2), ENDOG (Endonuclease G), APEX1 (Apurinic/Apyrimidinic Endodeoxyribonuclease 1), FEN1 (Flap Structure-Specific Endonuclease 1), DNASE1L1 (Deoxyribonuclease 1 Like 1), DNASE1L2 (Deoxyribonuclease 1 Like 2), and EXOG (Exo/Endonuclease G).

In some instances, a decreased level of nuclease activity associated with the particular type of nuclease is determined based on the proportional contributions of the of the set of reference F-profiles. For example, a proportional contribution associated with one of the set of reference F-profiles can be compared to a cutoff value. Based on the comparison (e.g., if the proportional contribution exceeds the cutoff value), a decreased level of nuclease activity can be determined. In some instances, the cutoff value is determined using one or more reference samples with known classifications of the nuclease activity.

### IX. FRACTIONAL CONCENTRATION USING F-PROFILES OF CFDNA

Since the transrenal cell-free DNA molecules still preserve the DNASE1L3 cutting signature of plasma cell-free DNA, we reasoned that the nuclease usage levels in the urine could potentially represent the transrenal cell-free DNA amount. We hypothesized that the NMF-based nuclease usage level analysis could be feasible for determining the fractional contribution of transrenal cell-free DNA in urine samples. To this end, we applied nuclease usage level analysis to 14 maternal urine samples.

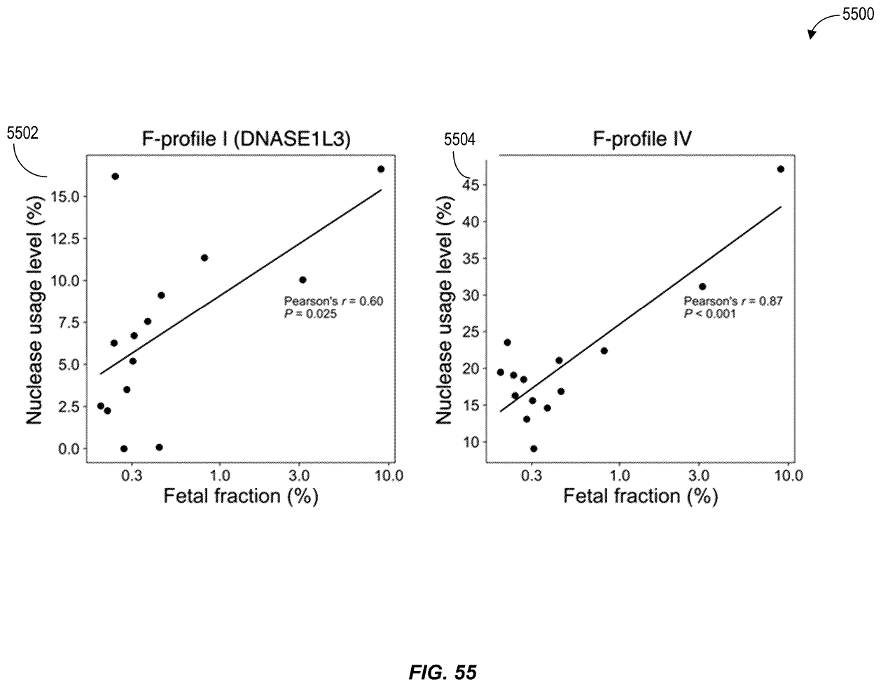

FIG. 55 shows a set of graphs 5500 that identify the nuclease usage levels in urinary cell-free DNA of pregnant women. A graph 5502 shows correlations between the fetal DNA fractions and F-profile I (DNASE1L3) levels. A graph 5504 shows correlations between the fetal DNA fractions and F-profile IV levels. As shown in FIG. 55, we found that the proportional contributions of F-profile I (DNASE1L3) and F-profile IV were significantly correlated with fetal DNA fractions estimated by an SNP-based approach (Pearson's r=0.60, P=0.025) in maternal urine cell-free DNA. Hence, the nuclease usage level analysis present in this disclosure may be useful for monitoring transrenal cell-free DNA proportion in urine samples.

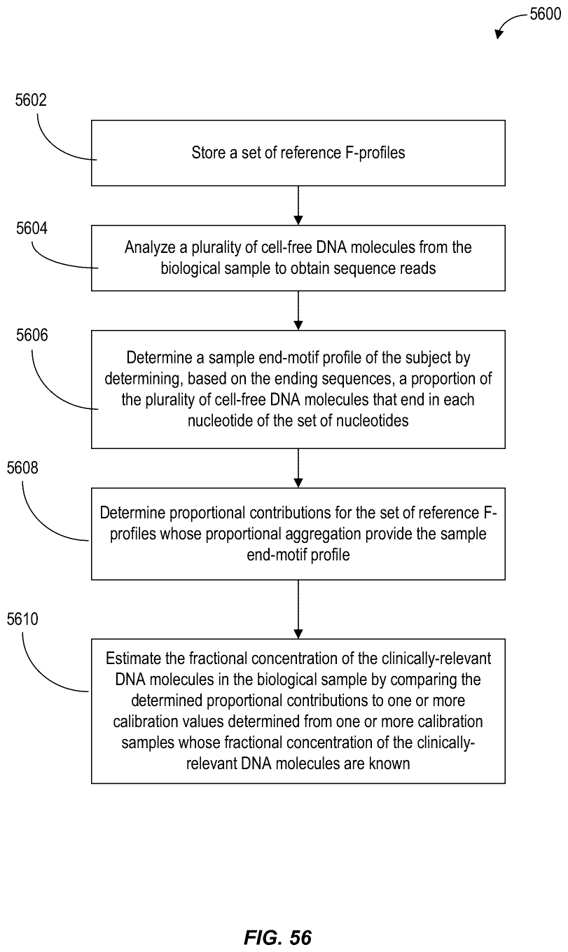

FIG. 56 shows a flowchart of a method 5600 for determining fractional concentration of fetal DNA based on F-profiles of cell-free DNA molecules, according to some embodiments. Example biological samples can be cell-free samples that include cell-free DNA, e.g., blood, plasma, serum, urine, and saliva. The biological sample can include the clinically-relevant DNA and other DNA that are cell-free. In other examples, a biological sample may not include the clinically-relevant DNA, and the estimated fractional concentration may indicate zero or a low percentage of the clinically-relevant DNA.

The biological sample may include a mixture of cell-free DNA molecules from one or more tissue types, such as heart, lungs, and liver. For example, the biological sample may be obtained from a pregnant woman comprising maternal cell-free DNA molecules and fetal cell-free DNA molecules. The biological sample may comprise tumor specific cell-free DNA molecules as well as other tissue-specific cell-free DNA molecules. The clinically-relevant DNA molecules may be of any of the tissue types described herein, e.g., fetal DNA, tumor DNA, or transplant DNA. Aspects of method 5600 and any other methods described herein may be performed by a computer system. Aspects of method 5600 may be performed in a similar manner as method 5400 of FIG. 54.

At block 5602, a set of reference F-profiles are stored. Each reference F-profile of the set can identify, for each nucleotide of a set of nucleotide, a proportion of cell-free DNA molecules that end in the nucleotide. In addition, each reference F-profile can be associated with a type of fragmentation factors. The type of fragmentation factor can identify a particular enzyme (e.g., DNASE1L3, DNASE1), protein (e.g., DFFB), or other biological components or processes that cause fragmentation in cell-free DNA molecules. In some instances, the set of reference F-profiles include 1, 2, 3, 4, 5, 6, 7, 8, 9, 10, 15, 20, 25, 30, 40, 45, 50, or more than 50 F-profiles. For example, the set of reference F-profiles can include six F-profiles I-VI. Block 5602 may be performed in a similar manner as block 5402 of FIG. 54.

At block 5604, a plurality of cell-free DNA molecules from the biological sample are analyzed to obtain sequence reads. The sequence reads include ending sequences corresponding to ends of the plurality of cell-free DNA molecules. The sequence reads can include ending sequences corresponding to ends of the plurality of cell-free DNA fragments. Block 5604 may be performed in a similar manner as block 5404 of FIG. 54.

At block 5606, a sample end-motif profile of the subject is determined by determining, based on the ending sequences, a proportion of the plurality of cell-free DNA molecules that end in each nucleotide of the set of nucleotides. The sample end-motif profile identifies relative frequencies of a plurality of end motifs corresponding to the ending sequences of the plurality of cell-free DNA molecules. The plurality of end motifs can correspond to all feasible combinations of N base positions. For example, if the plurality of end motifs correspond to 4-mers, the plurality of end motifs of the sample end-motif profile can include 256 4-mer combinations (e.g., CCCA, TTCC). Block 5606 may be performed in a similar manner as block 5406 of FIG. 54.

At block 5608, proportional contributions for the set of reference F-profiles are determined whose proportional aggregation provide the sample end-motif profile. The proportional contributions of the set of reference F-profiles sum to one. Block 5608 may be performed in a similar manner as block 5408 of FIG. 54.

The set of reference F-profiles include a first reference F-profile that correlates with the fractional concentration of the clinically-relevant DNA molecules, e.g., as determined using calibration samples whose fractional concentration is known. FIG. 55 provides such an examples, where the first reference F-profile is F-profile I (which corresponds to DNASE1L3) or F-profile IV. A first proportional contribution for the set of reference F-profiles can correspond to the first reference F-profile.

At block 5610, the fractional concentration of the clinically-relevant DNA molecules in the biological sample is estimated by comparing the first proportional contribution corresponding to the first reference F-profile to one or more calibration values determined from one or more calibration samples whose fractional concentration of the clinically-relevant DNA molecules are known. Aspects of block 5610 can be performed in a similar manner as block 2106 of method 2100. The first reference F-profile can correspond to a particular type of nuclease, e.g., DNASE1L3.

As shown in FIG. 55, the fetal DNA fraction increases as the proportional contribution(s) of one or more reference F-profiles (e.g., F-profile I, F-profile IV) increase. Any of the one or more reference F-profiles can be the first reference F-profile, as long as it correlates to the fractional concentration of the clinically-relevant DNA. Accordingly, known proportional contribution(s) of one or more reference F-profiles in the calibration samples can be used as calibration data points for determining fractional concentration of the clinically-relevant DNA molecules in the biological sample.

Some embodiments can, for each calibration sample of the one or more calibration samples, measure the fractional concentration of the clinically-relevant DNA molecules in the calibration sample and measure a proportional contribution of the first reference end-motif profile for the calibration sample, thereby determining one or more calibration data points. The proportional contribution of the first reference end-motif profile can be used as a calibration value. Proportional contribution of all of the set of references F-profiles can be determined, thereby determining multiple calibration values, e.g., when multiple reference F-profiles are used to estimate the fractional concentration. The skilled person will appreciate that the fractional concentrations can be measured in various ways, some of which are described herein, e.g., using a tissue-specific allele or a tissue-specific methylation pattern.

In some instances, the proportional contribution(s) determined for the one or more of the reference F-profiles (including the first proportional contribution for the first reference F-profile) are compared to a calibration curve (composed of the calibration data points), and thus the comparison can identify the point on the curve having the known proportional contributions for the biological sample. The fractional concentration corresponding to the identified point can then be used to estimate the fractional concentration. For example, the determined proportional contributions can be provided as an input to the calibration function (e.g., a linear or non-linear fit) to obtain an output of the fractional concentration.

In some embodiments, multiple proportional contributions can be used, as mentioned above. In such an instance, a calibration curve can be a calibration surface in two or more dimensions. Accordingly, estimating the fractional concentration of the clinically-relevant DNA molecules in the biological sample can include comparing one or more additional proportional contributions to one or more additional calibration values determined from the one or more calibration samples whose fractional concentration of the clinically-relevant DNA molecules are known.

### X. ESTIMATING GESTATIONAL AGE BASED ON F-PROFILES IN CF DNA

The nuclease usage levels identified from the factorization analysis of cell-free DNA molecules can be used to estimate gestational age of fetuses in samples obtained form pregnant women. For example, the proportional contributions of F-profiles that are obtained from samples of known gestational ages can be determined. The determined proportional contributions can then be used as calibration data points to estimate a gestational age for another pregnant sample.

As further described below, there was a correlation between proportional contributions of F-profile I and gestational ages of the fetus. The correlation can also indicate that the gestational age can be affected based on activity levels of DNASE1L3, since F-profile I represents the cutting preferences of DNASE1L3.

### A. Estimating Gestational Age

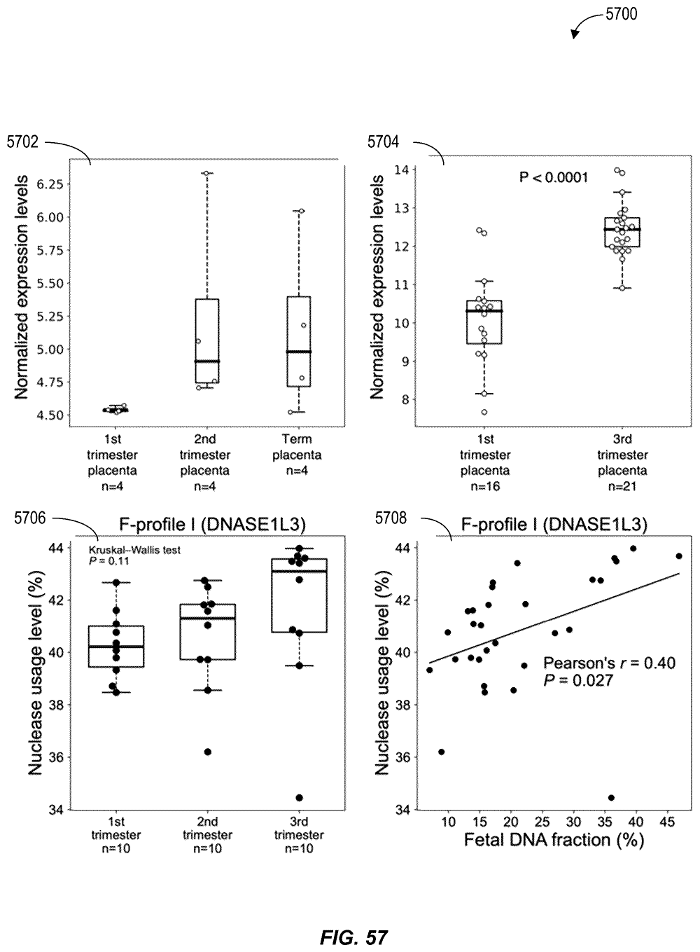

FIG. 57 shows a set of graphs 5700 that identify the nuclease usage levels in plasma cell-free DNA of pregnant women. Boxplots 5702 and 5704 show DNASE1L3 expression levels in the placenta of pregnant women across different trimesters. Boxplot 5706 shows F-profile I (DNASE1L3) contributions in the maternal plasma cell-free DNA of pregnant women across first, second. And third trimesters. Graph 5708 shows correlation between the fetal DNA fractions and F-profile I (DNASE1L3) levels. A shown in the boxplots 5702 and 5704, in placental tissues, an upregulation of DNASE1L3 gene expression levels along gestational age was observed on the basis of transcriptomic data (Mikheev et al. Reprod. Sci. 2008; 15:866-877; Sitras et al. PLOS One 2012; 7:e33294).

The NMF-based nuclease usage level analysis can be used to estimate gestational age based on the certain F-profiles of cell-free DNA. We analyzed the nuclease usage level based on maternal plasma end motifs using a previously published cohort comprising 30 pregnant women (10 in each trimester) (Jiang et al. Clin. Chem. 2017; 63:606-608). As shown in the boxplot 5706, we observed the F-profile I (DNASE1L3) level in maternal plasma cell-free DNA increased progressively over gestational ages across the first trimester (median: 40.2%; range: 38.5-42.7%), second trimester (median: 41.3%; range: 36.2-42.8%) and third trimester (median: 43.1%; range: 34.5-44.0%).

Nuclease usage level analysis disclosed herein could also be feasible for determining the fractional contribution of fetal DNA in plasma samples. As shown in the graph 5708, the F-profile I (DNASE1L3) levels in maternal plasma cell-free DNA were significantly correlated with fetal DNA fractions estimated by an SNP-based approach (Pearson's r=0.40, P=0.027). Hence, the nuclease usage level analysis may be useful for monitoring a physiological status such as pregnancy.

### B. Relationship Between Oxidative Stress and Gestational Age

Apart from cancer patients, we also studied the plasma from pregnant women from the first (n=10), second (n=10), and third trimesters (n=10). The previous study has elucidated that the oxidative stress in the placenta was reported to decline as the gestational age increased (Basu et al. Obstet Gynecol Int 2015; 2015:276095).

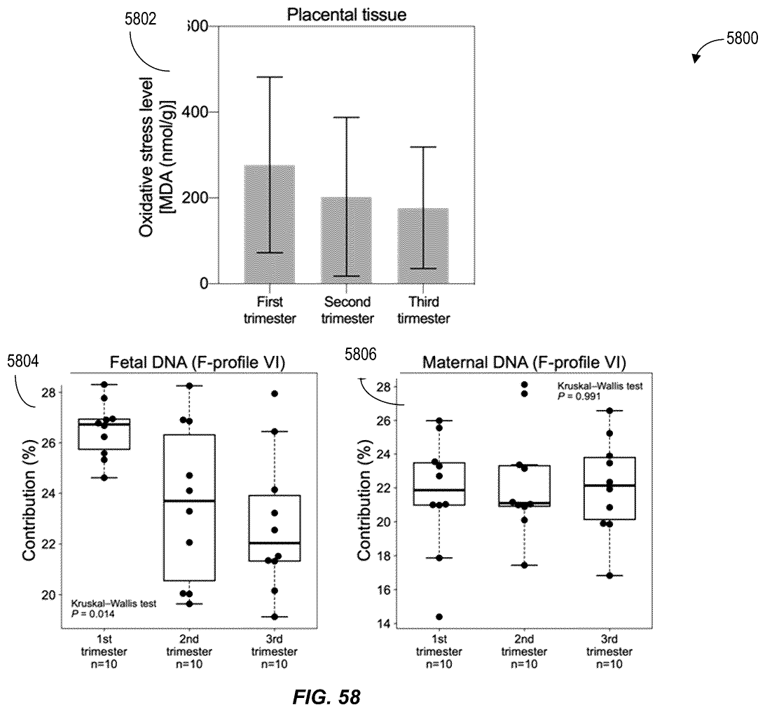

FIG. 58 shows a set of graphs 5800 that identify F-profile analysis and oxidative stress level in pregnant women. Graph 5802 shows oxidative stress levels in placental tissues from pregnant women of different trimesters. Boxplot 5804 shows F-profile VI contributions in fetal-specific DNA in the plasma of pregnant women across different trimesters. Boxplot 5806 shows F-profile VI contributions in maternal-specific DNA in the plasma of pregnant women across different trimesters. As shown in the graphs of FIG. 58, as the trimester increased, the median of F-profile VI level in fetal-specific DNA significantly decreased (First: 26.7%; second: 23.7%; third: 22.0%) (P=0.014, Kruskal-Wallis test), while no significant changes were found in F-profile VI level in maternal-specific DNA. These data indicated that F-profile VI level could indicate the contribution of cell-free DNA originating from oxidative stress induced fragmentation.

### C. Methods for Estimating Gestational Age Based on F-Profiles of Cell-Free DNA

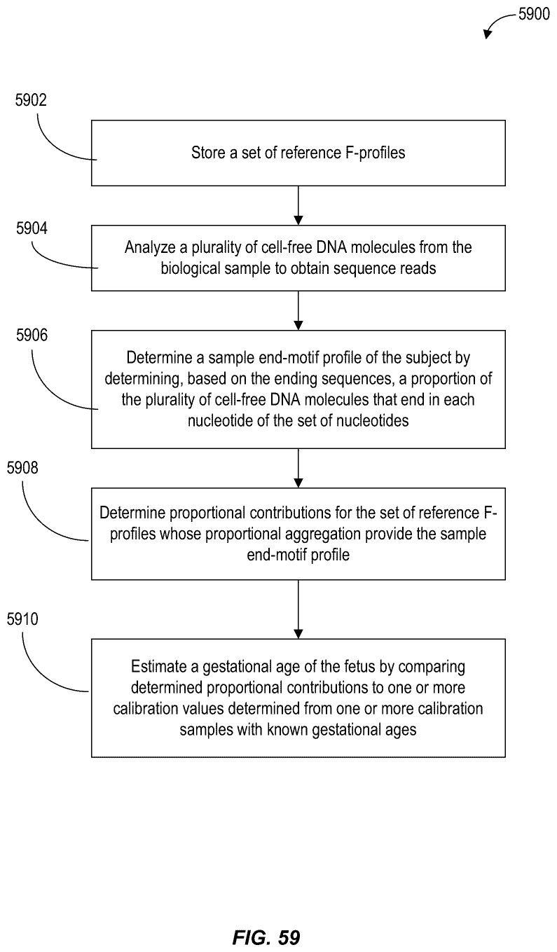

FIG. 59 shows a flowchart of a method 5900 for estimating gestational age based on F-profiles of cell-free DNA molecules, according to some embodiments. The biological sample is obtained from a female subject pregnant with a fetus. The biological sample may be a sample with cell-free DNA molecules from the female subject and the fetus, such as, a plasma, serum, urine, saliva, cerebrospinal fluid, pleural fluid, amniotic fluid, peritoneal fluid, or ascitic fluid sample. Aspects of method 5900 and any other methods described herein may be performed by a computer system. Aspects of method 5900 may be performed in a similar manner as method 5400 of FIG. 54.

At block 5902, a set of reference F-profiles are stored. Each reference F-profile of the set identifies, for each nucleotide of a set of nucleotide, a proportion of cell-free DNA molecules that end in the nucleotide. In addition, each reference F-profile is associated with a type of fragmentation factors. The type of fragmentation factor identifies a particular enzyme (e.g., DNASE1L3, DNASE1), protein (e.g., DFFB), or other biological components or processes that cause fragmentation in cell-free DNA molecules. In some instances, the set of reference F-profiles include 1, 2, 3, 4, 5, 6, 7, 8, 9, 10, 15, 20, 25, 30, 40, 45, 50, or more than 50 F-profiles. For example, the set of reference F-profiles can include six F-profiles I-VI. Block 5902 may be performed in a similar manner as block 5402 of FIG. 54.

At block 5904, a plurality of cell-free DNA molecules from the biological sample are analyzed to obtain sequence reads. The sequence reads include ending sequences corresponding to ends of the plurality of cell-free DNA molecules. The sequence reads can include ending sequences corresponding to ends of the plurality of cell-free DNA fragments. Block 5904 may be performed in a similar manner as block 5404 of FIG. 54.

At block 5906, a sample end-motif profile of the subject is determined by determining, based on the ending sequences, a proportion of the plurality of cell-free DNA molecules that end in each nucleotide of the set of nucleotides. The sample end-motif profile identifies relative frequencies of a plurality of end motifs corresponding to the ending sequences of the plurality of cell-free DNA molecules. The plurality of end motifs can correspond to all feasible combinations of N base positions. For example, if the plurality of end motifs correspond to 4-mers, the plurality of end motifs of the sample end-motif profile can include 256 4-mer combinations (e.g., CCCA, TTCC). Block 5906 may be performed in a similar manner as block 5406 of FIG. 54.

At block 5908, proportional contributions for the set of reference F-profiles is determined whose proportional aggregation provide the sample end-motif profile. The proportional contributions of the set of reference F-profiles sum to one. Block 5908 may be performed in a similar manner as block 5408 of FIG. 54.

The set of reference F-profiles include a first reference F-profile that correlates with the gestational age, e.g., as determined using calibration samples whose gestational age is known. FIGS. 57 and 58 provides such an examples, where the first reference F-profile is F-profile I (which corresponds to DNASE1L3) or F-profile IV. A first proportional contribution for the set of reference F-profiles can correspond to the first reference F-profile.

At block 5910, a gestational age of the fetus is estimated by comparing the first proportional contribution corresponding to the first reference F-profile to one or more calibration values determined from one or more calibration samples with known gestational ages. Aspects of block 5910 can be performed in a similar manner as block 2106 and block 5610. The first reference F-profile can correspond to a particular type of nuclease, e.g., DNASE1L3, as shown in FIG. 57. A reference F-profile can also be used that does not correspond to a nuclease, e.g., F-profile VI, as shown in FIG. 58.

As an example, the proportional contribution(s) of a reference F-profile (e.g., F-profile I representing DNASE1L3) of the calibration data point(s) may be plotted on a chart and form clusters for different gestational ages, and the determined proportional contributions of the biological sample may also be plotted on the chart to determine the cluster that the biological sample falls in. Any of the one or more reference F-profiles can be the first reference F-profile, as long as it correlates to the gestational age. Accordingly, known proportional contribution(s) of one or more reference F-profiles in the calibration sample(s) can be used as calibration data points for determining the gestational age.

Accordingly, some embodiments can, for each calibration sample of the one or more calibration samples, measure the gestational age in the calibration sample and measure a proportional contribution of the first reference end-motif profile determined for the calibration sample. As examples, menstrual history and ultrasonography are two ways to measure gestational age. For example, gestational age can be estimated based on the date of the last menstrual period. Conception can be assumed to occur on day 14 of the cycle, which can be influenced by the variation of ovulation between the menstrual cycles and between individuals. Ultrasound measurement of the embryo or fetus in the first trimester can be the most accurate method to establish gestational age. Gestational age may be estimated from ultrasound using various parameters such as mean sac diameter (MSD), crown-rump length (CRL), biparietal diameter (BPD), and head circumference (HC).

The proportional contribution of the first reference end-motif profile can be used as a calibration value. Proportional contributions of all of the set of references F-profiles can be determined, thereby determining multiple calibration values, e.g., when multiple reference F-profiles are used to estimate the gestational age. The skilled person will appreciate that the gestational age can be measured in various ways.

In some instances, the proportional contribution(s) determined for the one or more of the reference F-profiles (including the first proportional contribution for the first reference F-profile) can be compared to a calibration curve (composed of the calibration data points), and thus the comparison can identify the point on the curve having the known proportional contributions for the biological sample. The gestational age corresponding to the identified point can then be used to estimate the gestational age. For example, the determined proportional contributions can be provided as an input to the calibration function (e.g., a linear or non-linear fit) to obtain an output of the gestational age.

In some embodiments, multiple proportional contributions can be used, as mentioned above. In such an instance, a calibration curve can be a calibration surface in two or more dimensions. Accordingly, estimating the gestational age can include comparing one or more additional proportional contributions to one or more additional calibration values determined from the one or more calibration samples whose gestational ages are known.

### XI. CLASSIFICATION OF PATHOLOGY BASED ON F-PROFILES IN CFDNA

The F-profiles can also be used to classify a level of a pathology in a subject. Examples of a pathology are autoimmune disorders (e.g., SLE) and cancers.

### A. Systemic Lupus Erythematosus (SLE)

The nuclease usage level analysis can be used to differentiate human subjects with and without DNASE1L3 deficiency based on the certain F-profiles of cell-free DNA. Human subjects with DNASE1L3 deficiency would develop Systemic Lupus Erythematosus (SLE)-like symptoms with childhood onset, which was also referred to as the familial SLE (Chan et al. Am. J. Hum. Genet. 2020; 107:882-894). We investigated the nuclease usage level by analyzing plasma cell-free DNA from patients with both copies of DNASE1L3 gene carrying genetic mutations (i.e., DNASE1L3-deficient) (n=10), parents of these patients (n=3) carrying one copy of a mutant DNASE1L3 gene (i.e., the other copy was able to function), and healthy control subjects (n=8) (Chan et al. Am. J. Hum. Genet. 2020; 107:882-894).

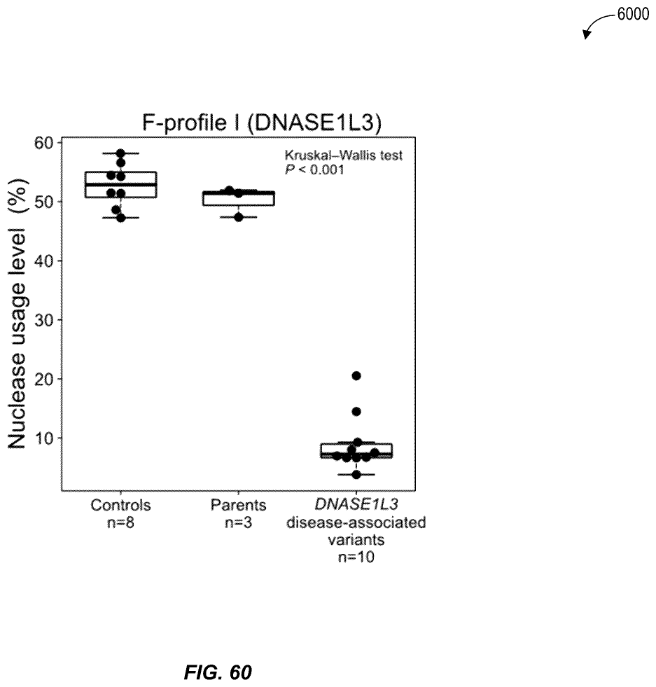

FIG. 60 shows a boxplot 6000 of F-profile I (DNASE1L3) levels for healthy subjects, patients with DNASE1L3 deficiency, and parents of the patients. As shown in FIG. 60, F-profile I (DNASE1L3) in plasma cell-free DNA of patients with DNASE1L3 deficiency appeared to significantly decline (median: 7.3%; range: 3.8-20.5%) compared with their parents (median: 51.4%; range: 47.4-51.9%) and healthy subjects (median: 52.9%; range: 47.3-58.2%) (P<0.0001, Kruskal-Wallis test).

The nuclease usage level analysis could differentiate human subjects with and without SLE.

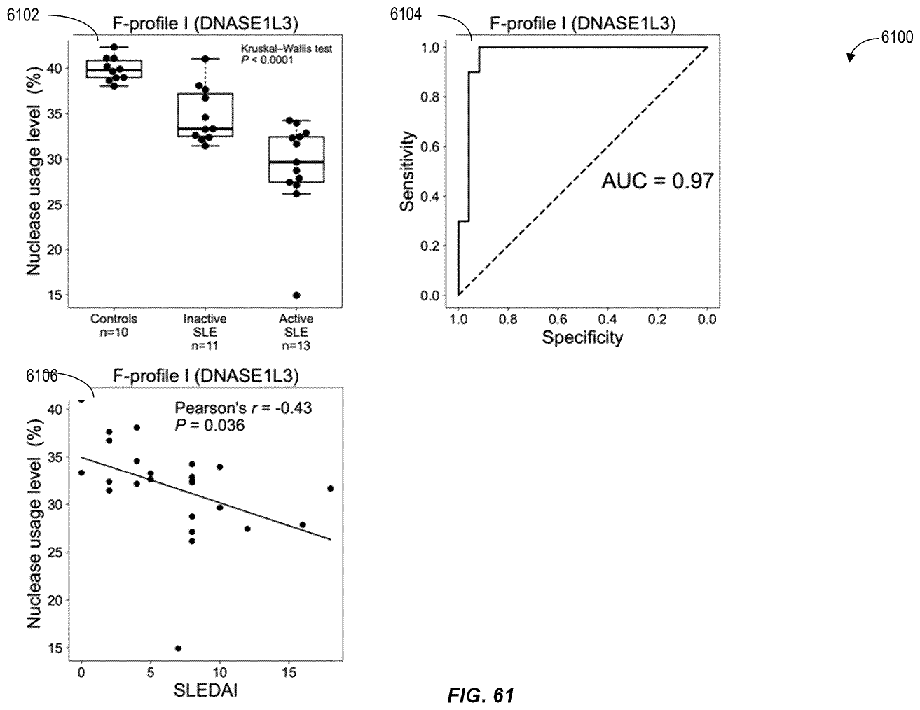

FIG. 61 show a set of graphs 6100 that identify nuclease usage levels in plasma cell-free DNA of subjects with and without systemic lupus erythematosus (SLE). Boxplot 6102 shows F-profile I levels (DNASE1L3) in plasma cell-free DNA across healthy control subjects, patients with inactive SLE and patients with active SLE. ROC curve 6104 shows an assessment of differentiation between patients with and without SLE using F-profile I (DNASE1L3).

A graph 6106 shows a correlation between the Systemic Lupus Erythematosus Disease Activity Index (SLEDAI) and F-profile I levels (DNASE1L3) in patients with SLE. In a cohort comprising 10 healthy controls, 13 and 11 patients with active and inactive sporadic SLE (Chan et al. Proc Natl Acad Sci USA. 2014; 111:E5302-E5311), the boxplot 6102 shows that the DNASE1L3 usage levels gradually decreased across healthy subjects (median: 39.8%; range: 38.0-42.3), patients with inactive SLE (median: 33.3%; range: 31.4-41.0%), and patients with active SLE (median: 29.7%; range: 14.9-34.2%) (P<0.0001, Kruskal-Wallis test). As shown in the ROC curve 6104, the metric of DNASE1L3 usage level (F-profile I) enabled the differentiation between human individuals with and without SLE, with an AUC of 0.97.

In addition, as shown in the graph 6106, the DNASE1L3 usage levels showed a negative correlation with the Systemic Lupus Erythematosus Disease Activity Index (SLEDAI) (Pearson's r: −0.43; P=0.036). Hence, the metric of DNASE1L3 usage level (F-profile I) would inform the presence of autoimmune diseases, as well as facilitate the monitoring of the disease progression.

### B. Cancer

In addition to SLE, the nuclease usage level analysis disclosure can be used to differentiate human subjects with and without hepatocellular carcinoma (HCC). Patients with HCC were reported to be affected by DNASE1L3 activities (Jiang et al. Cancer Discov. 2020; 10:664-73). In connection with the relationship between DNASE1L3 and HCC, the nuclease usage level analysis was applied to a cohort consisting of 38 healthy controls, 17 HBV carriers without HCC, and 34 patients with HCC from a previous study (Jiang et al. Cancer Discov. 2020; 10:664-73).

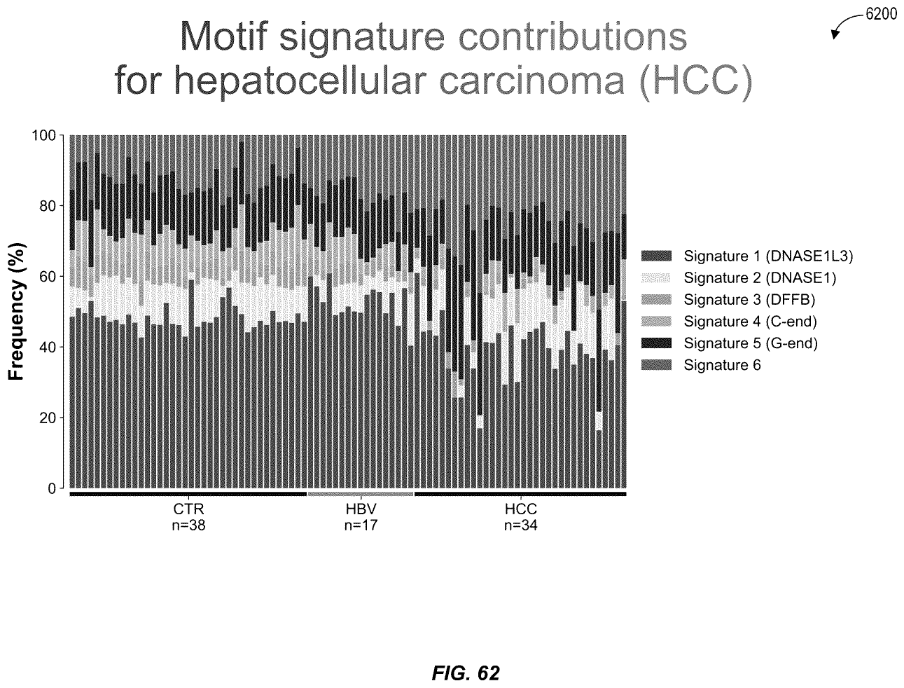

FIG. 62 shows proportional contributions 6400 of F-profiles across normal, HBV, and HCC plasma samples, according to some embodiments. The deconvolution process in FIG. 50 was applied to the normal, HBV and HCC plasma samples from human subjects. As shown in FIG. 62, the samples from control subjects included a relatively high contribution of F-profile I. Similarly, the samples obtained from HBV patients also included a relatively high contribution of F-profile I. By contrast, the HCC samples included a relatively lower contribution from F-profile I. The data shown in FIG. 62 suggests that diagnosis of HCC can be predicted by analyzing proportional contributions from F-profile I.

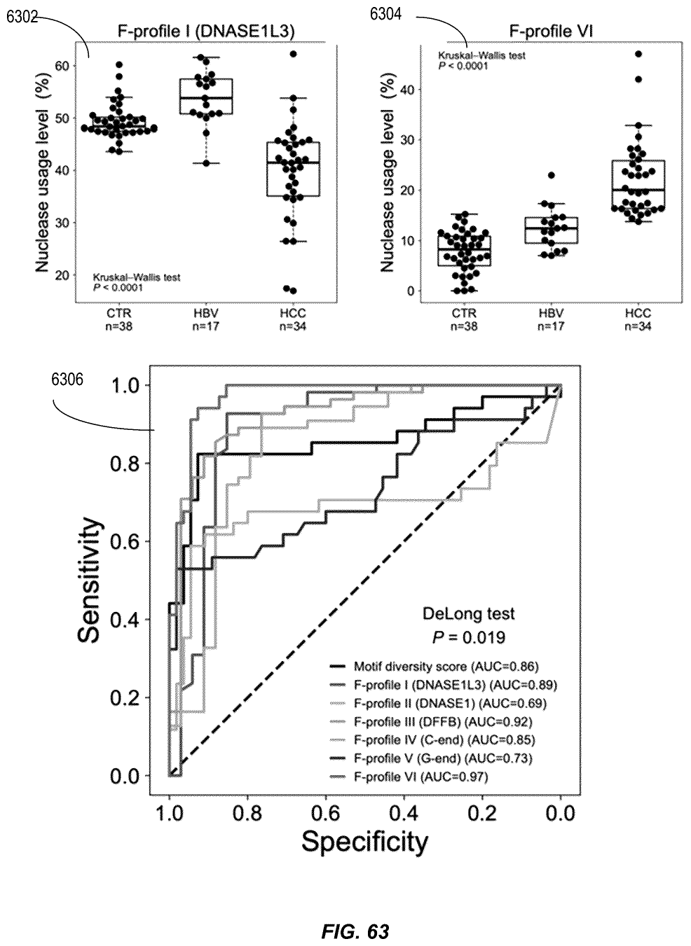

FIG. 63 shows a set of graphs that show nuclease usage levels in plasma cell-free DNA 20 of subjects with and without HCC. Boxplots of F-profile I 6302 and VI 6304 show nuclease levels in plasma cell-free DNA of patients with and without HCC. ROC curves 6306 show an assessment of differentiation between non-HCC and HCC groups using different metrics, including motif diversity score and six F-profiles. Compared with healthy controls, the boxplot 6302 shows that F-profile I (DNASE1L3) usage level was indeed found to be decreased by a median of 6.9% in HCC patients, whereas no appreciable change was observed in HBV carriers.

As shown in the boxplot 6304, we also found a gradual increase of the F-profile VI usage level in HBV carriers and HCC patients. In addition, the ROC curves 6306 shows that, among 6 F-profiles, the most discriminative power in detecting patients with HCC was F-profile VI (AUC: 0.97) which appeared to be random distributions across 256 end motifs (i.e., no obvious preference in end motifs). The performance was superior to the previously reported motif diversity score (AUC: 0.86) (P=0.019, DeLong test), which was used for quantifying the evenness of overall end-motif frequencies (Jiang et al. Cancer Discov. 2020; 10:664-73). These data suggested that the nuclease usage level analysis by simultaneously considering the involvement of multiple nucleases possibly improved the signal-to-noise ratio in detecting diseases.

### C. Relationship Between Disease and Oxidative Stress

As F-profile VI showed a promising differentiation power between patients with and without HCC, there was a consideration of whether any biological implication was linked to the F-profile VI. Because of the nature of F-profile showing the lack of apparent preference in the frequencies of 256 4-mer motifs, one of the possible speculations would be that cell-free DNA fragmentation occurring in patients with cancer might preferentially involve the DNA breaks distinct from the DNA fragmentation induced by the classic apoptotic pathway.

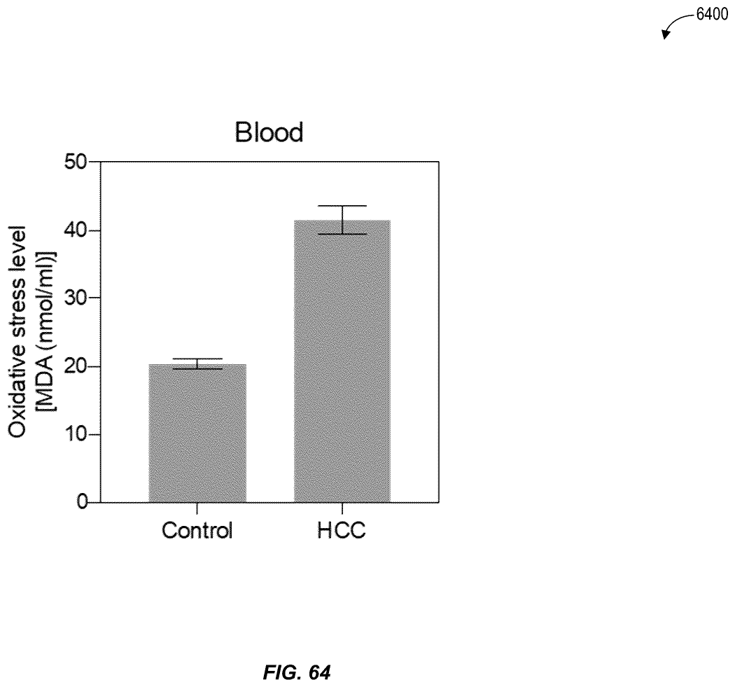

FIG. 64 shows a bar graph 6040 that identifies oxidative stress levels in the blood samples from controls and HCC patients. As shown in FIG. 64, the oxidative stress levels in blood samples with HCC were reported to be higher than that in normal controls (Arsian et al. J. Cancer Ther. 2014; 5:192-197). Based on the above, it can be considered that the F-profile VI is associated with the extent of oxidative stress such that the F-profile VI contribution significantly rose in patients with HCC (see the boxplot 6304 of FIG. 63).

To validate the above hypothesis, we utilized those clinical models in which certain tissues were reported to have higher/lower oxidative stress levels. We first analyzed the plasma cell-free DNA from 15 controls, 25 colorectal cancer (CRC) patients without liver metastasis, and 24 CRC patients with liver metastasis.

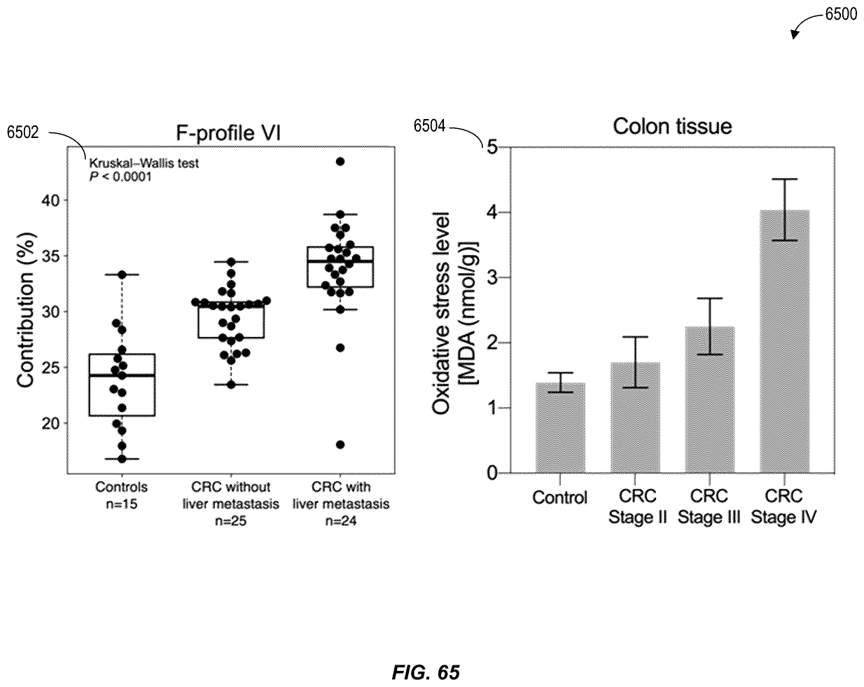

FIG. 65 shows a set of graphs 6500 that provide F-profile analysis and oxidative stress level in CRC patients. Boxplot 6502 shows F-profile VI contributions in controls, CRC patients with and without liver metastasis. Bar graph 6504 shows oxidative stress level in colon tissues from controls and CRC patients of different stages (II-IV). As shown in the boxplot 6502, we indeed found a significant increasing trend of the F-profile VI level from controls (median: 24.3%; range 16.8-33.3%), CRC patients without liver metastasis (median: 30.5%; range 23.4-34.5%), to CRC patients with liver metastasis (median: 34.5%; range 18.1-43.5%) (Kruskal-Wallis test, P<0.0001). Such finding coincided with the report that oxidative stress was increased in patients with colorectal cancer and further enhanced in the patients with advanced stages, as shown in the bar graph 6504 (Skrzydlewska et al. World J Gastroenterol. 2005; 11:403-406;).

### D. Post-Treatment of Disease

Besides CRC patients, we also analyzed the F-profiles in the plasma DNA from 6 patients with Nasopharyngeal carcinoma (NPC) before and after chemoradiotherapy with Cisplatin.

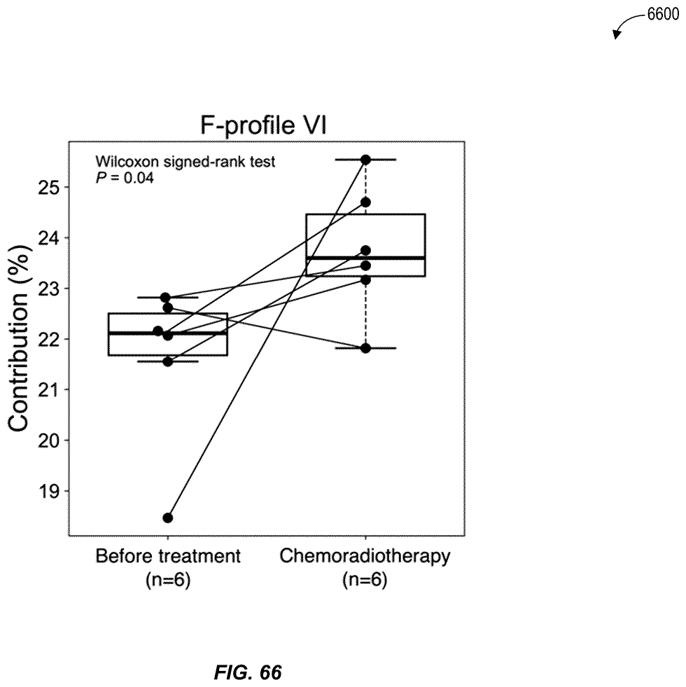

FIG. 66 shows a boxplot 6600 that identifies F-profile VI contributions in NPC patients before and during Chemoradiotherapy treatment with Cisplatin. The increase of oxidative stress was reported to be further enhanced during chemoradiotherapy (Conklin et al. Integr. Cancer Ther. 2004; 3:294-300). As shown in FIG. 66, the F-profile VI level in NPC patients increased during chemoradiotherapy treatment with Cisplatin (median: 22.1%; range: 18.5-22.8%) when compared to the paired patients before treatment (median: 23.6%; range: 21.8-25.5%) (P=0.04, Kruskal-Wallis test).

E. Classifying a Level of Pathology Using F-Profiles cfDNA

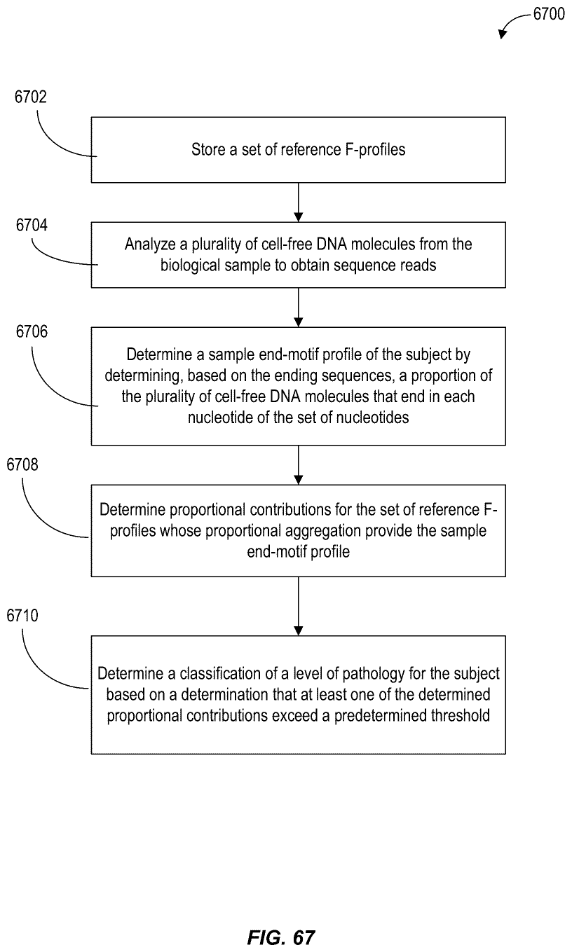

FIG. 67 shows a flowchart of a method 6700 for determining a classification of a level of pathology based on F-profiles of cell-free DNA, according to some embodiments. Example biological samples can be cell-free samples that include cell-free DNA, e.g., blood, plasma, serum, urine, and saliva. The pathology can include cancer (e.g., hepatocellular carcinoma, lung cancer, breast cancer, gastric cancer, glioblastoma multiforme, pancreatic cancer, colorectal cancer, nasopharyngeal carcinoma, head and neck squamous cell carcinoma, etc.) and an auto-immune disorder (e.g., systemic lupus erythematosus). Aspects of method 6700 may be performed in a similar manner as method 5400 of FIG. 54.

At block 6702, a set of reference F-profiles are stored. Each reference F-profile of the set identifies, for each nucleotide of a set of nucleotide, a proportion of cell-free DNA molecules that end in the nucleotide. In addition, each reference F-profile is associated with a type of fragmentation factors. The type of fragmentation factor identifies a particular enzyme (e.g., DNASE1L3, DNASE1), protein (e.g., DFFB), or other biological components or processes that cause fragmentation in cell-free DNA molecules. In some instances, the set of reference F-profiles include 1, 2, 3, 4, 5, 6, 7, 8, 9, 10, 15, 20, 25, 30, 40, 45, 50, or more than 50 F-profiles. For example, the set of reference F-profiles can include six F-profiles I-VI. Block 6702 may be performed in a similar manner as block 5402 of FIG. 54.

At block 6704, a plurality of cell-free DNA molecules from the biological sample are analyzed to obtain sequence reads. The sequence reads include ending sequences corresponding to ends of the plurality of cell-free DNA molecules. The sequence reads can include ending sequences corresponding to ends of the plurality of cell-free DNA fragments. Block 6704 may be performed in a similar manner as block 5404 of FIG. 54.

At block 6706, a sample end-motif profile of the subject is determined by determining, based on the ending sequences, a proportion of the plurality of cell-free DNA molecules that end in each nucleotide of the set of nucleotides. The sample end-motif profile identifies relative frequencies of a plurality of end motifs corresponding to the ending sequences of the plurality of cell-free DNA molecules. The plurality of end motifs can correspond to all feasible combinations of N base positions. For example, if the plurality of end motifs correspond to 4-mers, the plurality of end motifs of the sample end-motif profile can include 256 4-mer combinations (e.g., CCCA, TTCC). Block 6706 may be performed in a similar manner as block 5406 of FIG. 54.

At block 6708, proportional contributions for the set of reference F-profiles is determined whose proportional aggregation provide the sample end-motif profile. The proportional contributions of the set of reference F-profiles sum to one. Block 6708 may be performed in a similar manner as block 5408 of FIG. 54.

At block 6710, a classification of a level of pathology can be determined for the subject based on a determination that at least one of the determined proportional contributions exceed a predetermined threshold. The predetermined threshold can correspond to a proportional contribution of a particular reference F-profile (e.g., F-profile I, F-profile IV). For example, the classification of the level of pathology can be determined for the subject based on a determination that one of the determined proportional contributions is less than a predetermined threshold, as shown in the boxplot 6302 of FIG. 63. In another example, the classification of the level of pathology can be determined for the subject based on a determination that one of the determined proportional contributions is greater than a predetermined threshold, as shown in the boxplot 6304 of FIG. 63.

The levels of pathology can include no cancer, early stage, intermediate stage, or advanced stage. The classification can then select one of the levels. Accordingly, the classification can be determined from a plurality of levels of cancer that include a plurality of stages of cancer. As examples, the cancer can be hepatocellular carcinoma, lung cancer, breast cancer, gastric cancer, glioblastoma multiforme, pancreatic cancer, colorectal cancer, nasopharyngeal carcinoma, and head and neck squamous cell carcinoma. As an example, the auto-immune disorder can be systemic lupus erythematosus.

In further examples, the level of pathology corresponds to a fractional concentration of clinically-relevant DNA associated with the pathology. For instance, the level of pathology can be cancer and the clinically-relevant DNA can be tumor DNA. The reference value can be a calibration value determined from a calibration sample.

### XII. TREATMENTS

### A. Treatment Selection

Embodiments of the present disclosure can accurately predict disease relapse, thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell transplant, or precision medicine. Based on the determined classification of relapse, a treatment plan can be developed to decrease the risk of harm to the subject and increase overall survival rate. Embodiments may further include treating the subject according to the treatment plan.

### B. Types of Treatments

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### XIII. EXAMPLE SYSTEMS

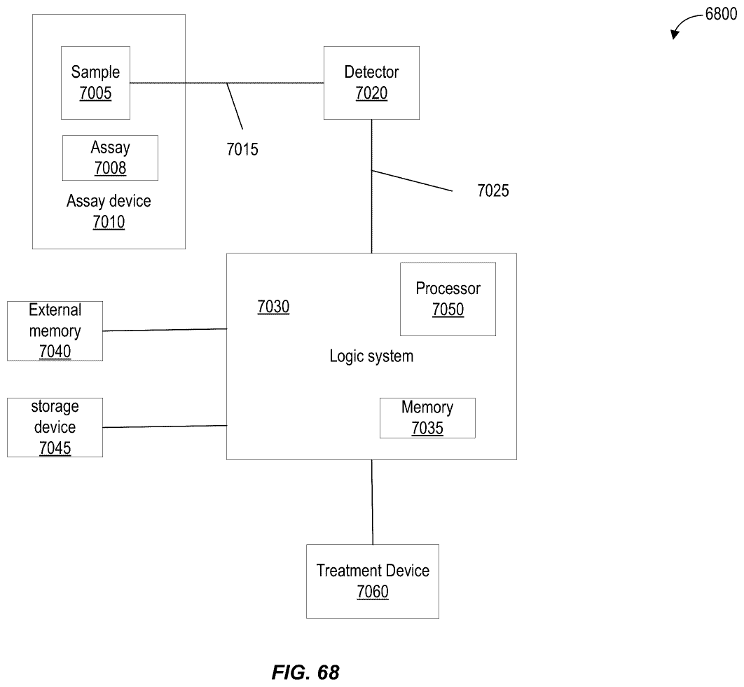

FIG. 68 illustrates a measurement system 6800 according to an embodiment of the present disclosure. The system as shown includes a sample 6805, such as cell-free DNA molecules within an assay device 6810, where an assay 6808 can be performed on sample 6805. For example, sample 6805 can be contacted with reagents of assay 6808 to provide a signal of a physical characteristic 6815. An example of an assay device can be a flow cell that includes probes and/or primers of an assay or a tube through which a droplet moves (with the droplet including the assay). Physical characteristic 6815 (e.g., a fluorescence intensity, a voltage, or a current), from the sample is detected by detector 6820. Detector 6820 can take a measurement at intervals (e.g., periodic intervals) to obtain data points that make up a data signal. In one embodiment, an analog-to-digital converter converts an analog signal from the detector into digital form at a plurality of times. Assay device 6810 and detector 6820 can form an assay system, e.g., a sequencing system that performs sequencing according to embodiments described herein. A data signal 6825 is sent from detector 6820 to logic system 6830. As an example, data signal 6825 can be used to determine sequences and/or locations in a reference genome of DNA molecules. Data signal 6825 can include various measurements made at a same time, e.g., different colors of fluorescent dyes or different electrical signals for different molecule of sample 6805, and thus data signal 6825 can correspond to multiple signals. Data signal 6825 may be stored in a local memory 6835, an external memory 6840, or a storage device 6845.

Logic system 6830 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 6830 and the other components may be part of a stand-alone or network connected computer system, or they may be directly attached to or incorporated in a device (e.g., a sequencing device) that includes detector 6820 and/or assay device 6810. Logic system 6830 may also include software that executes in a processor 6850. Logic system 6830 may include a computer readable medium storing instructions for controlling measurement system 6800 to perform any of the methods described herein. For example, logic system 6830 can provide commands to a system that includes assay device 6810 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

System 6800 may also include a treatment device 6860, which can provide a treatment to the subject. Treatment device 6860 can determine a treatment and/or be used to perform a treatment. Examples of such treatment can include surgery, radiation therapy, chemotherapy, immunotherapy, targeted therapy, hormone therapy, and stem cell transplant. Logic system 6830 may be connected to treatment device 6860, e.g., to provide results of a method described herein. The treatment device may receive inputs from other devices, such as an imaging device and user inputs (e.g., to control the treatment, such as controls over a robotic system).

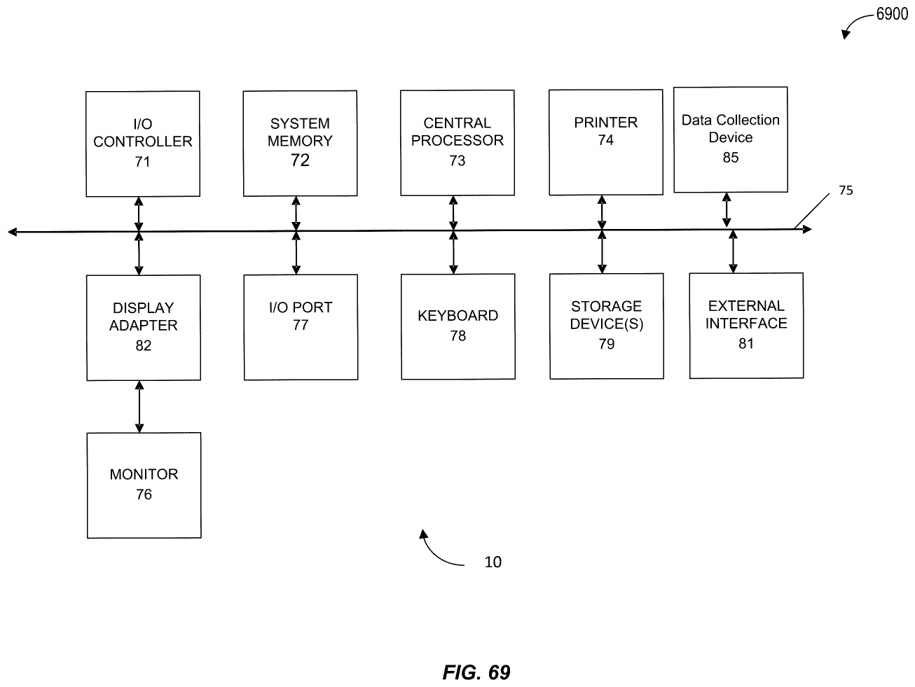

Any of the computer systems mentioned herein may utilize any suitable number of subsystems. Examples of such subsystems are shown in FIG. 69 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 69 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims Efforts have been made to ensure accuracy with respect to numbers used (e.g., amounts, temperature, etc.) but some experimental errors and deviations should be accounted for Unless indicated otherwise, parts are parts by weight, molecular weight is weight average molecular weight, temperature is in degrees Celsius, and pressure is at or near atmospheric.

All patents, patent applications, publications, and descriptions mentioned herein are incorporated by reference in their entirety for all purposes. None is admitted to be prior art. Where a conflict exists between the instant application and a reference provided herein, the instant application shall dominate.

1. A method of estimating a fractional concentration of clinically-relevant DNA molecules in a urine sample of a subject, the urine sample including the clinically-relevant DNA molecules and other DNA molecules that are cell-free, the method comprising:
analyzing a plurality of cell-free DNA molecules from the urine sample, wherein analyzing the plurality of cell-free DNA molecules includes:
determining locations of the plurality of cell-free DNA molecules; and
identifying, based on the locations of the plurality of cell-free DNA molecules, a set of cell-free DNA molecules that are from open chromatin regions of one or more tissues associated with the clinically-relevant DNA molecules;
determining, using the set of cell-free DNA molecules, a relative abundance of the set of cell-free DNA molecules that are from open chromatin regions of the one or more tissues; and
estimating the fractional concentration of the clinically-relevant DNA molecules in the urine sample by comparing the relative abundance to one or more calibration values determined from one or more calibration samples whose fractional concentration of the clinically-relevant DNA molecules are known.

2. The method of claim 1, wherein comparing the relative abundance to the one or more calibration values includes comparing the relative abundance to a calibration curve that includes the one or more calibration values.

3. The method of claim 1, further comprising:
for each calibration sample of the one or more calibration samples:
measuring the fractional concentration of the clinically-relevant DNA molecules in the calibration sample; and
measuring the relative abundance of cell-free DNA molecules from the calibration sample that are from the open chromatin regions of the one or more tissues.

4. The method of claim 3, wherein measuring the fractional concentration of the clinically-relevant DNA molecules uses a tissue-specific allele or a tissue-specific methylation pattern.

5. A method of enriching a urine sample for clinically-relevant DNA molecules, the urine sample including the clinically-relevant DNA molecules and other DNA molecules that are cell-free, the method comprising:
analyzing a plurality of cell-free DNA molecules from the urine sample, wherein analyzing the plurality of cell-free DNA molecules includes:
identifying, from the plurality of cell-free DNA molecules, a set of cell-free DNA molecules that are from open chromatin regions of one or more tissues associated with the clinically-relevant DNA molecules; and
creating an enriched sample using the set of cell-free DNA molecules that are from the open chromatin regions of the one or more tissues, wherein the enriched sample has a higher concentration of clinically-relevant DNA compared to the urine sample.

6. The method of claim 5, further comprising determining a property associated with the clinically-relevant DNA molecules in the enriched sample, wherein the property associated with the clinically-relevant DNA molecules in the urine sample is (1) a fractional concentration of the clinically-relevant DNA molecules or (2) a level of pathology of a subject from whom the urine sample was obtained, the level of pathology associated with the clinically-relevant DNA molecules.

7. The method of claim 5, wherein creating the enriched sample further includes using the set of cell-free DNA molecules that are from the open chromatin regions of the one or more tissues and that have sizes that are less than a specified size threshold.

8. The method of claim 7, wherein the specified size threshold is 40 base pairs, 50 base pairs, 60 base pairs, 70 base pairs, 80 base pairs, 90 base pairs, 100 base pairs, 110 base pairs, 120 base pairs, 130 base pairs, 140 base pairs, 150 base pairs, or 160 base pairs.

9. The method of claim 5, wherein creating the enriched sample further includes using the set of cell-free DNA molecules that are from the open chromatin regions of the one or more tissues and that have one or more ending sequences that correspond to a sequence end signature.

10. The method of claim 5, wherein identifying the set of cell-free DNA molecules or creating the enriched sample includes:
subjecting the plurality of cell-free DNA molecules to probe molecules that have sequences from the open chromatin regions, thereby obtaining the set of cell-free DNA molecules.

11. The method of claim 10, wherein creating the enriched sample includes:
amplifying the set of cell-free DNA molecules using the one or more probe molecules.

12. The method of claim 10, wherein creating the set of cell-free DNA molecules includes:
capturing the set of cell-free DNA molecules using the one or more probe molecules; and
discarding other cell-free DNA molecules of the plurality of cell-free DNA molecules.

13. The method of claim 10, wherein one or more probe molecules are attached to a surface.

14-23. (canceled)

24. The method of claim 1, wherein the clinically-relevant DNA molecules are transrenal DNA molecules.

25. The method of claim 1, wherein the clinically-relevant DNA molecules include fetal DNA or tumor DNA.

26-29. (canceled)

30. The method of claim 1, wherein determining the relative abundance includes:
determining a first relative frequency of the set of cell-free DNA molecules that are from the open chromatin regions of the one or more tissues;
determining a second relative frequency of reference sequences of a reference genome that are from the open chromatin regions of the one or more tissues; and
determining the relative abundance based on the first relative frequency and the second relative frequency.

31. The method of claim 30, wherein determining the second relative frequency includes identifying single-nucleotide variants of the reference genome that are from the open chromatin regions of the one or more tissues.

32. The method of claim 30, wherein the relative abundance is a ratio between the first relative frequency and the second relative frequency.

33. The method of claim 1, wherein the relative abundance is an end density of the plurality of cell-free DNA molecules that end in the open chromatin regions of the one or more tissues.

34. The method of claim 33, wherein the end density comprises a first amount of the set of cell-free DNA molecules that are from the open chromatin regions of the one or more tissues divided by a second amount of the plurality of cell-free DNA molecules from one or more other regions.

35. The method of claim 1, wherein determining the locations of the plurality of cell-free DNA molecules includes aligning sequence reads of the plurality of cell-free DNA molecules to a reference genome.

36. The method of claim 1, wherein the one or more tissues includes at least one of heart, lungs, colon, liver, or white blood cells.

37. The method of claim 1, wherein the set of cell-free DNA molecules end at one or more positions in a window around the open chromatin regions of the one or more tissues.

38. The method of claim 1, wherein the open chromatin regions include Dnase1 hypersensitivity sites.

39. The method of claim 1, wherein the set of cell-free DNA molecules include at least 5,000 cell-free DNA molecules.

40-49. (canceled)

50. The method of claim 1, wherein the urine sample is processed using a DNA stabilization agent prior to obtaining the plurality of cell-free DNA molecules from the urine sample.

51. The method of claim 1, wherein analyzing the plurality of cell-free DNA molecules includes receiving sequence reads obtained from a sequencing of the plurality of cell-free DNA molecules.

52-95. (canceled)
